(19) United States
(12) Patent Application Publication
(10) Pub. No.: US 2014/0087463 A1
(43) Pub. Date: Mar. 27, 2014

(54) OPTICAL CONTROL OF CELL SIGNALING

(71) Applicant: The Washington University, St. Louis, MO (US)

(72) Inventors: Narasimhan Gautam, St. Louis, MO (US); Welivitiya Kankanamlage Karunarathne, St. Louis, MO (US); Vani Kalyanaraman, St. Louis, MO (US)

(21) Appl. No.: 14/036,633

(22) Filed: Sep. 25, 2013

Related U.S. Application Data
(60) Provisional application No. 61/705,342, filed on Sep. 25, 2012.

(51) Int. Cl. C12N 5/071

(52) U.S. Cl. CPC C12N 5/0602
USPC 435/375

(57) ABSTRACT

The present invention discloses methods of spatiotemporally controlling G protein signaling in a cell using an artificial optical input. Also disclosed are methods of manipulating cell behavior that is controlled by asymmetrical G protein signaling in a cell.

### CROSS REFERENCE TO RELATED APPLICATIONS

This application claims the priority of U.S. provisional application No. 61/705,342 filed Sep. 25, 2012, which is hereby incorporated by reference in its entirety.

### GOVERNMENTAL RIGHTS

This invention was made with government support under GM069027, GM080558, and GM080558-0251 awarded by the National Institutes of Health. The government has certain rights in the invention.

### FIELD OF THE INVENTION

This invention encompasses methods of spatiotemporally controlling G protein signaling in a cell using an artificial optical input. Also disclosed are methods of manipulating cell behavior that is controlled by asymmetrical G protein signaling in a cell.

### BACKGROUND OF THE INVENTION

Important cell behaviors such as cell migration and early neuron differentiation involve polarization that suggests asymmetric extracellular stimulation of a cell. Therefore, receptor activation in a selected region of a single cell for defined durations of time may allow a complete native signaling network as well as cell behavior to be externally governed. The ability to control cell behavior may be used to direct immune cell migration to target regions such as tumors, guide neuron growth and differentiation to create new connections where such connections have been disrupted by injury or disease, control heart rate for defibrillation in the case of cardiac arrest, or as a pacemaker with arrhythmias, activate signaling in the heart cells to prevent cardiac hypertrophy after myocardial infarction, and control secretion of molecules from neuroendocrine cells like pancreatic cells. However, there are no effective methods to exercise precise spatiotemporal control over receptors stimulated by extracellular signals in a single cell. Therefore, there is a need for methods for spatiotemporally controlling cell behavior.

### SUMMARY OF THE INVENTION

One aspect of the present invention encompasses a method of modulating localized G protein signaling in a cell using an artificial optical input. The method comprises (a) introducing at least one exogenous opsin into a cell, wherein (i) the exogenous opsin comprises a light sensing domain of a melanopsin or a metazoan color opsin and a G protein coupled receptor (GPCR) activation domain that effects G protein signaling (ii) and introducing exogenous opsin into a cell comprises introducing an amino acid sequence comprising an opsin into the cell, introducing a nucleic acid sequence capable of expressing an opsin into the cell, or a combination thereof; and (b) changing an artificial optical input in a localized region on the cell's surface. The activation state of the exogenous opsin within the localized region is affected when the light sensing domain detects a change in the artificial optical input, thereby resulting in the GPCR activation domain modulating G protein signaling. Typically, the GPCR activation domain can activate a G protein comprising a Gα subunit selected from the group consisting of a Gαs subunit, a Gαi/o subunit, a Gαq subunit, and Gα12/13.

Another aspect of the present invention encompasses a method of modulating cell behavior that is controlled by localized G protein signaling in the cell. The method comprises (a) introducing at least one exogenous opsin into a cell, wherein (i) the exogenous opsin comprises an light sensing domain of a melanopsin or a metazoan color opsin and a G protein coupled receptor (GPCR) activation domain that effects G protein signaling, and (ii) introducing exogenous opsin into a cell comprises introducing an amino acid sequence comprising an opsin into the cell, introducing a nucleic acid sequence capable of expressing an opsin into the cell, or a combination thereof; and (b) changing an artificial optical input in a localized region on the cell's surface. The activation state of the exogenous opsin within the localized region is affected when the light sensing domain detects a change in the artificial optical input, thereby resulting in the GPCR activation domain modulating G protein signaling and cell behavior. Typically, the GPCR activation domain can activate a G protein comprising a Gα subunit selected from the group consisting of a Gαs subunit, a Gαi/o subunit, a Gαq subunit, and Gα12/13.

Another aspect of the present invention encompasses a method of modulating localized G protein signaling in at least one cell in a tissue using an artificial optical input. The method comprises (a) introducing at least one exogenous opsin into a cell, wherein (i) the exogenous opsin comprises an light sensing domain of a melanopsin or a metazoan color opsin and a G protein coupled receptor (GPCR) activation domain that effects G protein signaling, and (ii) introducing exogenous opsin into a cell comprises introducing an amino acid sequence comprising an opsin into the cell, introducing a nucleic acid sequence capable of expressing an opsin into the cell, or a combination thereof; and (b) changing an artificial optical input in a localized region on the cell's surface. The activation state of the exogenous opsin within the localized region is affected when the light sensing domain detects a change in the artificial optical input, thereby resulting in the GPCR activation domain modulating G protein signaling in at least one cell in the tissue. Typically, the GPCR activation domain can activate a G protein comprising a Gα subunit selected from the group consisting of a Gαs subunit, a Gαi/o subunit, a Gαq subunit, and Gα12/13.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

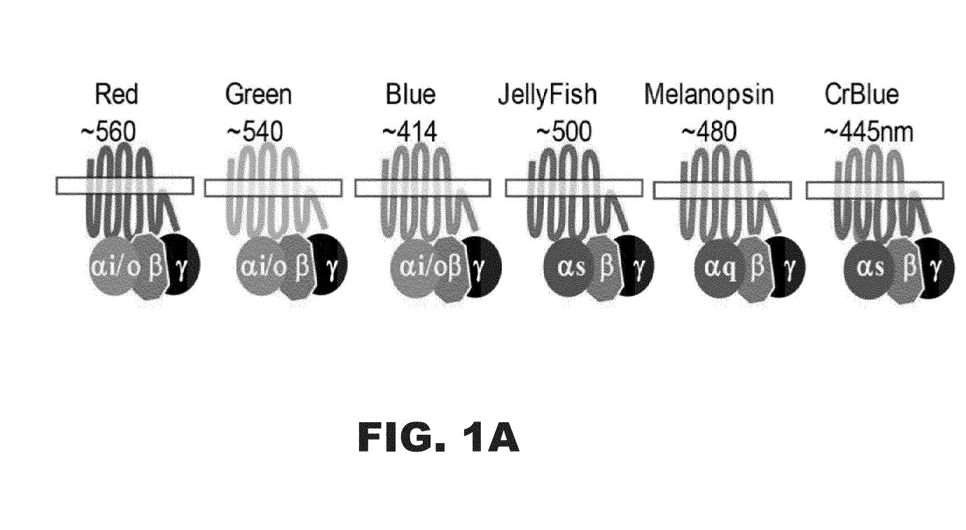

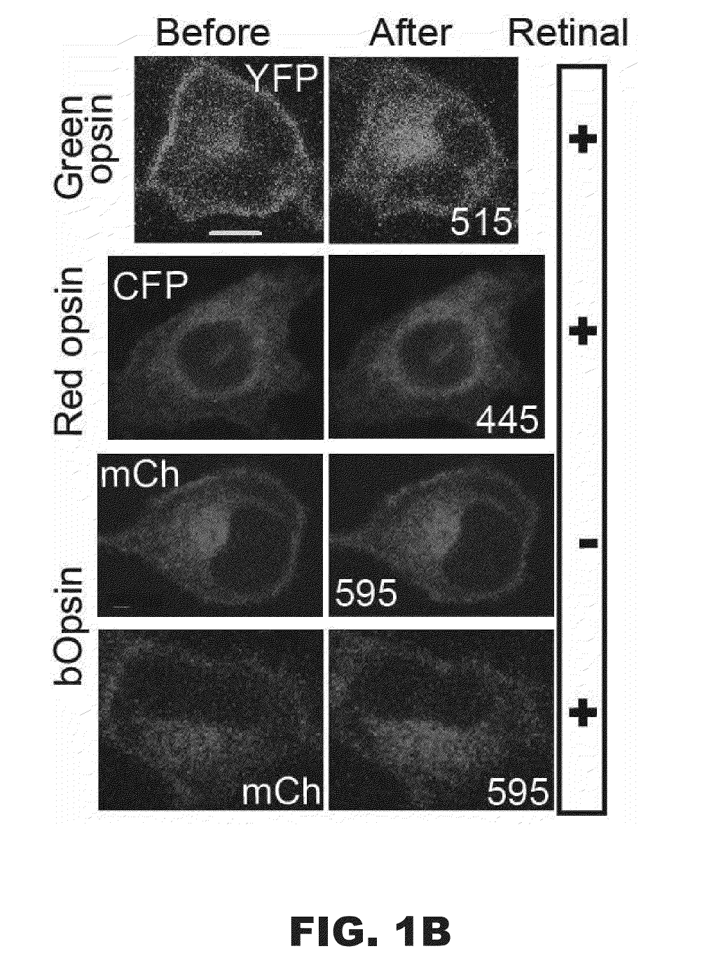

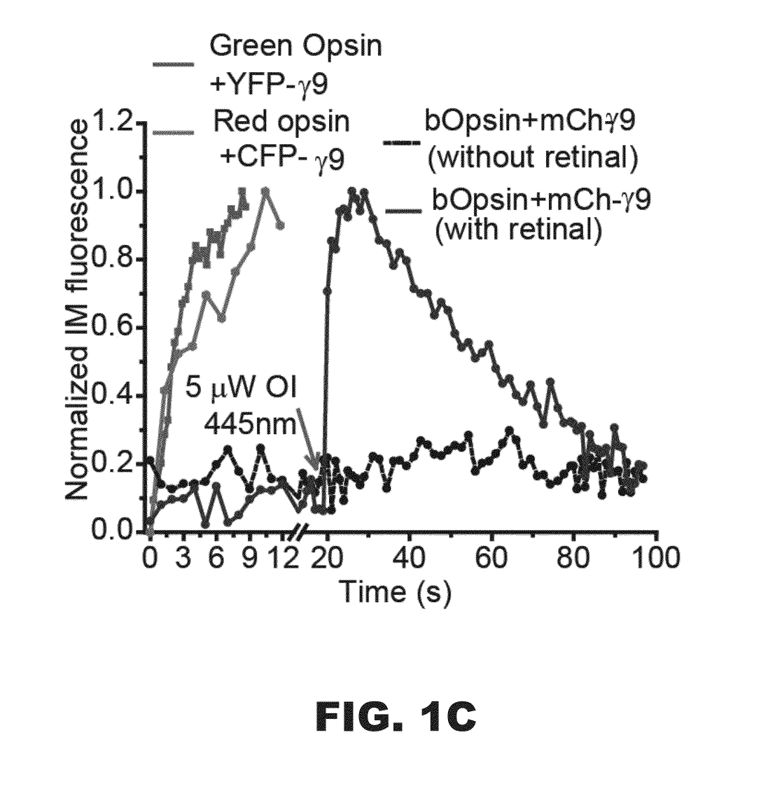

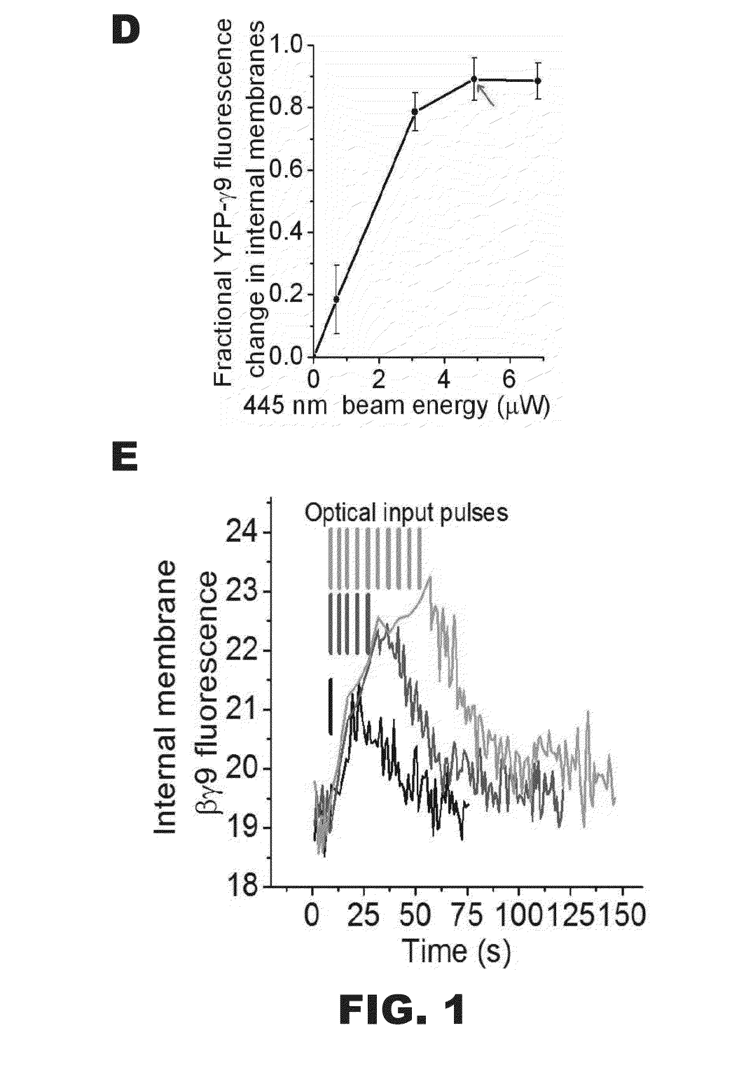

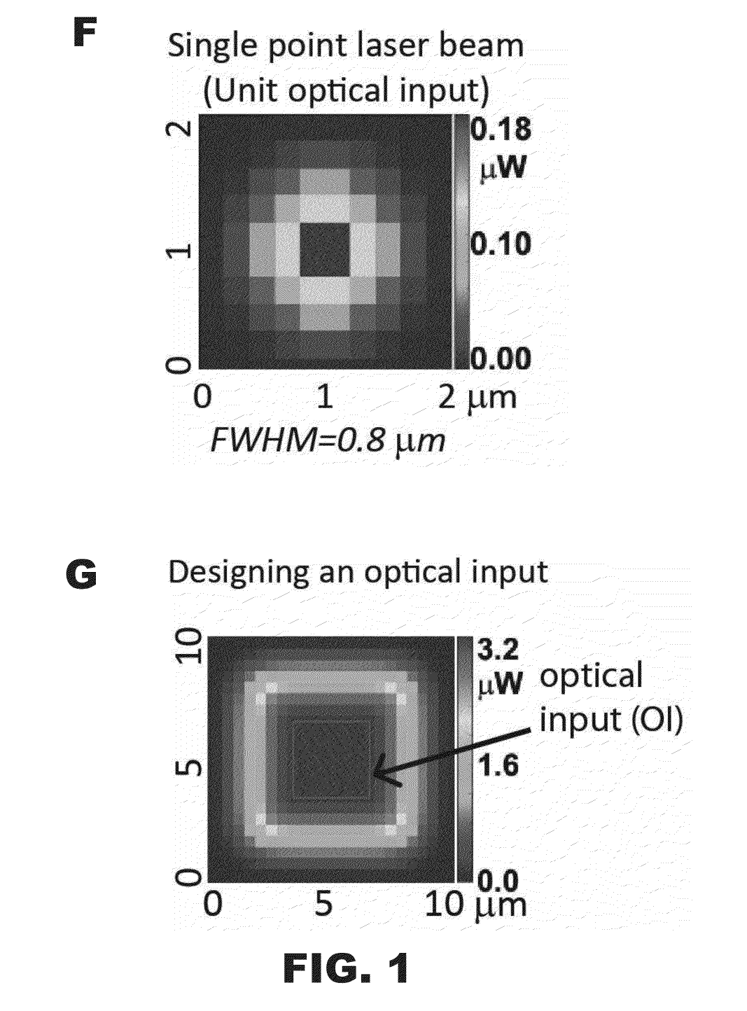

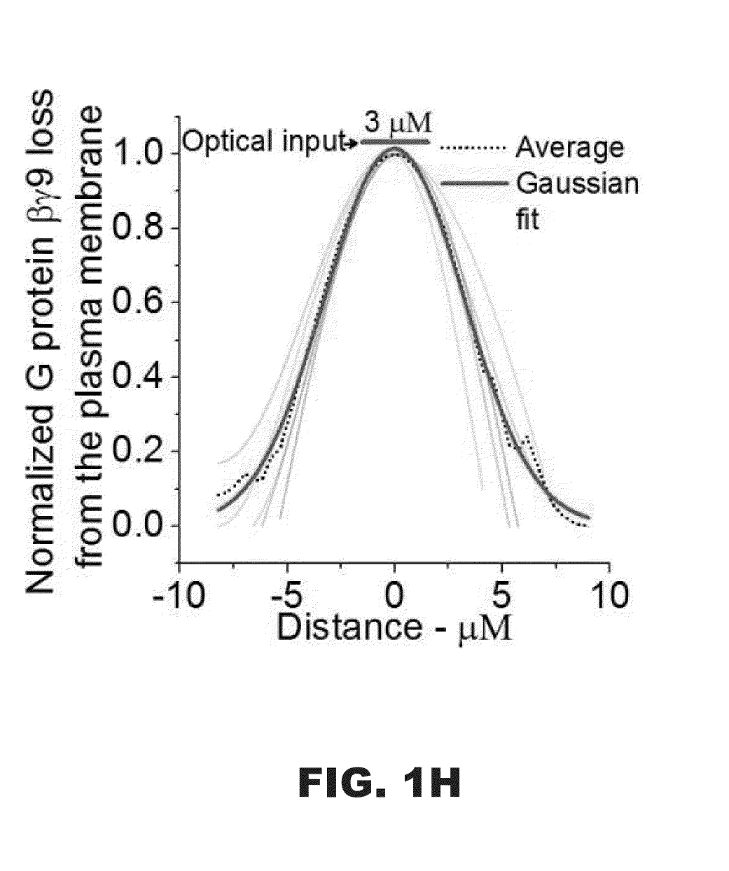

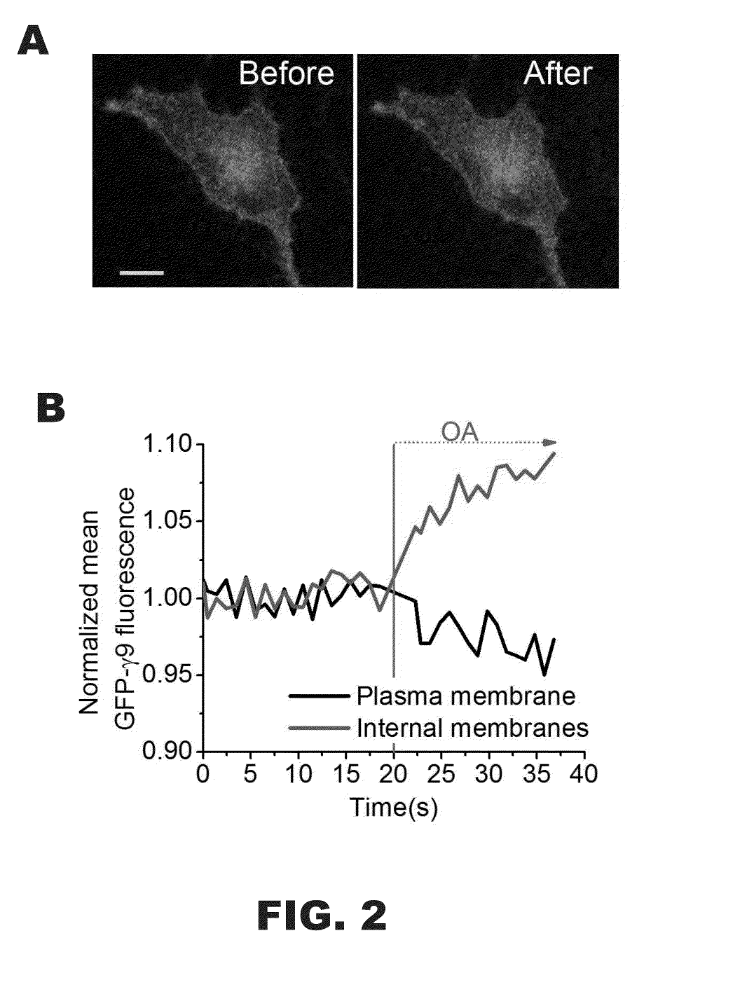

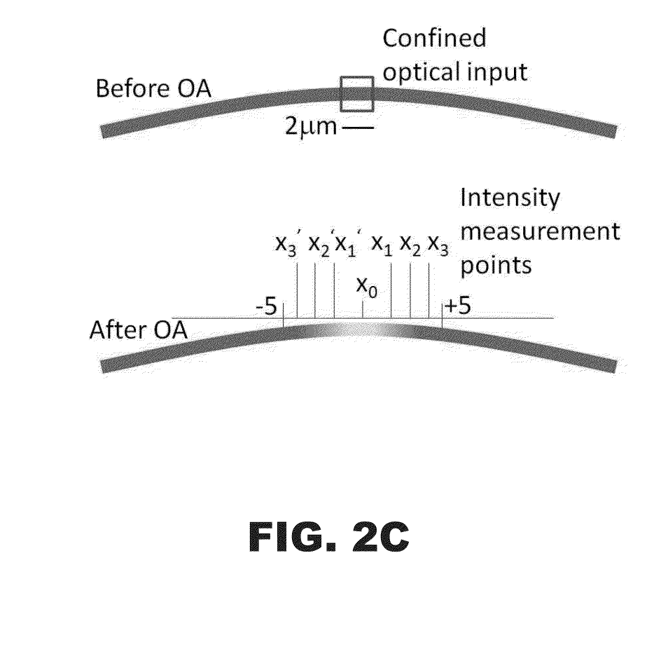

FIG. 1 depicts diagrams plots and images showing optical localization of GPCR activity in a cell with opsins. A, Opsins used, their λmax and Gα subtype specificity. B, Representative images of HeLa cells expressing green opsin and YFP-γ9 (green), red opsin and CFP-γ9 (blue), and bOpsin and mCh-γ9 (red). Cells were incubated with (+) or without (−) 11-cis retinal as indicated. For green opsin and red opsin, FP-γ9 distribution in the first (before) and last (after) images during image capture is shown. For bOpsin, images show mCh-γ9 distribution before and after optical activation (at 20 s after initiating image acquisition) with 445-nm, 5-μW optical inputs. C, Translocation of FP-βγ to intracellular membranes (IM) is plotted (n=8). bOpsin-expressing cells with retinal were activated with a single 5-μW pulse whereas cells without retinal did not show translocation even after optical activation with 30 pulses (n=8). Here and in all optical activation experiments below, n values represent number of cells. Yellow bar here and all images below: 10 μm. D, A single HeLa cell coexpressing bOpsin-mCh and YFP-γ9 was optically activated by varying laser intensities (445 nm). Individual cells were optically activated using a single-pulse OIthat covered the entire cell (energy of the OI in microwatts is indicated on the image). After 20 s the cell was imaged to capture YFP-γ9 distribution. The cell was allowed a 1-min recovery and tested at the next intensity. The plot shows fractional YFP-γ9 intensity changes in internal membranes. The red arrow shows the selected intensity (5 μW) for optical activation of bOpsin in experiments below (n=7). E, Magnitude and duration of γ9 translocation can be controlled by varying the number of pulses in HeLa cells expressing bOpsin and mCh-γ9. The cell was initially imaged for 10 s (baseline reference) and then activated with 1, 5, and 10 (1 pulse every 5 s) OI pulses (5 μW). mCh-γ9 distribution was continually imaged. Plot shows internal membrane fluorescence (please see FIG. 2 also). F and G, Designing an optical input (OI) for opsin activation. (F) Single-point laser beam energy density profile of 445 nm, 5 μW at the image plane. Experimentally a cell can be exposed to this optical input by selecting the crosshair tool (+) as the ROI. (G) Energy density profile of squareshaped OI area (example: 3×3 μm) of laser raster scan. The galvo mirrors scan the ROI at 0.87 ms/μm² and the area of the OI determines the duration of a single pulse. H, Determination of spatial confinement of optically induced GPCR activity using FP-γ9 translocation. Shown is extent of GFP-γ9 translocation from the plasma membrane of HeLa cells expressing bOpsin and GFP-γ9 before and 5 s after application of a confined 3-μmwide OI (purple line). Fractional GFP-γ9 loss was calculated. A fitted Gaussian distribution curve (red line) to the averaged experimental data points (dotted line) resulted in FWHM of 6.3 (n=6).

FIG. 2 depicts images, plots and diagrams showing bOpsin induced γ9 translocation and quantification of confined GPCR activity. A and B, GFP-γ9 in HeLa cells coexpressing bOpsin. Cell wide optical activation of bOpsin (445 nm, 5 μW) induces GFP-γ9 translocation from plasma membrane to intracellular membranes. C, Schematic for the experimental monitoring of spatial confinement of optically induced GPCR activity. Gβγ9 translocation after application of an optical input of 2 μm×2 μm spread to activate the opsin. Extent of Gβγ9 translocation was measured across the activated area (xo) along the plasma membrane before and after OA.

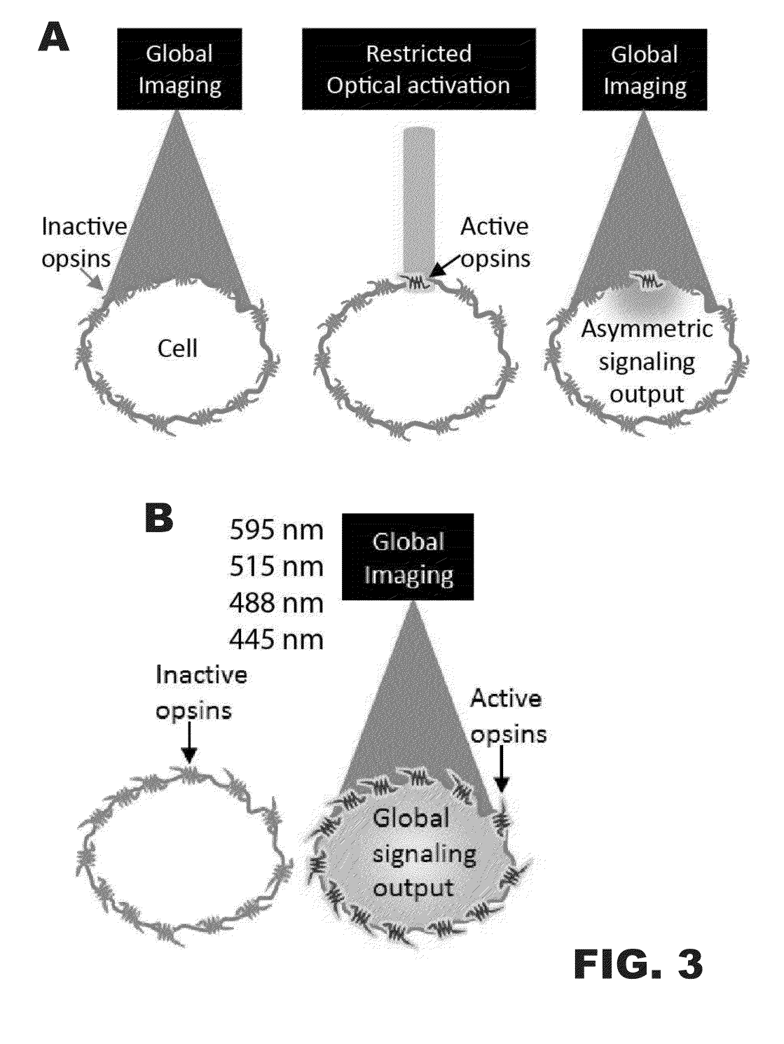

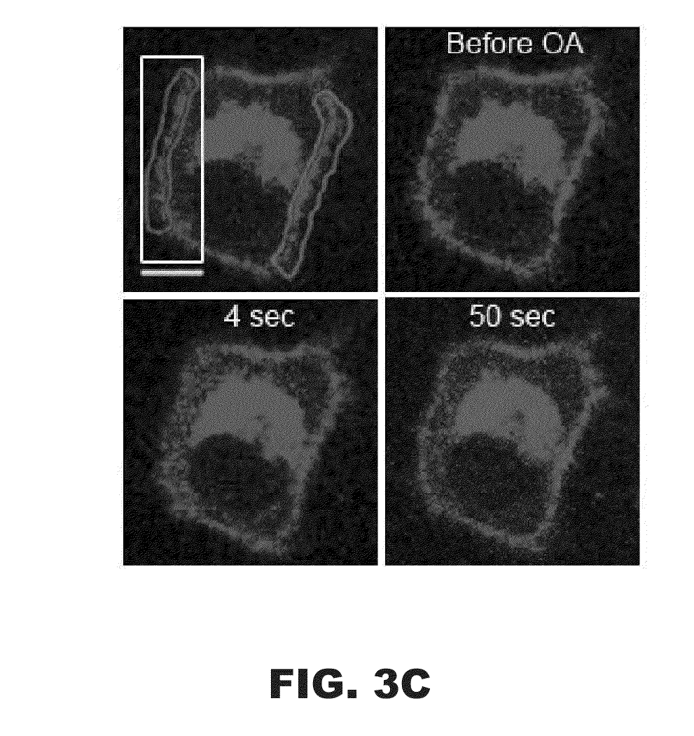

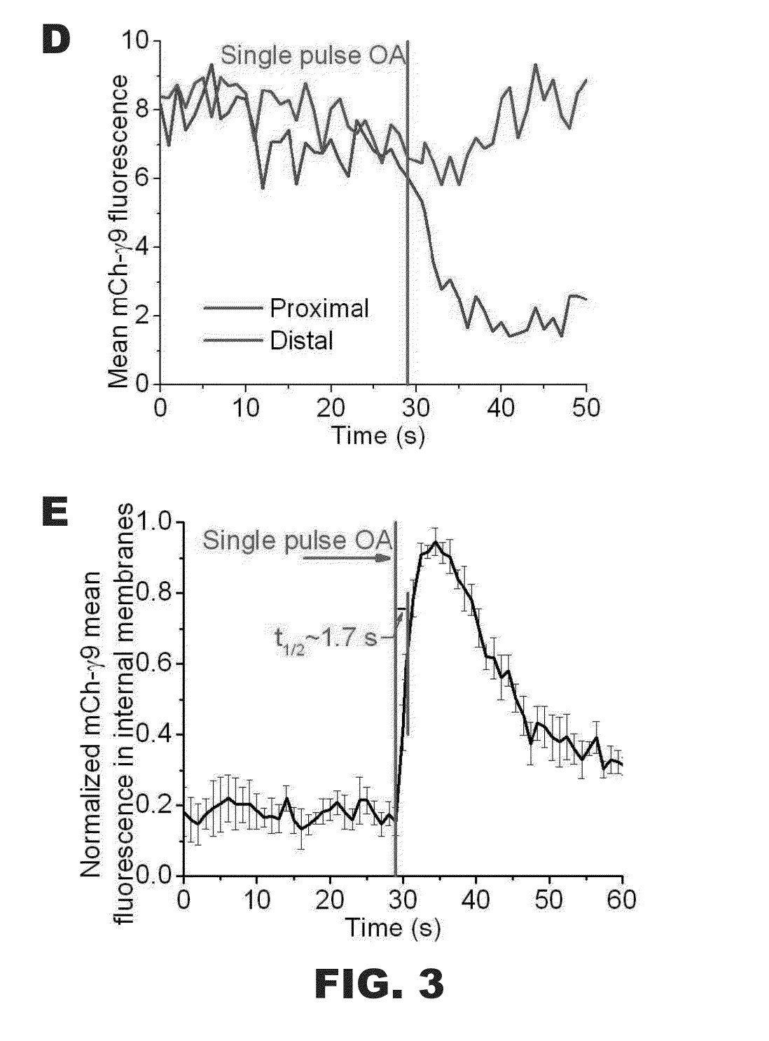

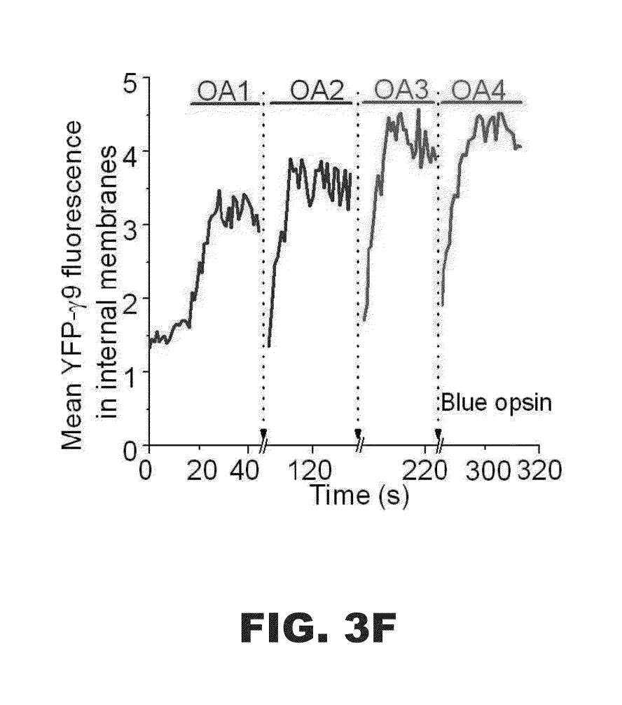

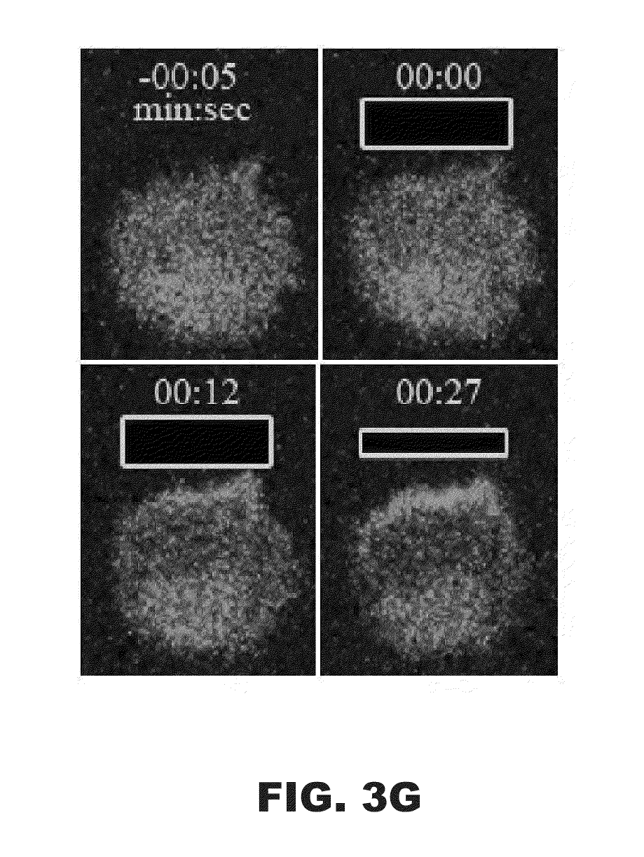

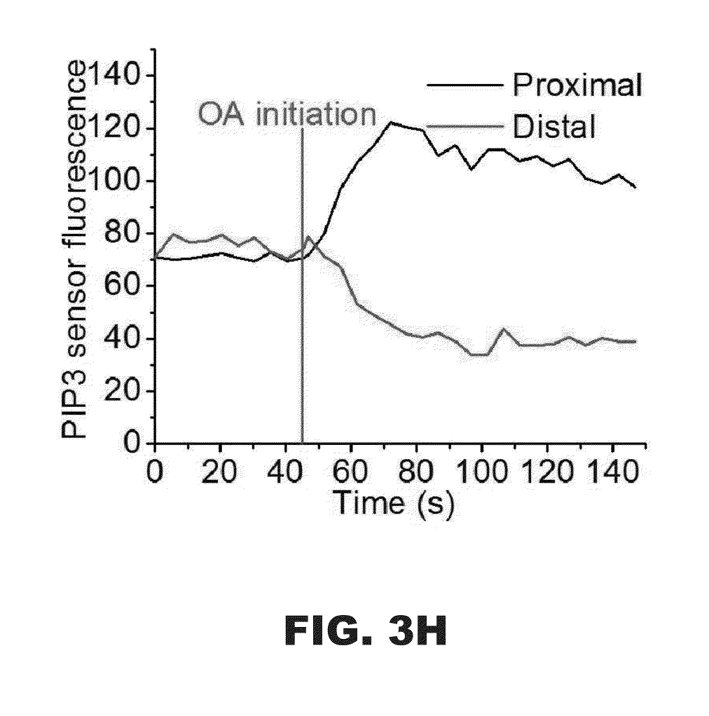

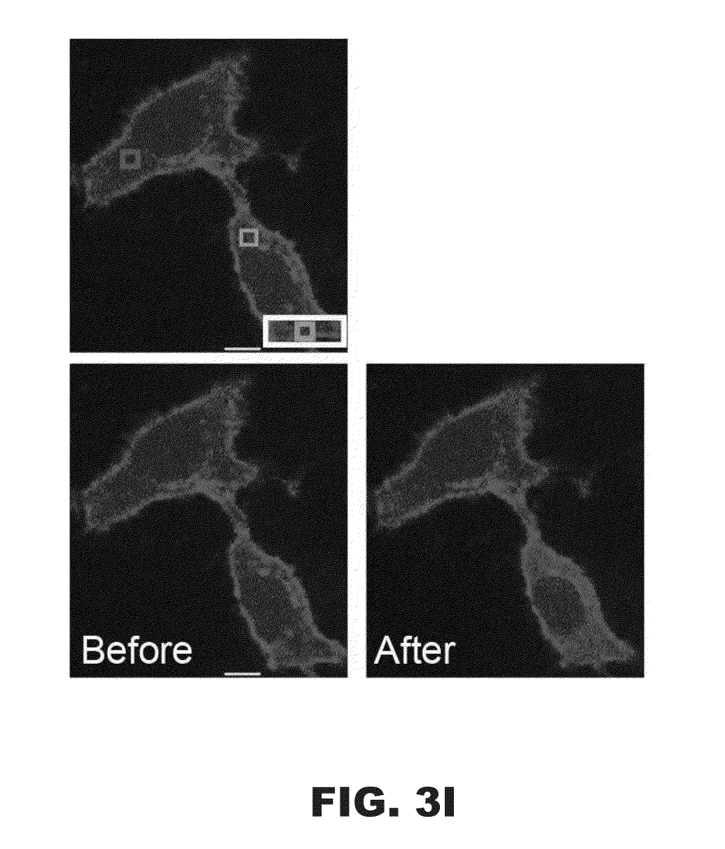

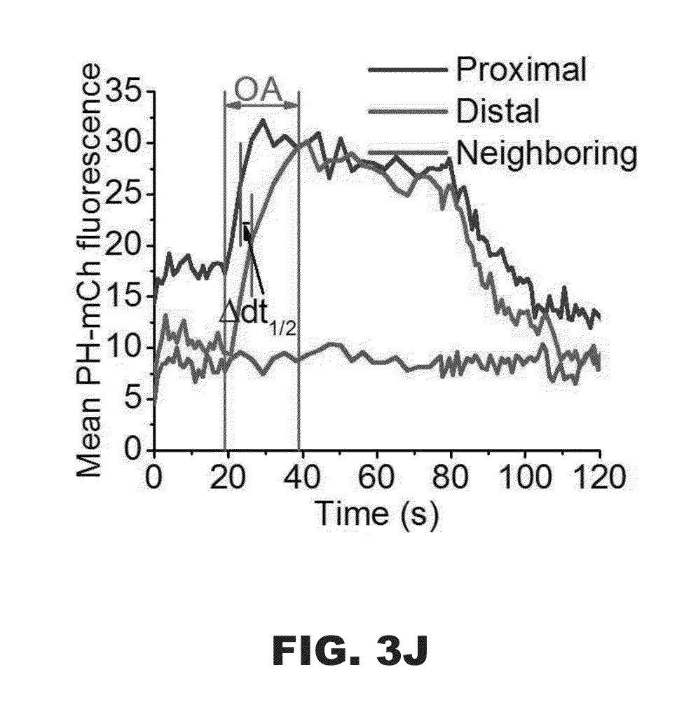

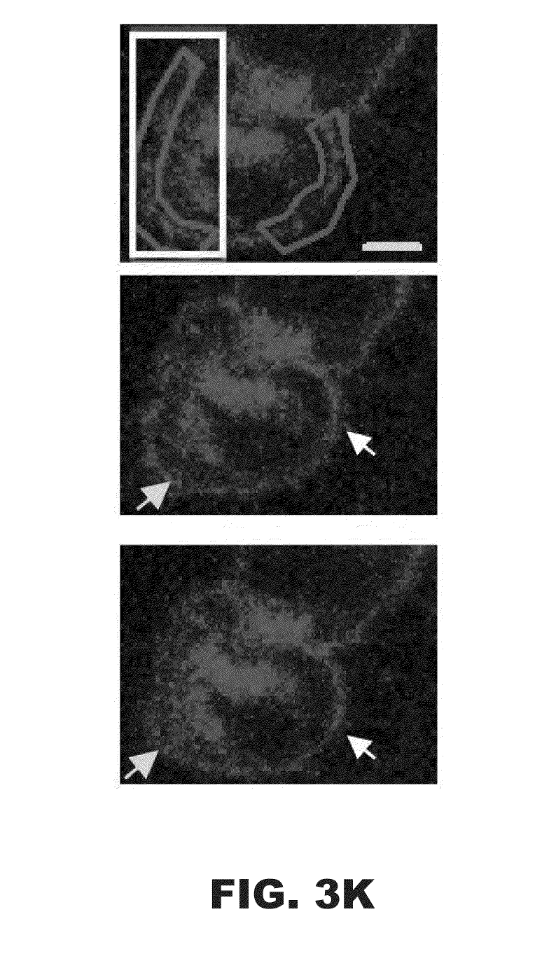

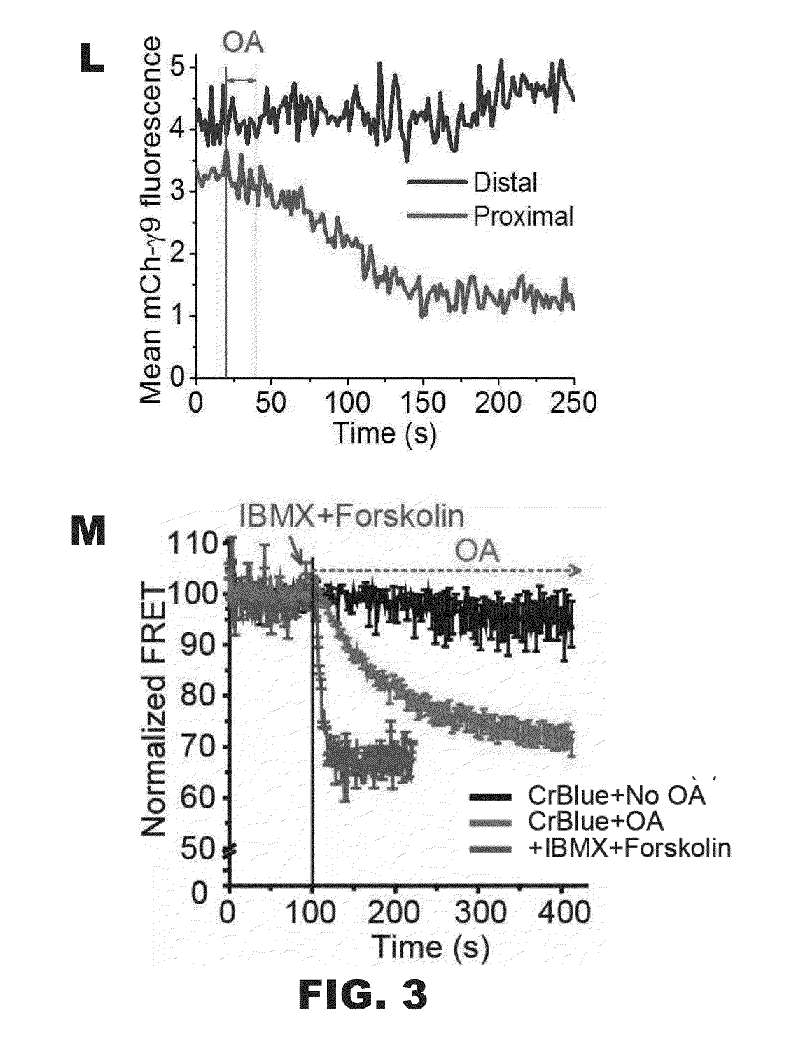

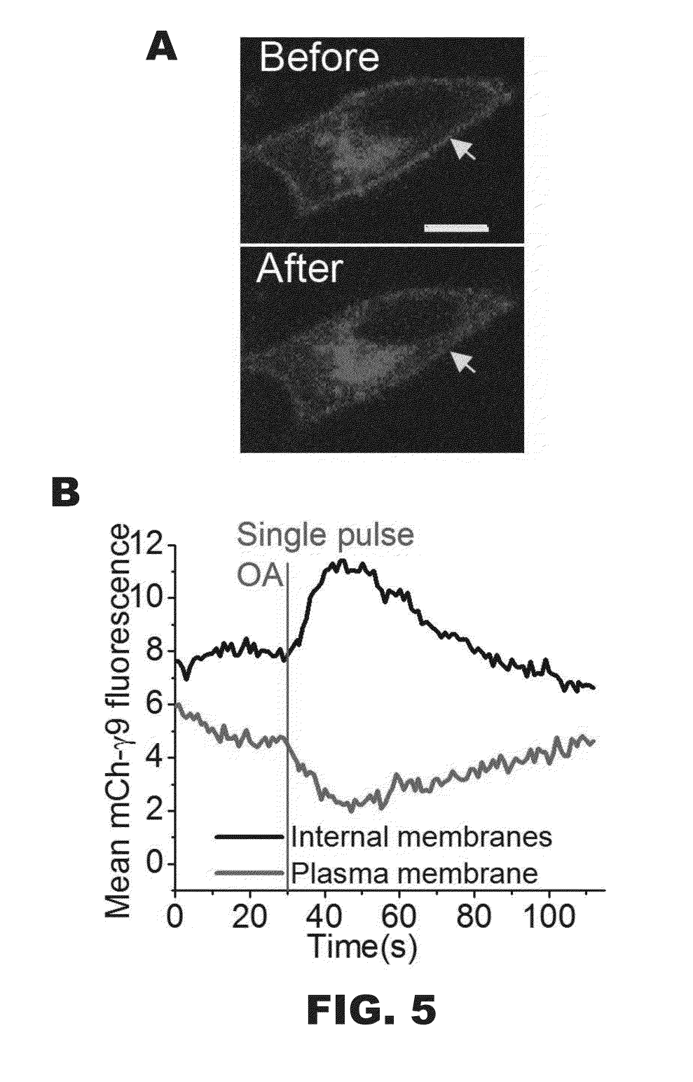

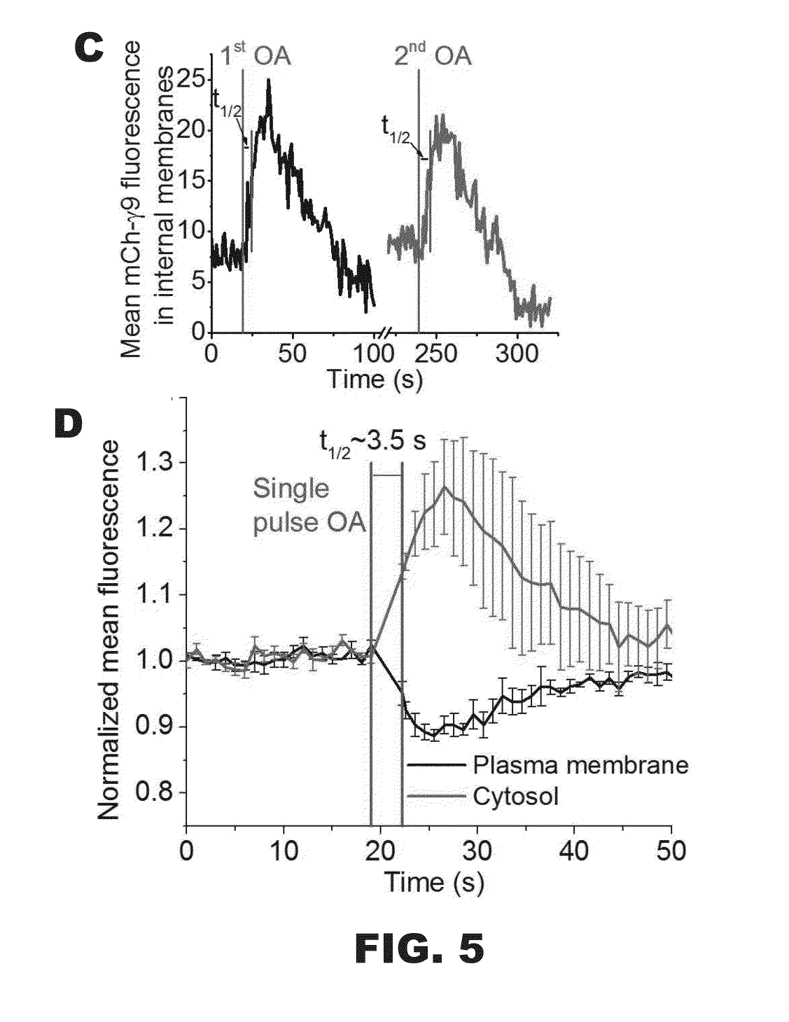

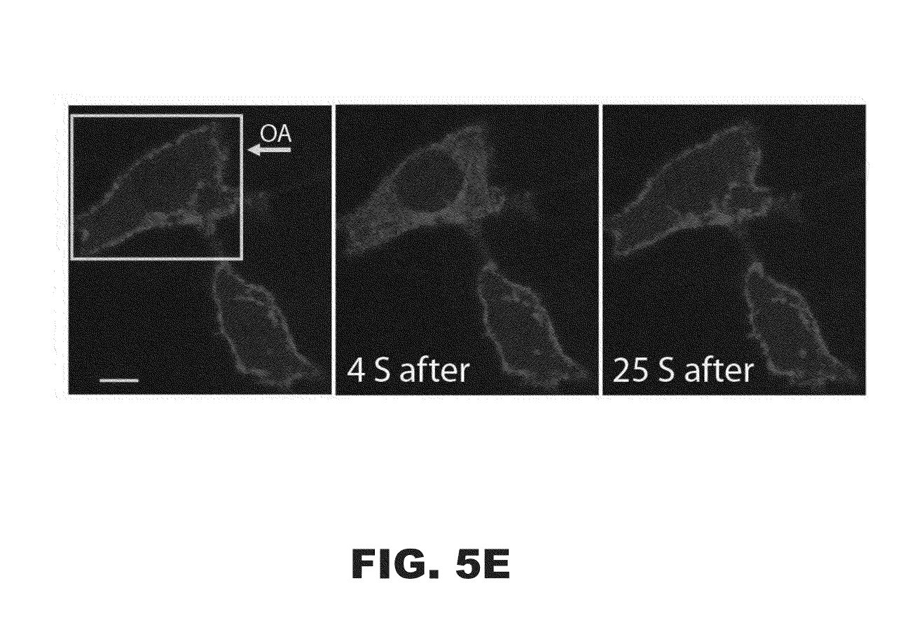

FIG. 3 depicts diagrams, images, and plots describing optical triggers to achieve confined Gi/o, Gq and Gs activation in a cell and simultaneously monitoring the cellular response. A, Imaging basal activity, spatially confining GPCR activation and imaging the resultant response by using spectrally separated wavelengths of light for imaging and opsin activation. Orange and blue—spectrally distinct laser beams. Yellow—selective opsin activation. Green—localized signaling activity. B, Screening for spectrally selective opsins. Opsins activated globally at all specified wavelengths were discarded. C and D, Localized bOpsin activation (white box, 445 nm, 5 μW, single pulse) (blue ROI—activated proximal region, green ROI—unactivated distal region) in HeLa cells (n=7). Plots show intensities in the ROIs. E, Optically induced activation and deactivation kinetics of bOpsin (n=10). F, Repeated bOpsin activation. Recovery was allowed for 1 min before each activation (n=20). G and H, Restricted OA (yellow box) of bOpsin (pulses at 5 s intervals) resulted in localized PIP3 production at the proximal region of a RAW264.7 cell (black plot) and reduction at the distal region (red plot). I and J, Localized melanopsin activation (white box) (488 nm, 27 μW) induced confined IP3 production. Plots show intensity changes in ROIs in image (n=7) (see also FIG. 5). K and L, Localized CrBlue activation (white box) induced spatially restricted mCh-γ9 translocation (red ROI, yellow arrow—activated proximal region, blue ROI and white arrow—unactivated distal area) in HeLa cells (n=7). M, Optical activation of CrBlue (every 5 s) in HeLa cells induced FRET changes in GFP-Δ-epac-mRFP cAMP sensor [GR(488/565)/GG(488/515] (red) (n=6). Control cells (black) were similarly imaged without optical activation. To check the sensor functionality, FRET changes in the GFP-Δ-epac-mRFP cAMP sensor were examined in HeLa cells (green plot) by adding 25 μM Forskolin and 100 μM phosphodiesterase inhibitor, IBMX (final concentrations) at 100 s. Error bars: Mean±SEM.

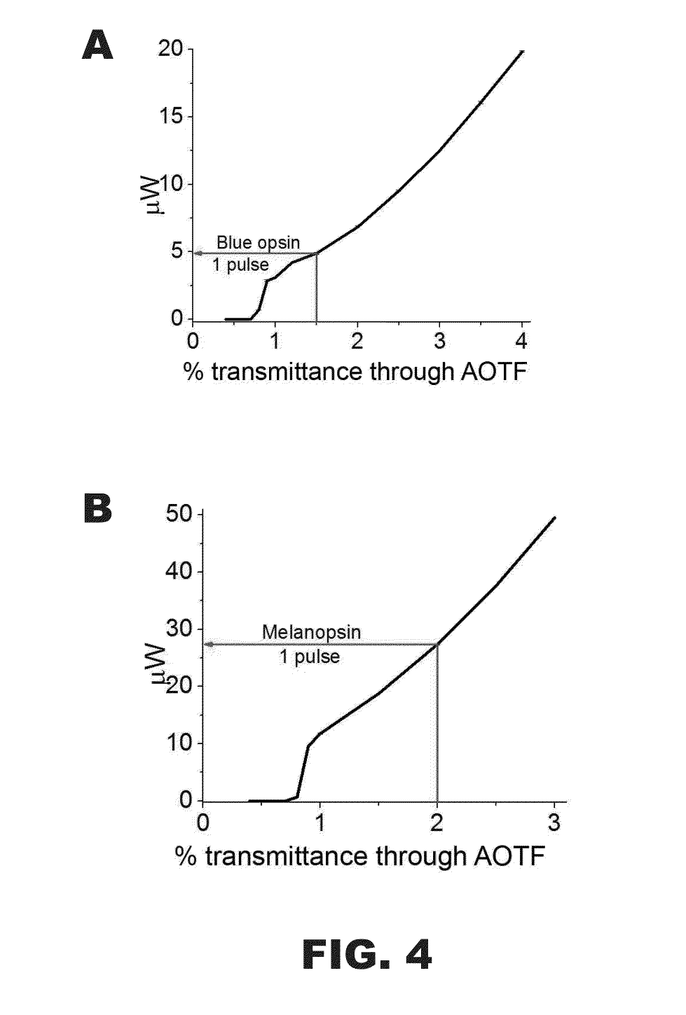

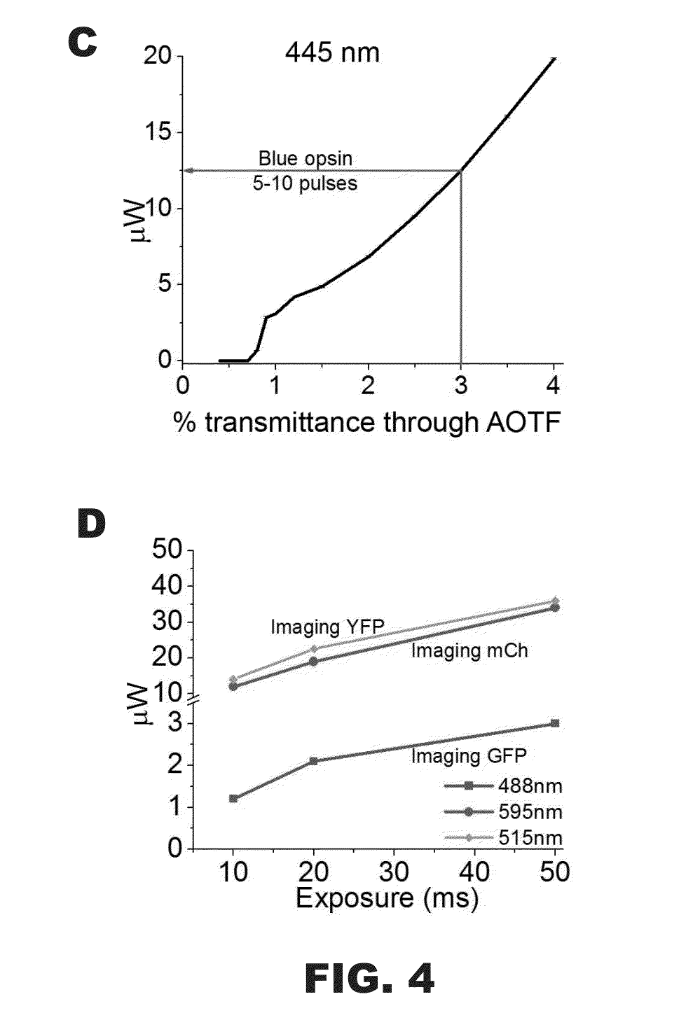

FIG. 4 depicts calibration plots of laser power to be used for confined opsin activation and simultaneous continuous global imaging. A-C, Characterization of optical input beams for spatially confined Gi/o, Gq and Gs activation using titrating the laser power with the Gγ9 translocation induction ability. The laser power (5 μW) for confining OA was measured using a light meter (Ophir Nova II). The red arrow indicates the minimum intensity required to observe detectable translocation of FP-Gβγ9. The intensity was plotted as a function of percentage laser intensity (% laser transmittance through the AOTF-Acousto-Optic Tunable Filter by varying the voltage applied). D, Determination of appropriate laser intensities for imaging signaling activities using GFP, YFP and mCh induced by localized blue opsin, melanopsin and CrBlue activation without evoking the global opsin activation. The following combinations of lasers were used to image the elicited response with the specific opsin: bOpsin—488, 515, 595 nm, Melanopsin—595 nm, CrBlue—488, 515, 595 nm. Error bars: standard deviation.

FIG. 5 depicts images and plots showing localized activation of Gq signaling by melanopsin. A and B, Single pulse OA of melanopsin induced mCh-γ9 translocation. Plot shows increase in mCh-γ9 in intracellular membranes and decrease in the plasma membrane. C, Repeated activation (2 min apart) of melanopsin induces repeated translocation of mCh-γ9 (t½=˜6 s, n=6). D and E, OA of melanopsin induces PH domain translocation in HeLa cells. A HeLa cell expressing melanopsin and PH-mCh was optically activated (entire cell, yellow box) with a single pulse of light. PH-mCh translocated to the cytosol (image-4 s). There was complete reversal of PH-mCh to the plasma membrane over time (image-25 s). Plot shows mCh intensity changes in the plasma membrane and the cytosol. (n=4).

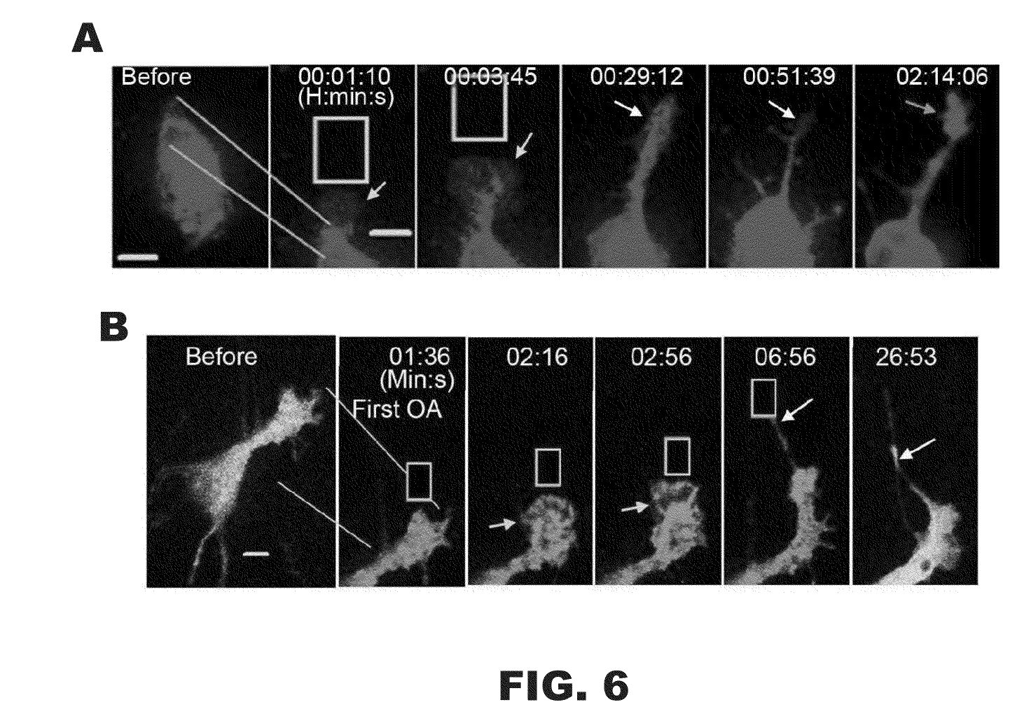

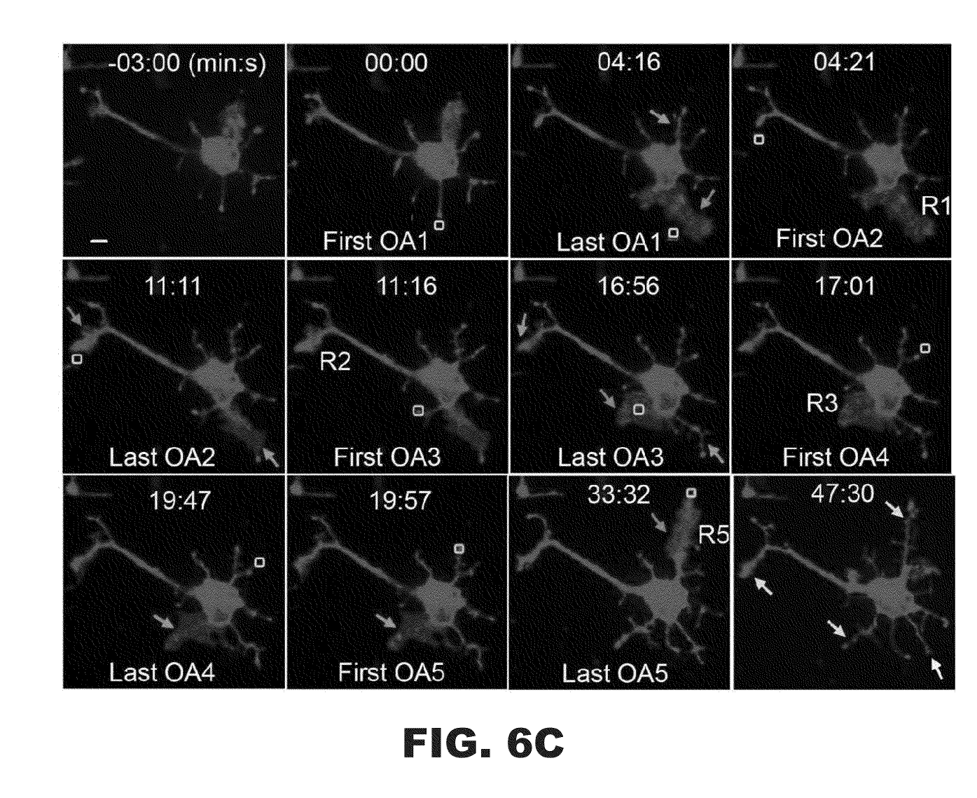

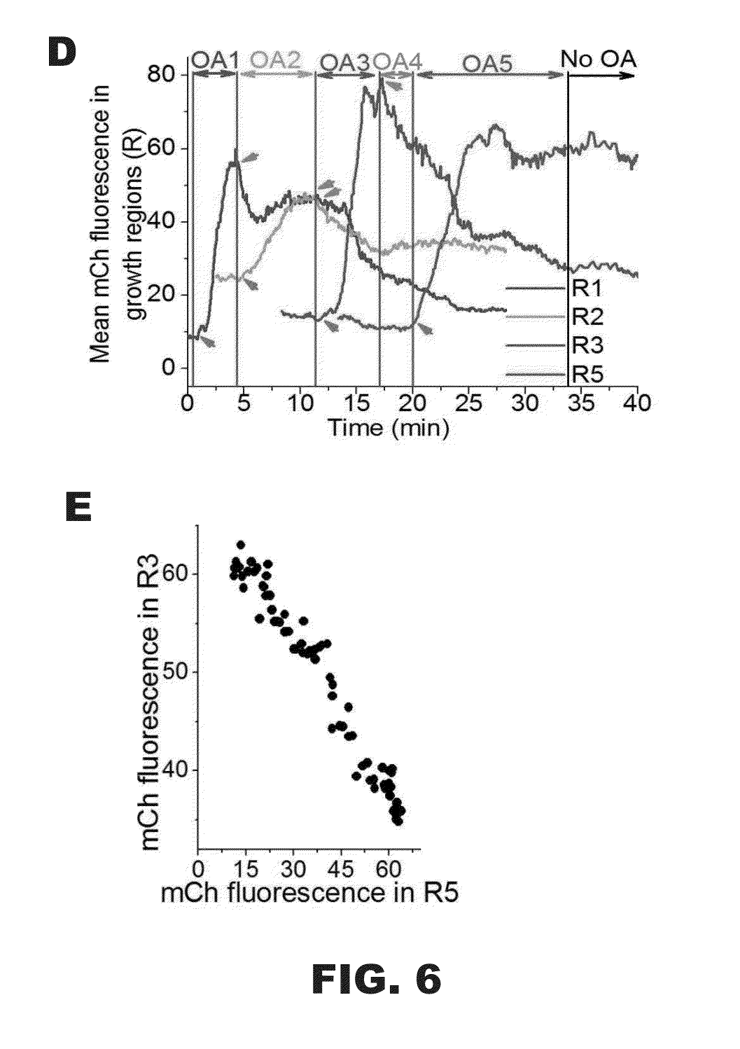

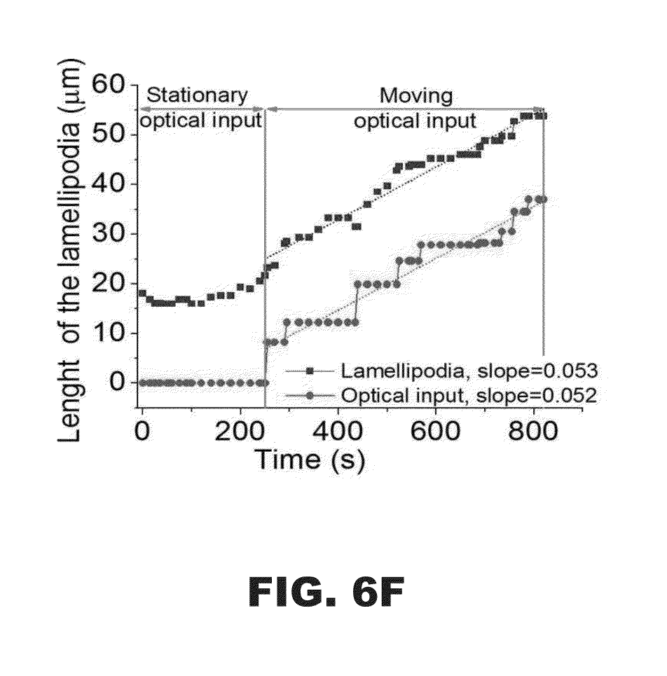

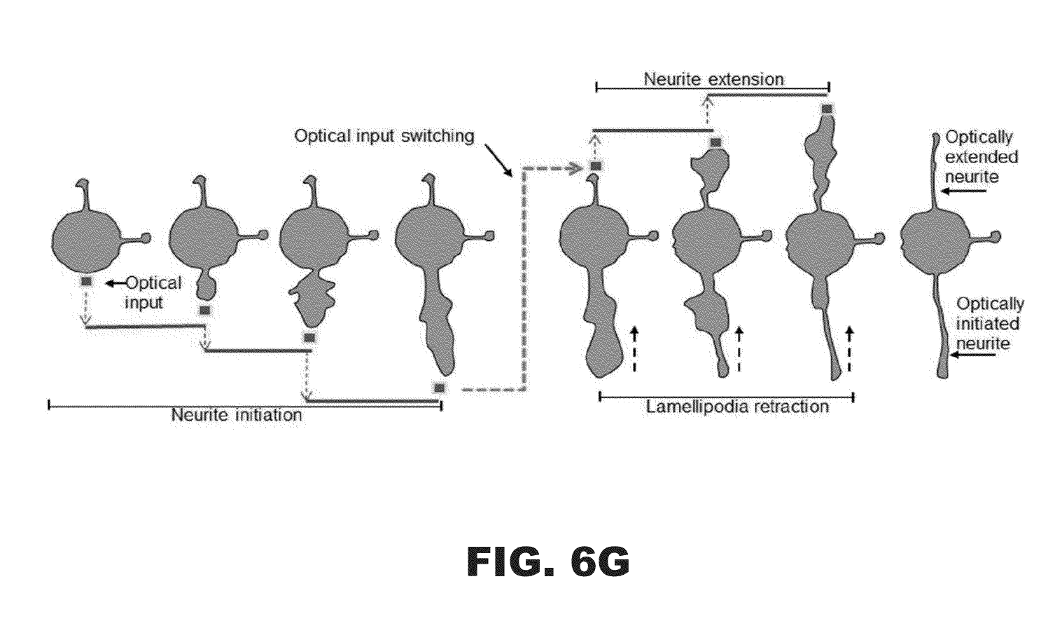

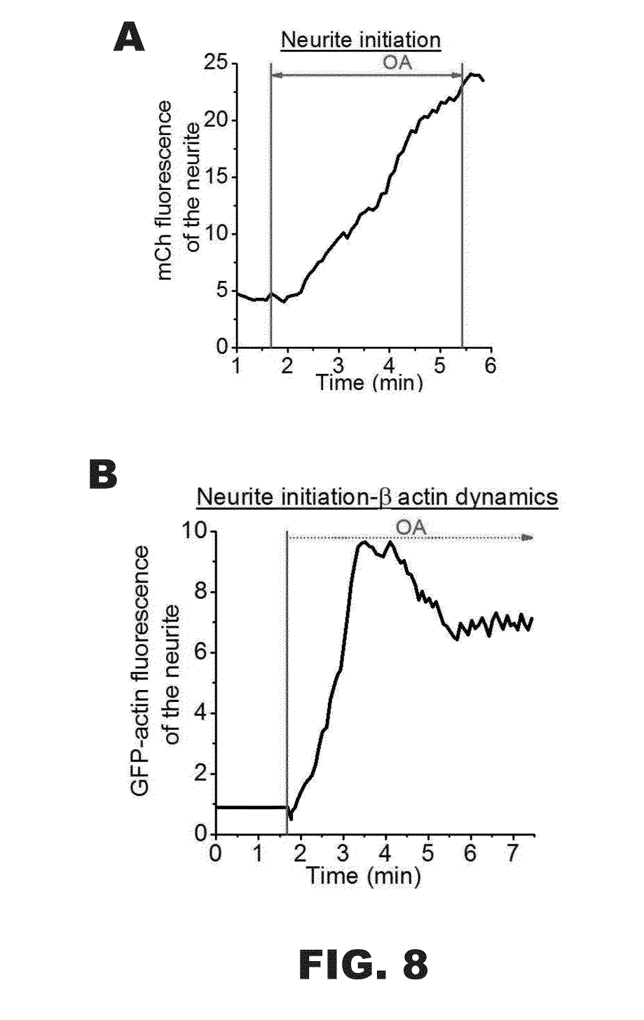

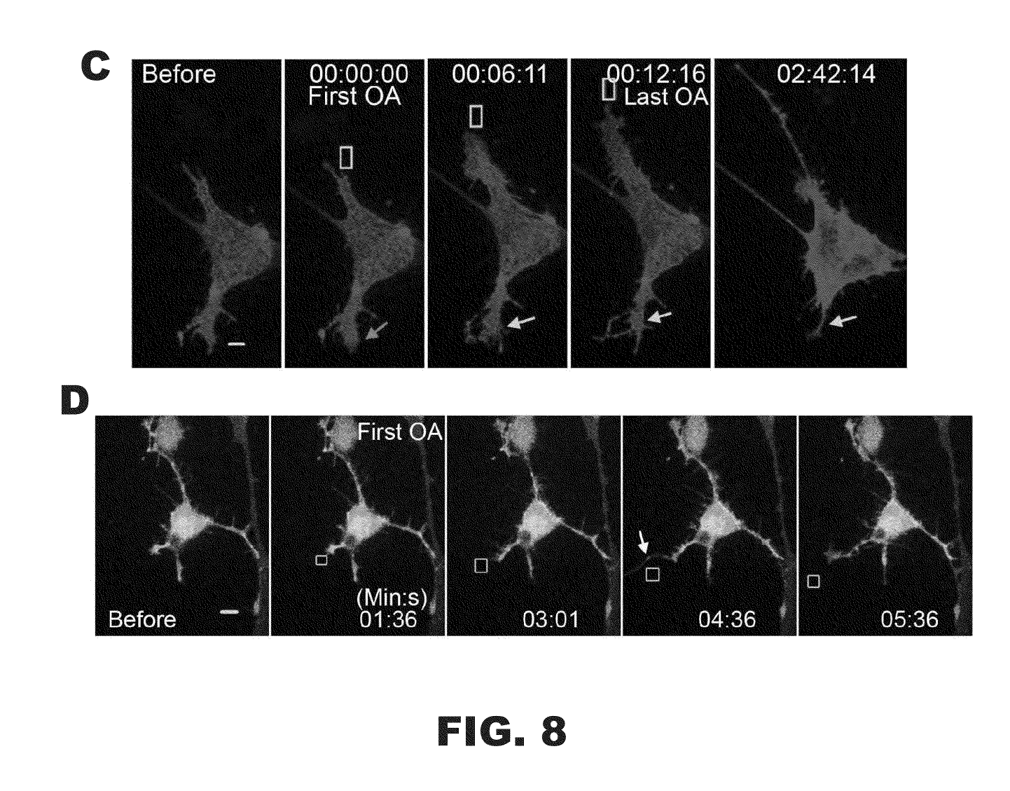

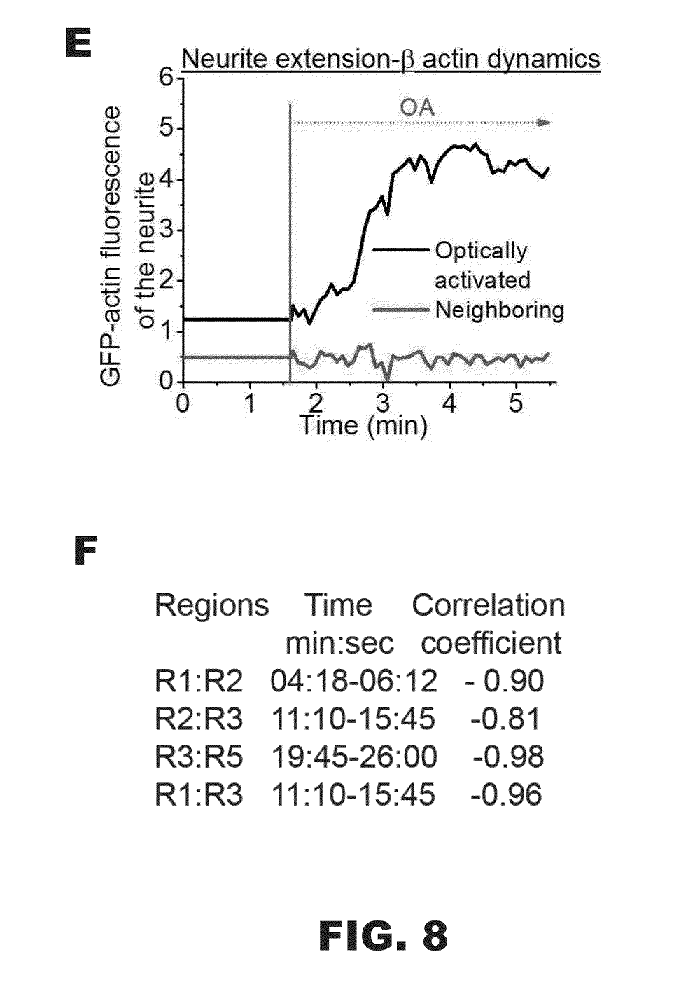

FIG. 6 depicts images, diagrams and plots showing programming neuronal differentiation through spatiotemporally discrete optical inputs to bOpsin. A, OA (yellow box, 445 nm, 5 μW, pulsed at 5 s intervals) elicited neurite initiation in rat hippocampal neurons expressing bOpsin-mCh. Selected area of neuron is shown (yellow lines). Lamellipodia (yellow arrows) consolidate into a neurite (white arrows) with a growth cone (green arrow) (n=6). B, OA induced actin cytoskeleton remodeling in neuron expressing bOpsin-mCh and mGFP-actin. Both mCh and GFP images were captured before and after OA and overlaid (extreme left and right). Actin rich lamellipodia (yellow arrow) later consolidated into a neurite (white arrow) (n=7). C, Optically refashioning neuronal differentiation by sequential OA (yellow box) of single neurite tips. Activation of neuron expressing bOpsin-mCh extended lamellipodia (blue arrows) simultaneously lead to retraction of distal growth area (green arrows) (R1-R5) (see also FIG. 8F). Comparison of the last image with the basal image shows that three neurites have been extended (yellow arrows) and a new neurite created (white arrow). D, Plots represent lamellipodia extension-retraction dynamics in C. E, Correlation between the selected lamellipodia growth and retraction. F, Single neurite growth dynamics in response to selective optical input varying in time and space. G, Optical input functions can be designed to reprogram neurite patterning.

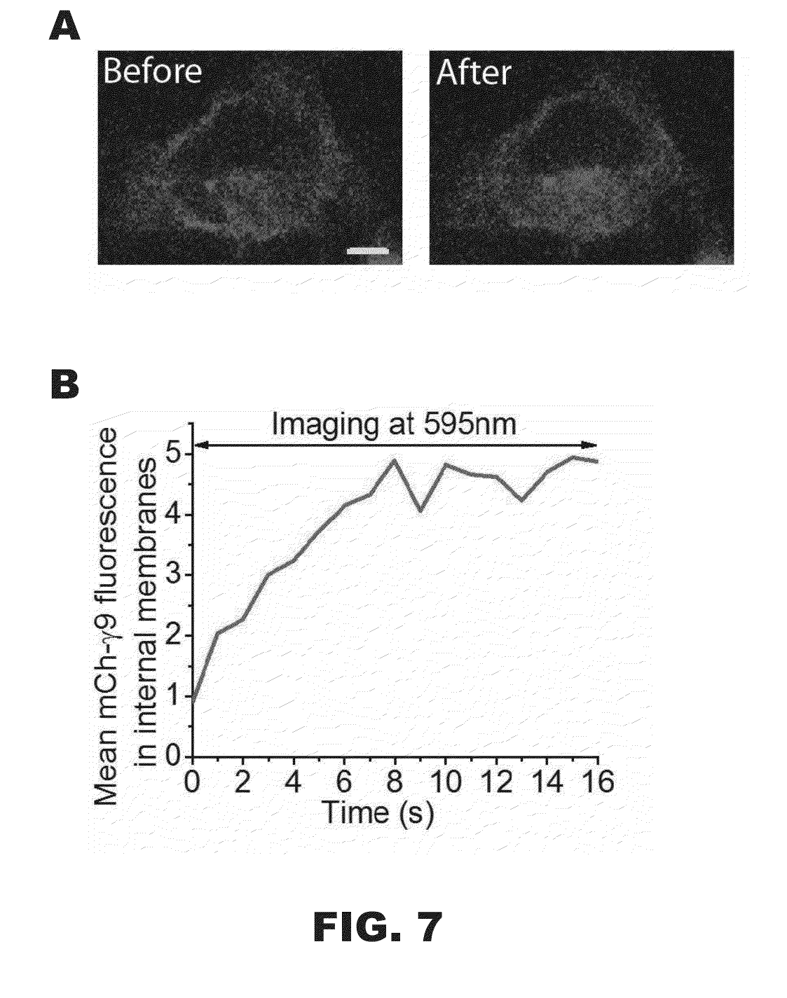

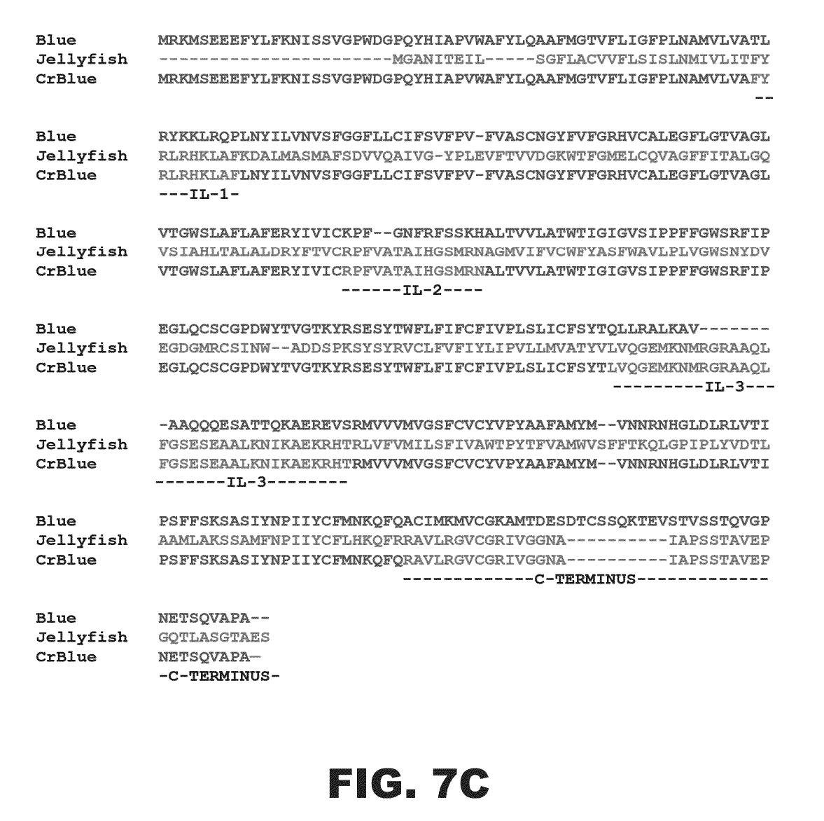

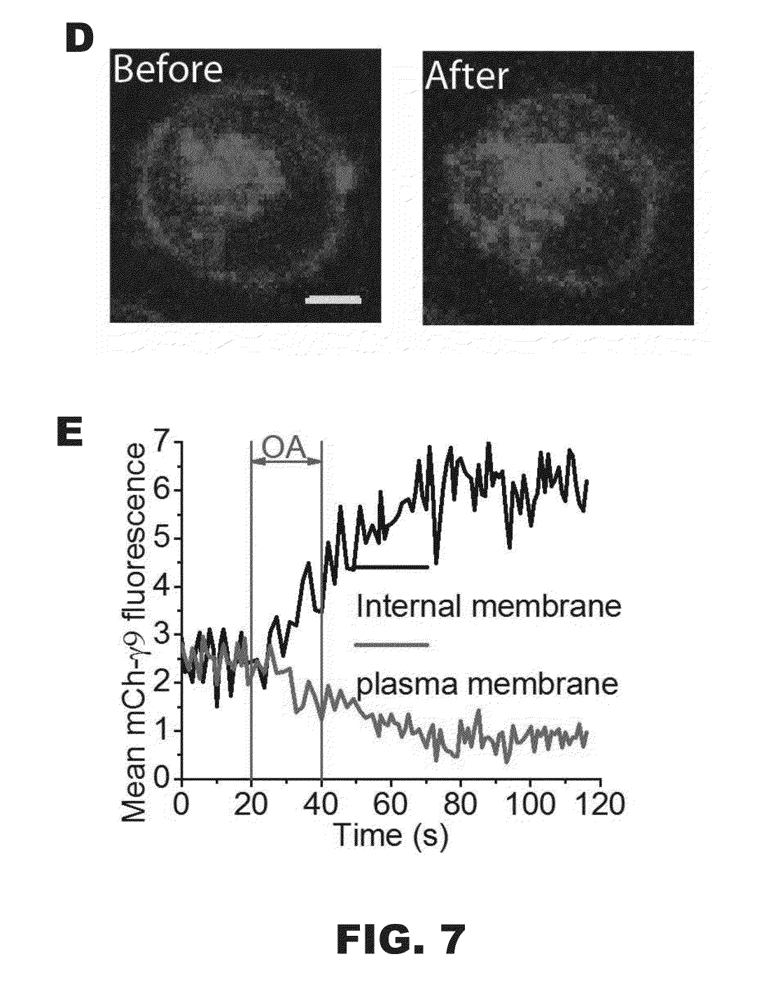

FIG. 7 depicts images, plots and a sequence comparison regarding engineering a spectrally selective chimeric opsin for localized Gs signaling. A and B, Gs coupled jellyfish opsin activation induced mCh-βγ9 translocation in HeLa cells during imaging mCh (n=10). Plot shows increase in mCh-βγ9 in intracellular membranes without a base line. C, Design of spectrally selective Gs coupled opsin (CrBlue, SEQ ID NO: 3) using extracellular and retinal binding transmembrane regions of blue opsin (red, SEQ ID NO: 1) that are responsible for its spectral tuning and Gs heterotrimers interacting with cytosolic loops of jellyfish opsin (green, SEQ ID NO: 2). IL-Intracellular loops. D and E, CrBlue activation induced mCh-γ9 translocation in HeLa cells (n=8). Plot shows that in contrast to jellyfish opsin, the basal state imaging does not activate CrBlue.

FIG. 8 depicts plots and images showing optical control of neurite initiation and extension in rat hippocampal neurons. A, OA induced lamellipodia formation dynamics of neurite initiation during neuronal symmetry breaking (FIG. 6A). B, β-actin dynamics during a OA induced neurite initiation. C, OA induced neurite extension in a precursor expressing bOpsin and DenMark-mCh (dendritic marker). During OA (yellow box) of selected region of neuron, spontaneously growing lamellipodia at the opposite end of the neuron (green arrow) retracted (yellow arrows). D, Actin remodeling during OA stimulated neurite extension in a neuron expressing bOpsin-mCh and mGFP-actin. Formation of actin rich filopodia (white arrow) (n=5). E, Actin dynamics during OA stimulated neurite extension. F, Correlation coefficients between optically induced lamellipodia growth and corresponding distal growth retraction.

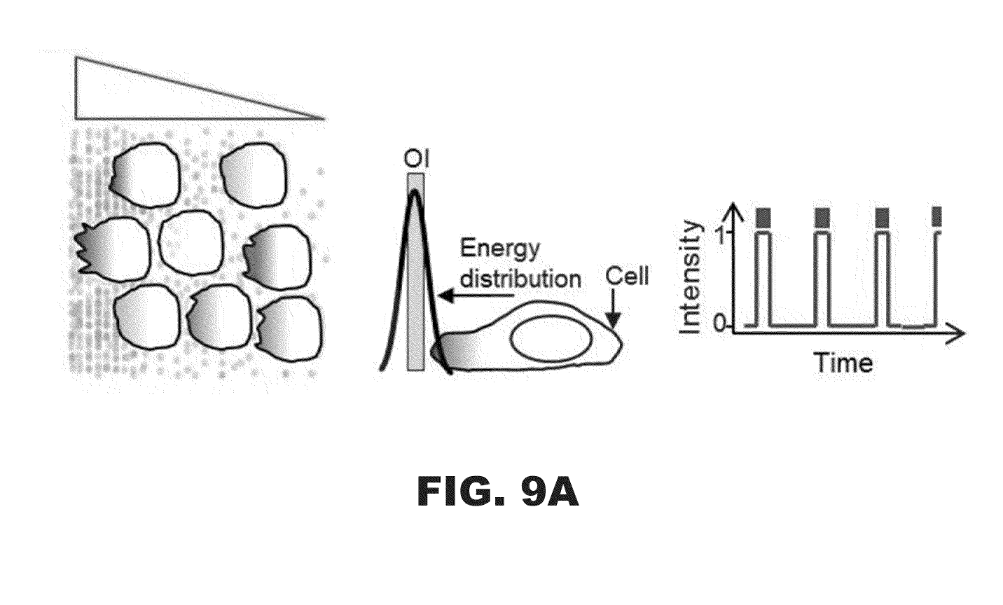

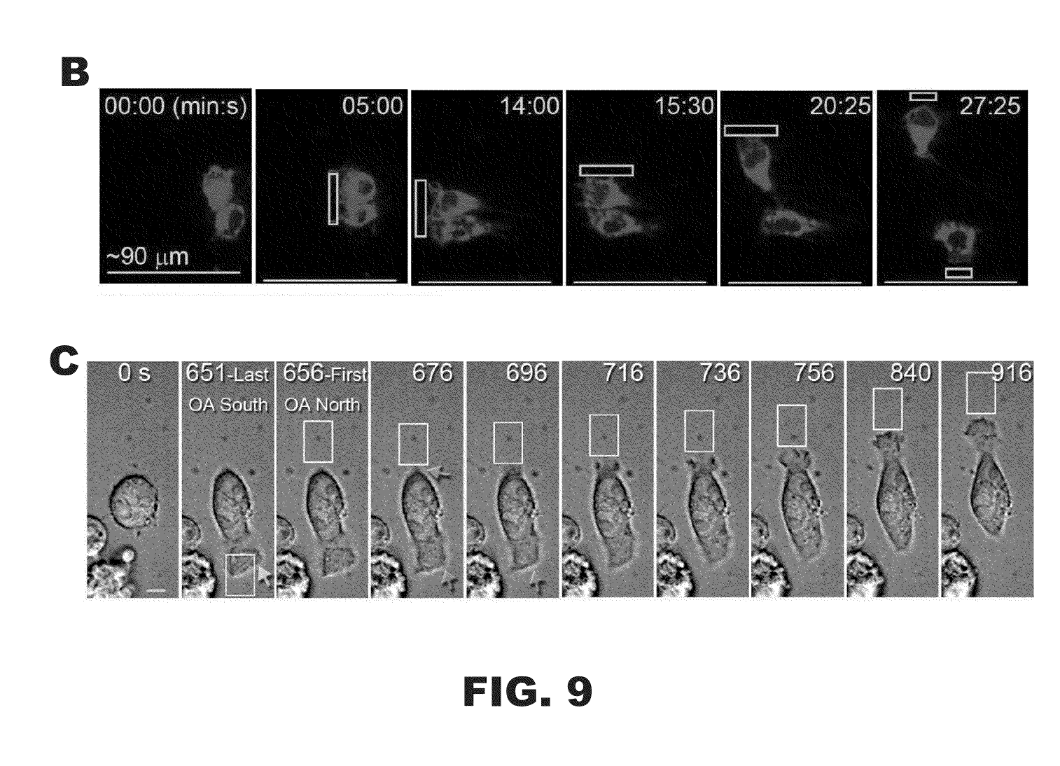

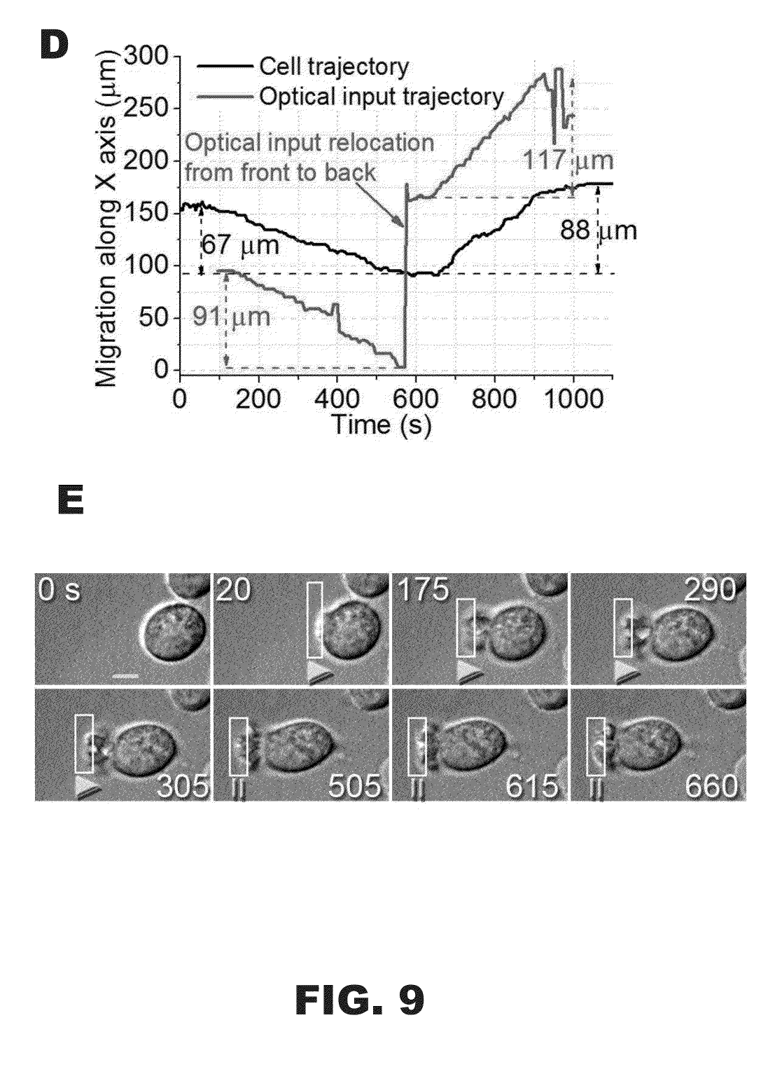

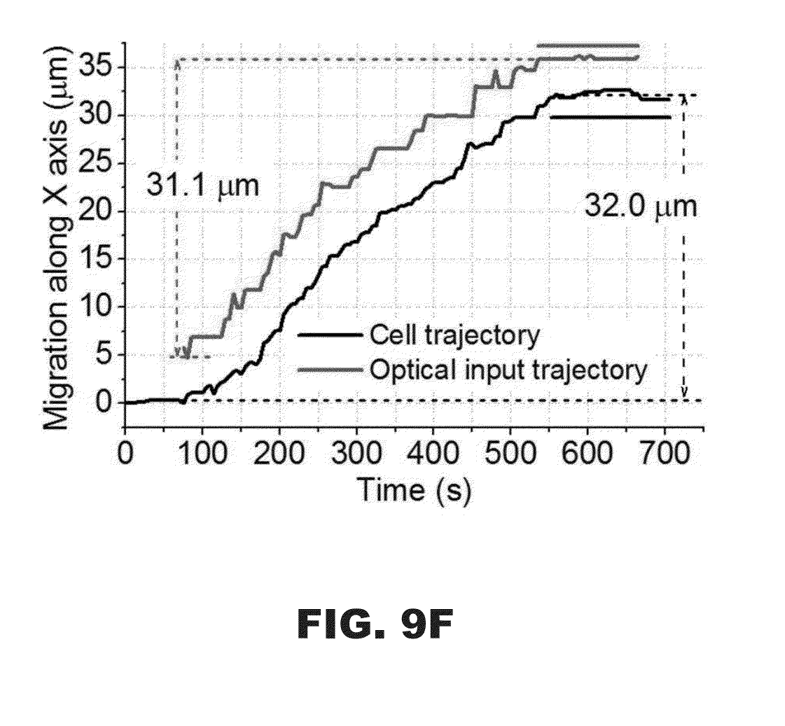

FIG. 9 depicts images, diagrams and plots showing that optically induced signaling asymmetry controls directionally sensitive immune cell migratory behaviors. A, Migration induced by a diffusible gradient and by optical activation. (a) Cells migrate toward the higher concentration of chemoattractant molecules. Blue triangle depicts gradient. (b) Optical input location, area, intensity, number of pulses, and intervals between pulses are designed to evoke spatially asymmetric signaling activity. B, Confined OA (yellow box) of bOps-mCh directs RAW cell migration (n>40). C, Reversal of RAW cell migration by OA of bOpsin (white box). Yellow arrow: growing lamellipodia. Orange arrows: lamellipodia retraction. Green arrow: lamellipodia appearance at new front. D, Directional coupling of cell and optical input trajectories (X axis) (n=5). E and F, Continuous monitoring of initiation of migration, maintenance of migration and adaptation by controlling the optical input. In (E) [symbol] =moving optical input, II=stationary optical input. In (F) Plot shows optical input (red) and cell (black) trajectories.

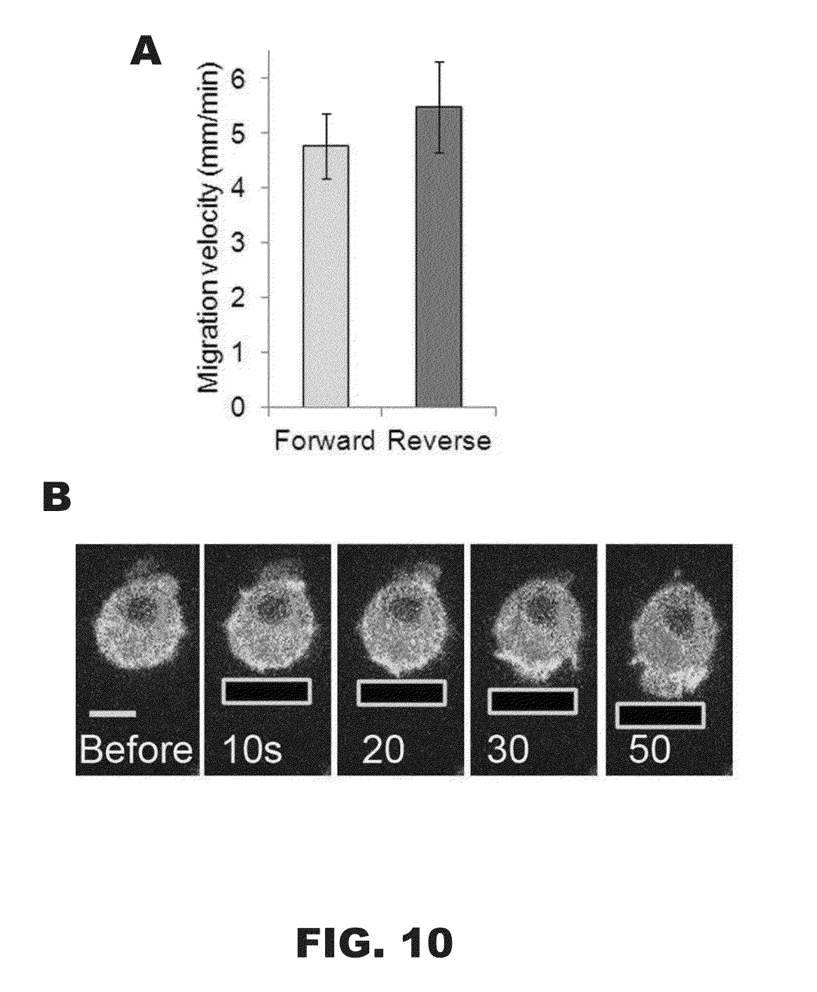

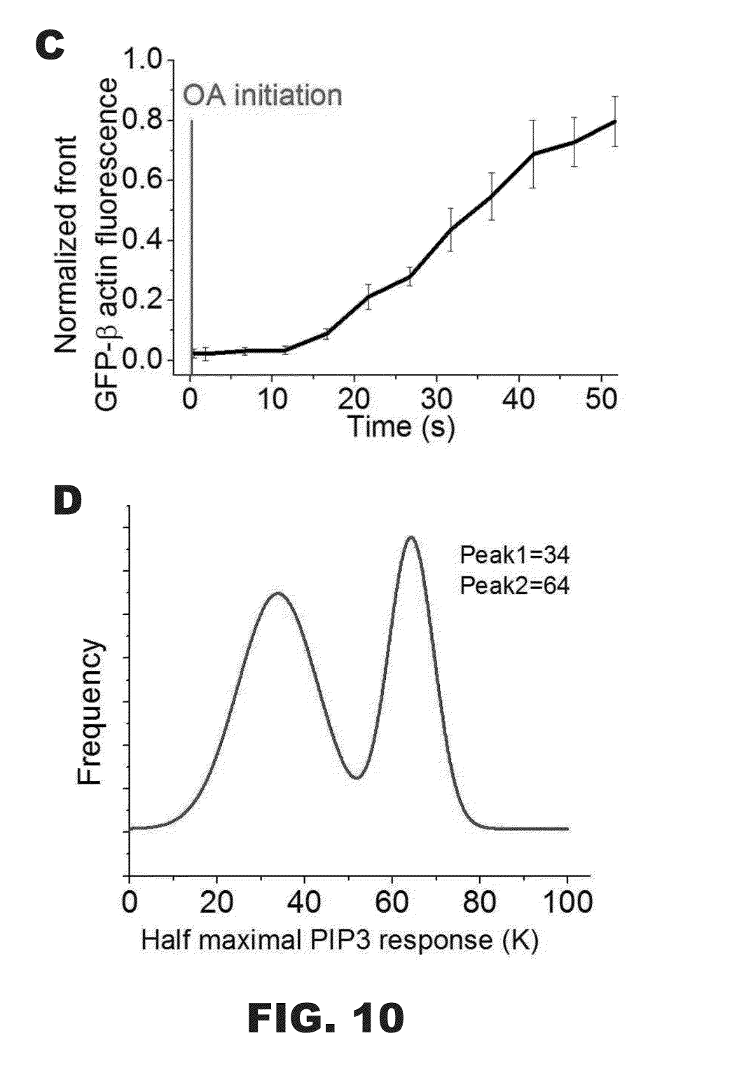

FIG. 10 depicts 5 images and three plots showing analysis of optically stimulated cell migration. A, Mean forward and reverse velocities of RAW cells during OA induced cell migration (n=5). B, Optical induced actin rich lamellipodia formation in a RAW cell with bOps-mCh (red) and mGFP-actin (green). C, Normalized front GFP-β actin fluorescence over time. D, Statistical distribution analysis of half maximal PIP3 response in migrating RAW 264.7 cell population.

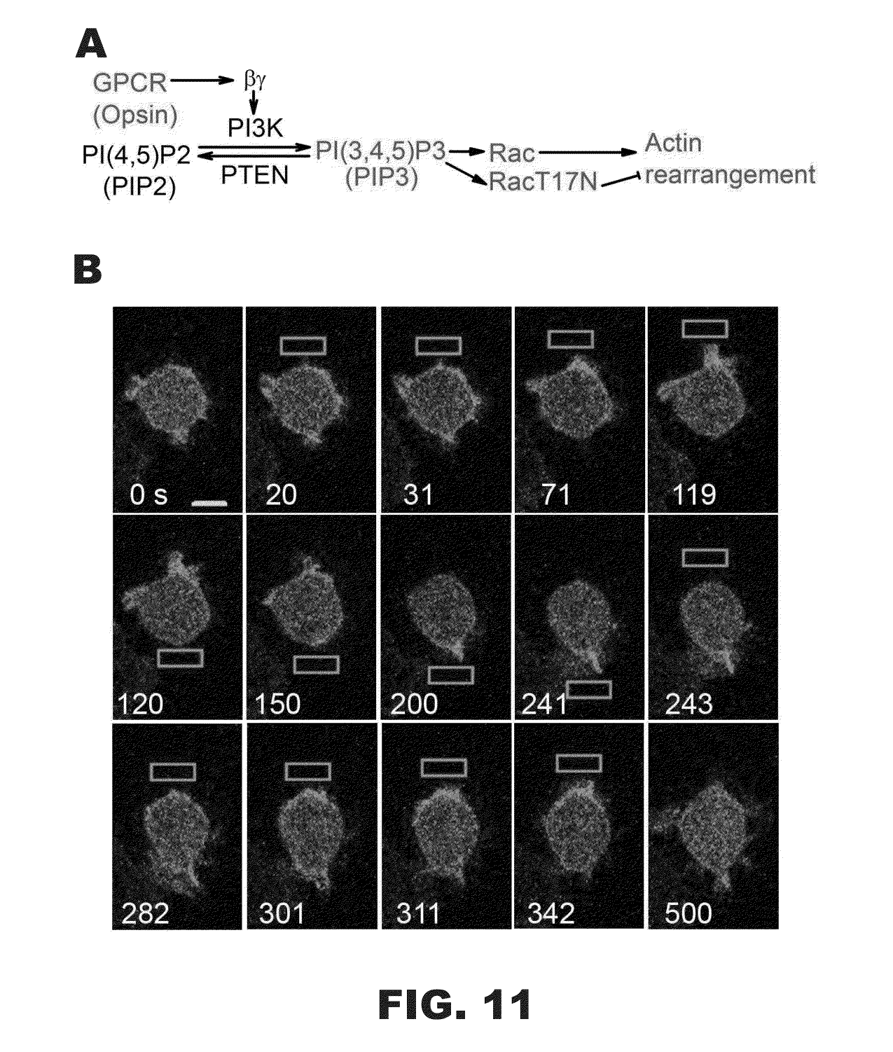

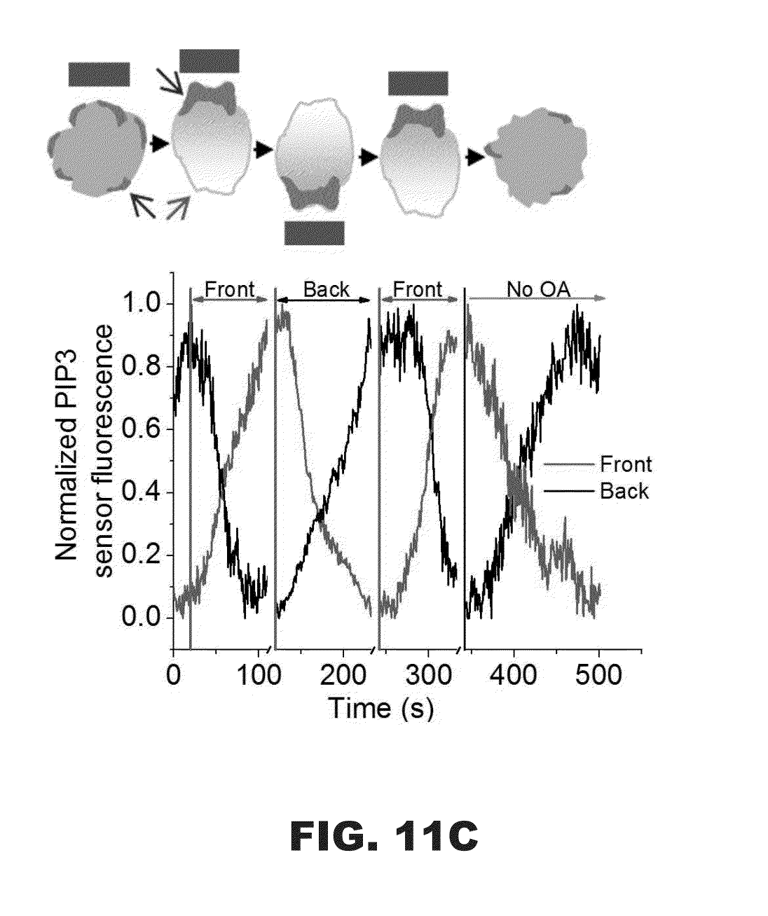

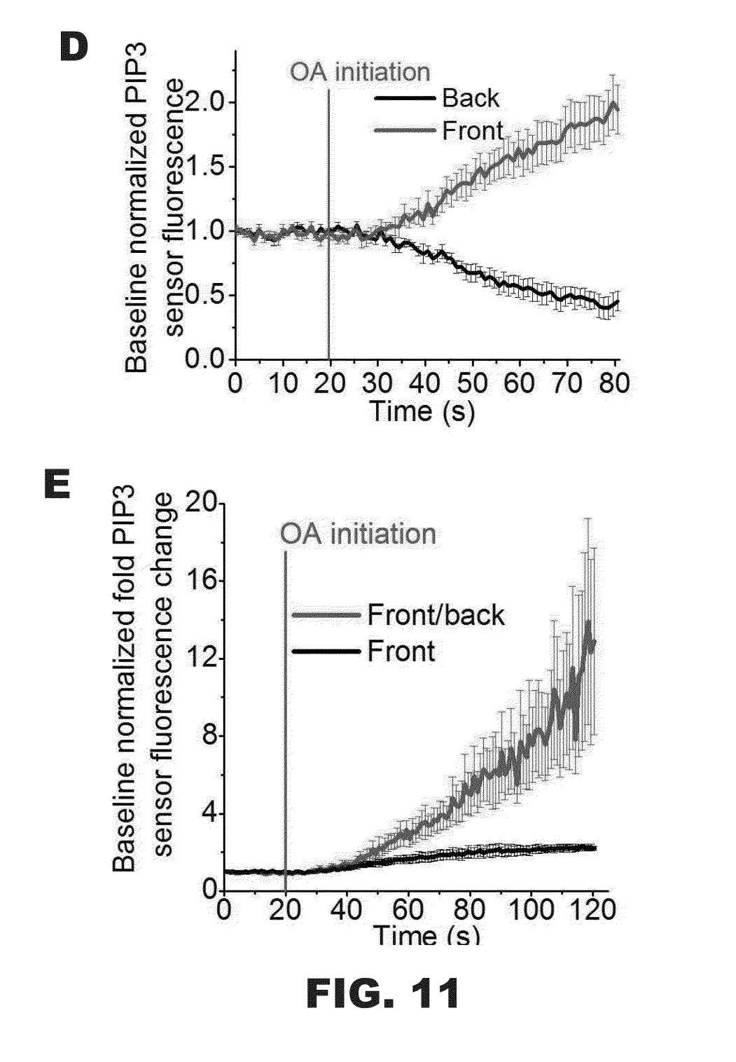

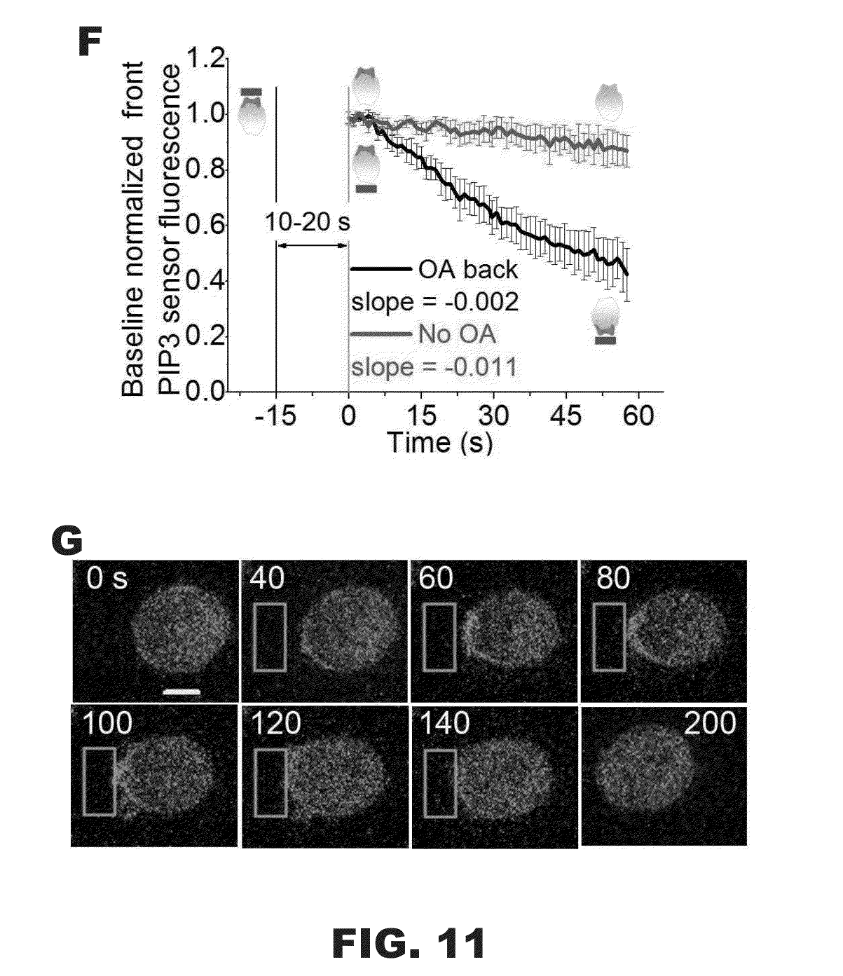

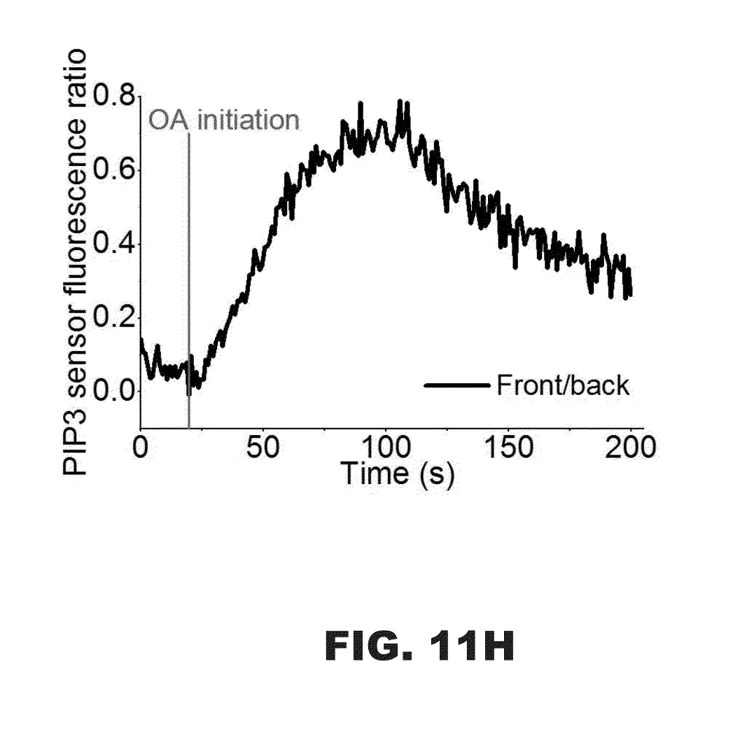

FIG. 11 depicts images, diagrams, and plots describing monitoring PIP3 dynamics and cell migration in response to multiple spatially and temporally variant optical inputs. A, Signaling pathway involved in GPCR mediated cell migration. B, Repeated switching of optical input (1 s intervals) and monitoring PIP3 gradient in a single RAW cell expressing bOps-mCh and PIP3 sensor, Akt-PH-GFP. Green box: optical input. C, Design of experiment. Blue arrow: PIP3 presence; red arrow: absence of detectable PIP3. Purple box: Optical input. Plot below shows front (red) and back (black) PIP3 sensor fluorescence intensity changes corresponding to the states depicted above. Vertical lines (purple): OA of front or back. Vertical line (black): OA termination. D, Changes in PIP3 sensor mean fluorescence intensity in the front (red) and back (black) after onset of OA (n=5). E, Normalized front (red) to back (black) PIP3 sensor fluorescence intensity ratio during OA of the front. F, PIP3 sensor mean fluorescence intensity at the front on removal of OA (red, n=6) or relocation of OA (black line) to back (black, n=4). Time=0 is the point at which fluorescence starts to decrease (orange line). Cartoons represent OA status and cell state in corresponding plots. Data in B were included in the analysis of D-F. G and H, Continuous monitoring of PIP3 dynamics during adaptation in migrating immune cell (n=8). Error bars: Mean±SEM.

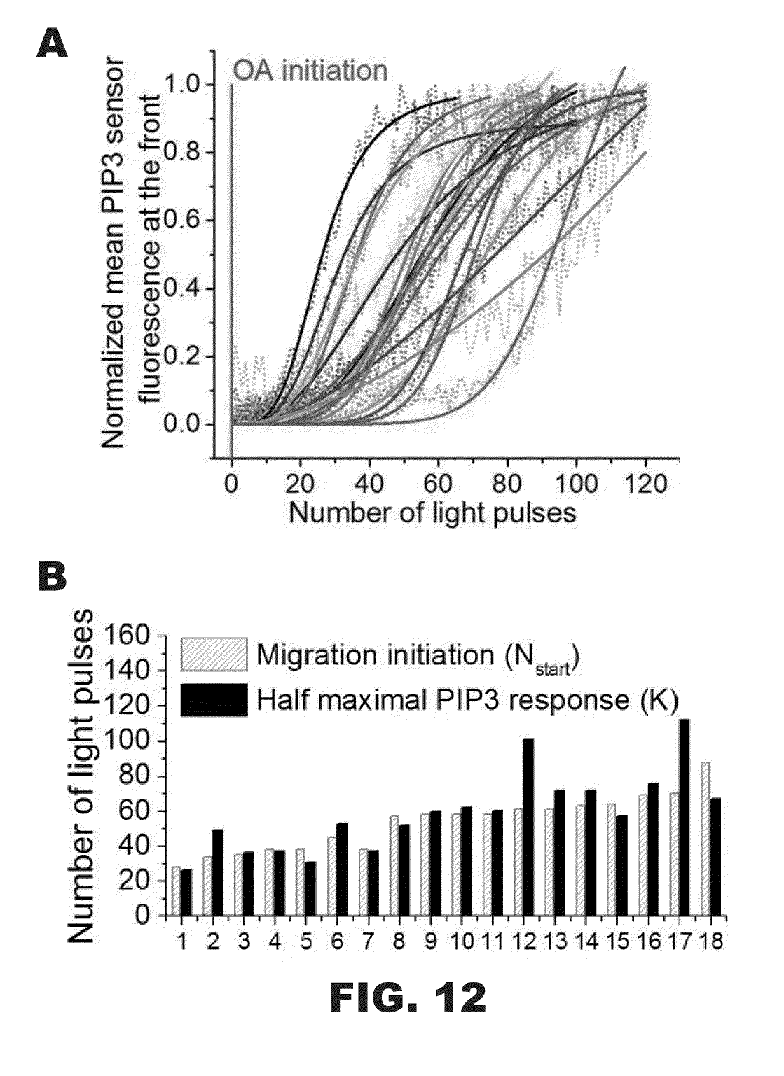

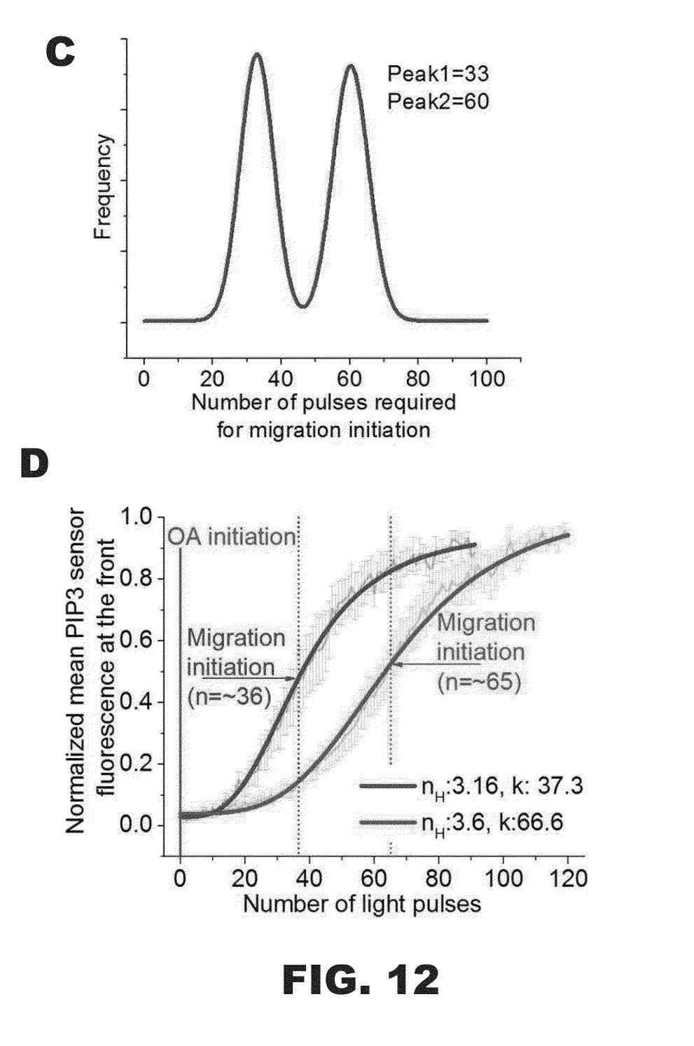

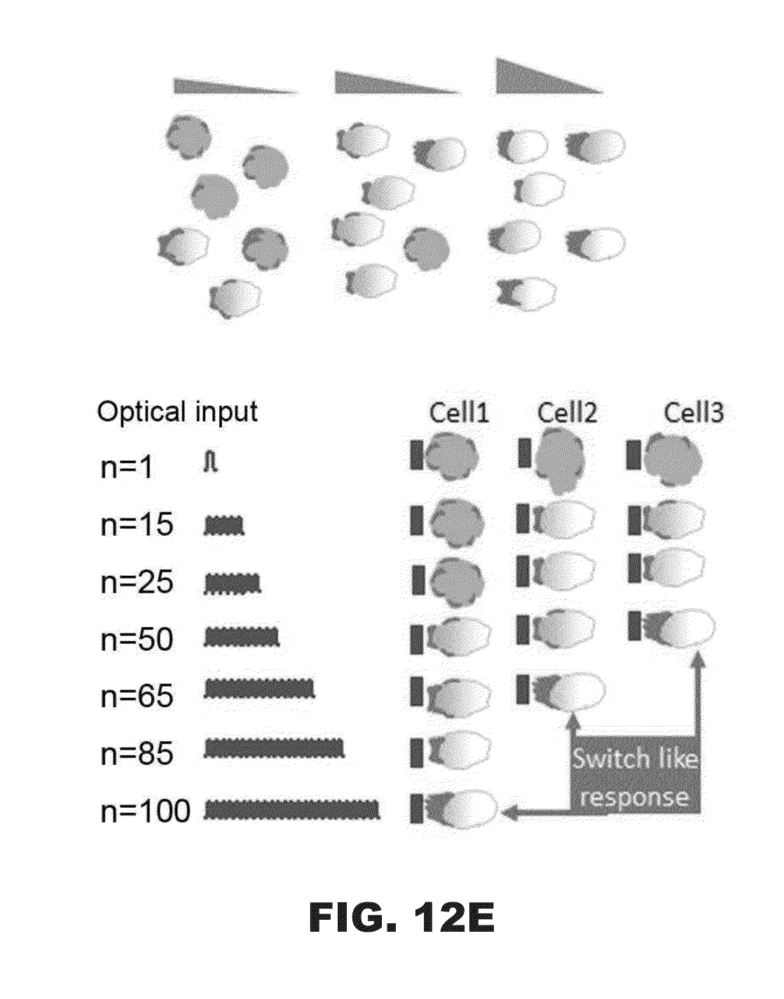

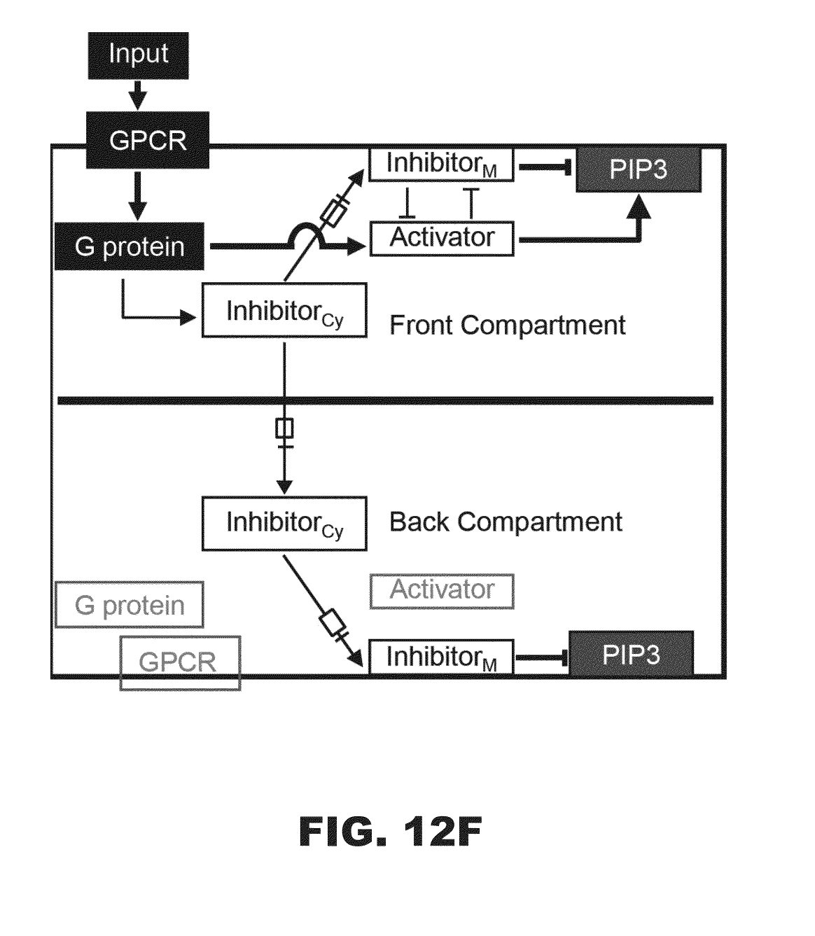

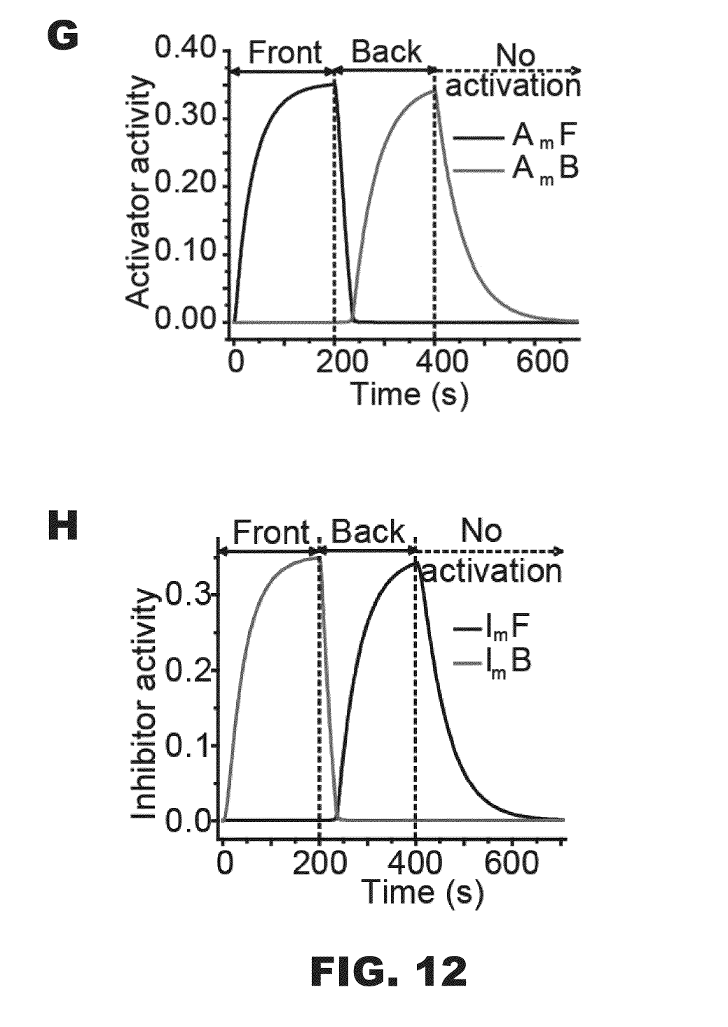

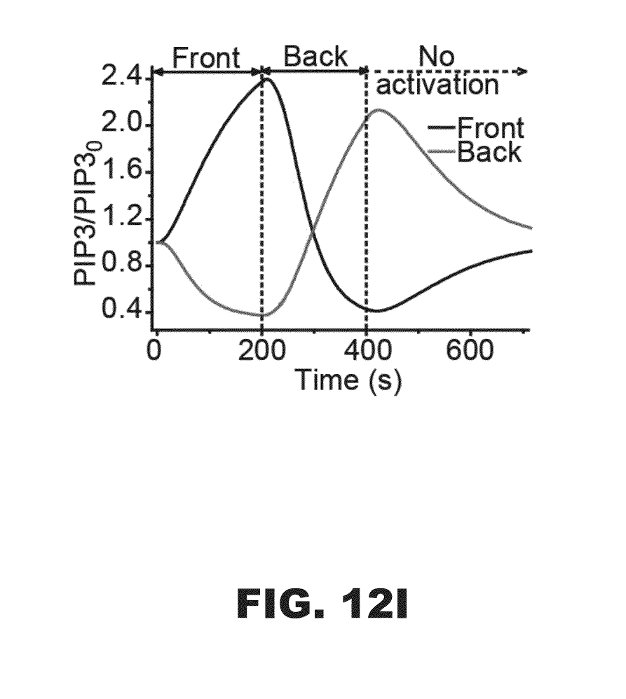

FIG. 12 depicts plots and diagrams showing that mathematical modeling and optical methods reveal systems properties in a single cell. A, Experimentally observed ultrasensitive responses of fractional PIP3 at the cell front to increasing number of light pulses in individual cells (n=18). Dotted lines: experimental data, solid lines: Hill fit (n_{H}: 2.6-7.7). B, Bar chart showing half maximal PIP3 response (K) and input required for migration initiation for individual cells. C, Population analysis of 23 cells above based on input required for migration initiation indicates a bimodal distribution (cut point=46). D, Experimental PIP3 response plots created by grouping two populations based on number of pulses required for migration initiation (Nₛₜₐᵣₜ). Group 1: Nₛₜₐᵣₜ<46, Group 2: Nₛₜₐᵣₜ>46. Light pulses required to reach half maximal PIP3 response (K) are shown. All analysis presented here is with the same or overlapping population of cells. E, In contrast to population analysis using diffusible gradients which masks individual cell behaviors (upper panel), the optical approach developed here identified single cell network properties that govern behavioral responses (lower panel). It helps uncover cell to cell variation and classifies a population on the basis of network properties of individual cells. Error bars: Mean±SEM. F, Two-compartment model for immune cell migration. Simplified schematic representation of the biomolecular pathway at the cell front and back describing PIP3 regulation during immune cell migration. Arrows with a box indicate either recruitment to the membrane or translocation from front to back compartment. cyt, cytosolic; m, membrane bound. GPCR, G protein, and the activator in the back compartment are inactive and presented in gray. G, Simulated activator dynamics in the front (AmF) and back (AmB) compartments of a single cell in response to switching the localized activation from front to back. H, Simulated membrane recruited inhibitor dynamics in the front (ImF) and back (ImB) during corresponding activation switching. I, PIP3 dynamics at cell front and back controlled by the activity of activator (B) and inhibitor (D) activity.

### DETAILED DESCRIPTION OF THE INVENTION

A method of spatiotemporally controlling G protein cell signaling has been developed. Using a method of the invention, it is now possible to control specific cell signaling pathways within a selected region of a cell in a spatial and temporal manner. Advantageously, a method of the invention allows precise control of cell behavior not possible using diffusible molecules previously used to control cell signaling. By spatiotemporally controlling cell signaling pathways, methods of the invention also provide means for spatiotemporally controlling cell behavior.

### I. Method of Modulating Localized G Protein Signaling

In an aspect, the present invention provides methods of modulating G protein signaling in a localized region of a cell using an artificial optical input. Generally, the method comprises (a) introducing at least one exogenous opsin into a cell, wherein the exogenous opsin comprises a light sensing domain of a melanopsin or a metazoan color opsin and a G protein coupled receptor (GPCR) activation domain that affects G protein signaling; and (b) changing an artificial optical input in a localized region on and/or adjacent to the cell's surface, wherein the activation state of the exogenous opsin within the localized region is affected when the light sensing domain detects a change in the artificial optical input, thereby resulting in the GPCR activation domain modulating G protein signaling.

In another aspect, the present invention provides methods for modulating a cell behavior that is controlled by localized G protein signaling in the cell. The method comprises modulating G protein signaling in a localized region of a cell using an artificial optical input. Generally, the method comprises (a) introducing at least one exogenous opsin into a cell, wherein the exogenous opsin comprises a light sensing domain of a melanopsin or a metazoan color opsin and a G protein coupled receptor (GPCR) activation domain that affects G protein signaling; and (b) changing an artificial optical input in a localized region on and/or adjacent to the cell's surface, wherein the activation state of the exogenous opsin within the localized region is affected when the light sensing domain detects a change in the artificial optical input, thereby resulting in the GPCR activation domain modulating G protein signaling.

As used herein, the term “optical activation” refers to localized G protein signaling induced by a method of the invention.

As used herein, the phrases “G protein signaling in a localized region of a cell” and “localized G protein signaling” are used interchangeably and refer to spatially controlled G protein signaling. Localized G protein signaling requires differential spatial activation of GPCRs on a cell's surface. In the present invention, this is achieved by spatial confinement of an artificial optical input. Stated another way, localized G protein signaling is a result of selective activation of a subset of GPCRs on a cell's surface and is not the result of global activation of all GPCRs. Thus, localized G protein signaling may also be described as asymmetrical G protein signaling. As used herein, the terms “localized G protein signaling” and “asymmetrical G protein signaling” are used interchangeably. Modulating localized G protein signaling may either increase or decrease G protein signaling.

Theoretically, an optical input can manipulate G protein signaling in all regions on the surface of a cell by activating an exogenous opsin on the cell's surface. According to the invention, though, at any one time activation is limited to a confined (i.e. localized) region on the cell's surface. For instance, a localized region may be about 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95 or about 98% of a cell's surface area. In some embodiments, a localized region may be about 0.10, about 0.15, about 0.20, about 0.25, about 0.30, about 0.35, about 0.40, about 0.45, about 0.50, about 0.55, about 0.60, about 0.65, about 0.70, about 0.75, about 0.80, about 0.85, about 0.90, about 0.95, or about 1% of a cell's surface area. In other embodiments, a localized region may be about 0.50, about 0.55, about 0.60, about 0.65, about 0.70, about 0.75, about 0.80, about 0.85, about 0.90, about 0.95, about 1, about 2, about 3, about 4, about 5, about 6, about 7, about 8, about 9, about 10, about 11, about 12, about 13, about 14, or about 15% of a cell's surface area. In yet other embodiments, a localized region may be about 10, about 11, about 12, about 13, about 14, about 15, about 16, about 17, about 18, about 19, about 20, about 21, about 22, about 23, about 24, or about 25% of a cell's surface area. In yet other embodiments, a confined region may be about 20, about 21, about 22, about 23, about 24, about 25, about 26, about 27, about 28, about 29, about 30, about 31, about 32, about 33, about 34, or about 35% of a cell's surface area. In other embodiments, a confined region may be about 30, about 31, about 32, about 33, about 34, about 35, about 36, about 37, about 38, about 39, about 40, or about 45% of a cell's surface area. In other embodiments, a confined region may be about 40, about 45, about 50, about 55, about 60, about 65, about 70, about 75, about 80, about 85, about 90, or about 95% of a cell's surface area. In some preferred embodiments, a confined region may be about 0.25, about 0.30, about 0.35, about 0.40, about 0.45, about 0.50, about 0.55, about 0.60, about 0.65, about 0.70, or about 0.75% of a cell's surface area. In other preferred embodiments, a localized region may be about 0.25% to about 1.0% of a cell's surface area. In other preferred embodiments, a localized region may be about 0.25% to about 0.75% of a cell's surface area. In other preferred embodiments, a localized region may be about 0.25% to about 0.50% of a cell's surface area. In other preferred embodiments, a localized region may be about 0.10% to about 0.50% of a cell's surface area. In other preferred embodiments, a localized region may be about 0.15% to about 0.50% of a cell's surface area. In other preferred embodiments, a localized region may be about 0.20% to about 0.50% of a cell's surface area. In an exemplary embodiment, a localized region may be about 0.45% of a cell's surface area. In other preferred embodiments, a localized region may be about 0.45% to about 0.55% of a cell's surface area. In another exemplary embodiment, a localized region may be about 0.50% of a cell's surface area. In yet another exemplary embodiment, a localized region may be about 0.55% of a cell's surface area.

As used herein, the term “optical input” refers to photons (light) illuminating a cell. An “artificial optical input” refers to photons produced from an artificial light source (i.e. not a natural light). For example, sunlight is a natural light and, therefore, is not used to produce an artificial optical input according the invention. To reiterate a point made above, a critical aspect of the invention is the differential spatial activation of at least one GPCR, such as an opsin, on a cell's surface. This is achieved by spatial confinement of an artificial optical input, wherein the artificial optical input is created by controlling an optical signal (e.g. a pulse of light) directed to or adjacent to the cell's surface. Advantageously, the optical signal itself does not need to directly touch the cell surface, provided the edge of the cell is still activated. As detailed in the Examples, the continuous relocation of an optical signal away from a macrophage induces asymmetrical G protein signaling and a migratory response in the macrophage. Other aspects of an artificial optical input of the invention are detailed below.

In another aspect, the present invention provides a method of spatiotemporally controlling cell behavior by introducing an exogenous opsin into a cell and spatiotemporally activating the opsin in desired confined regions of the cell by creating an optical input adjacent to the cell.

Other aspects of the method are described in further detail below.

(A) exogenous Opsin

As used herein, the term “exogenous opsin” refers to an opsin that is not typically present in a particular cell type. In mammalian cells, for example, rhodopsins are found in rod photoreceptor cells, color opsins are found in cone photoreceptor cells, and melanopsins are found in photosensitive ganglion cells of the retina. Rhodopsins, color opsins and melanopsins are not typically present in other cell types including, but not limited to, cardiac cells, neural cells, blood cells, skin cells, certain tumor cells, and immune cells.

Opsins are a large family of light-sensitive membrane-bound G protein coupled receptors (GPCRs). Opsins are involved in visual and non-visual responses, mediating the conversion of a photon of light into an electrochemical signal, the first step in the visual transduction cascade. Light absorption induces changes in the molecular structure of an opsin that allows it to activate a G protein. The G protein mediates an enzymatic signaling cascade that eventually generates an electrical response in the photoreceptor cell. The signal received from an opsin is amplified at this stage since one opsin molecule can activate many G proteins. Different opsin families are coupled to specific types of G proteins that produce different responses. The downstream signaling cascade depends on the G protein subtype, because different G proteins can act through different pathways. G protein subtypes are typically defined by the Gα subunit, for example, Gαi/o subunit, Gαq subunit, Gαs subunit, Gal 2 subunit, Ga transducin-rod and Gα transducing-cone. G proteins are described in further detail below.

It is known in the art that GPCRs, including opsins, have both an active state and an inactive state. As used herein, the phrase “activation state of an exogenous opsin” refers to both the active state (active conformation) and the inactive state (inactive conformation). A change in the activation state refers to either a change from the inactive state to the active state, or to the inactive state from an active state.

The general structure of opsins are conserved, with a light-sensing domain comprising a chromophore-binding region and an activation domain capable of activating a G protein by binding to and inducing the exchange of GDP for a GTP. As used herein, the term “light sensing domain” refers to extracellular and transmembrane regions of an opsin that interact with and/or are critical for retinal binding (retinal is a photoreactive chromophore necessary for opsin function) and comprise the amino acid residues that confer the receptor's light-sensing activity and photoactivity. As used herein, the term “activation domain” refers to intracellular regions of an opsin that interact with a G protein. Methods for identifying a light sensing domain and an activation domain are known in the art. Crystal structures have been published for both opsin and rhodopsin in active conformations, with or without the binding of a peptide derived from the C-terminal helix α5 of the α subunit of G protein transducin (PNAS 2006 103:16123-8; Nature 2011 471:656-60; Nature 2008 454:183-7; Nature 2008:455:497-502; Nature 2011 471:651-5). These crystal structures, in combination with biochemical and biophysical studies, have led to a common understanding of the general structure of opsins. For a review, see Acta Pharmacol Sin 2012 33(03):291-299. Sequence alignments, preferably at the amino acid level, may be made between one or more opsins for which light sensing and activation domains have been defined and one or more opsins for which light sensing and activation domains have not been defined in order to identify light sensing and activation domains in these opsins. Further details may be found in the Examples, as well as in Biochemistry 2005 44(7): 2284-2292. The references described in this paragraph are each herein incorporated by reference it their entirety.

Only a subset of opsins are suitable for the present invention. Specifically, suitable opsins diffuse slowly along the plasma membrane and deactivate rapidly in the absence of a signal. These properties, in combination with an artificial optical input confined to a limited region of a cell's surface, allows for spatial control of G protein activation. The latter also allows for temporal control of G protein activation. Additionally, a suitable opsin will have spectral selectivity. As used herein, the term “spectrally selective” means that an opsin activated by one or more specific wavelengths of light. For instance, in some embodiments, a specific wavelength used to globally image cellular and molecular responses using fluorescent protein reporters does not interfere with the localized activation of the opsin. For example, in some applications it is desirable to image a cell's response dynamics without activating an opsin of the invention. To do so requires the use of a spectrally selective opsin that is not activated by the wavelength used for imaging. Additional advantages of spectrally selective opsin are described in further detail below, and will also be apparent to one skilled in the art. While most opsins may diffuse slowly, only a subset deactivate rapidly.

In some embodiments, an opsin of the invention is a melanopsin. In other embodiments, an opsin of the invention is a metazoan G-opsin responsible for color vision (a “metazoan color opsin). Non limiting examples of suitable metazoan color include jellyfish opsin, mammalian long wavelength sensitive opsin (red opsin), mammalian middle wavelength sensitive opsin (green opsin), and short wavelength sensitive opsin (blue opsin). In some embodiments, an opsin is a mammalian red opsin. In other embodiments, an opsin is a mammalian green opsin. In still other embodiments, an opsin is a mammalian blue opsin. In different embodiments, an opsin is a jellyfish opsin. In some preferred embodiments, an opsin is a human red opsin. In other preferred embodiments, an opsin is a human blue opsin. In still other preferred embodiments, an opsin is a human green opsin. In an exemplary embodiment, a jellyfish opsin comprises SEQ ID NO: 1. In another exemplary embodiment, a jellyfish opsin consists of SEQ ID NO: 1. In another exemplary embodiment, a mammalian blue opsin comprises SEQ ID NO: 2. In another exemplary embodiment, a mammalian blue opsin consists of SEQ ID NO: 2. In another exemplary embodiment, a mammalian blue opsin comprises SEQ ID NO: 2. In another exemplary embodiment, a mammalian blue opsin is a homolog of SEQ ID NO: 2. In another exemplary embodiment, a mammalian red opsin comprises SEQ ID NO: 4. In another exemplary embodiment, a mammalian red opsin consists of SEQ ID NO: 4. In another exemplary embodiment, a mammalian red opsin is a homolog of SEQ ID NO: 4. In another exemplary embodiment, a mammalian green opsin comprises SEQ ID NO: 5. In another exemplary embodiment, a mammalian green opsin consists of SEQ ID NO: 5. In another exemplary embodiment, a mammalian green opsin is a homolog of SEQ ID NO: 5. In another exemplary embodiment, a mammalian melanopsin comprises SEQ ID NO: 6. In another exemplary embodiment, a mammalian melanopsin consists of SEQ ID NO: 6. In another exemplary embodiment, a mammalian melanopsin is a homolog of SEQ ID NO: 6.

In other embodiments, an opsin of the invention is a chimeric. As used herein, the term “chimeric opsin” refers to a recombinant protein comprising a light sensing domain from a first opsin and an activation domain from a second GPCR. The second GPCR may or may not be an opsin. Advantageously, independent selection of a light sensing domain and a GPCR activation domain generates an opsin that is uniquely designed to be spectrally tuned to a specific wavelength (specificity provided by the light sensing domain) and activate a specific G protein subtype (specificity provided by the activation domain). This approach allows the use of any wavelength to activate any downstream signaling cascade in order to steer cellular behavior. In some embodiments, an opsin of the invention is a chimeric opsin comprising a light sensing domain of a metazoan color opsin. In other embodiments, an opsin of the invention is a chimeric opsin comprising a light sensing domain of a mammalian blue opsin. In still other embodiments, an opsin of the invention is a chimeric opsin comprising a light sensing domain of a mammalian red opsin. In yet other embodiments, an opsin of the invention is a chimeric opsin comprising a light sensing domain of a mammalian green opsin. In additional embodiments, an opsin of the invention is a chimeric opsin comprising a light sensing domain of a melanopsin. In different embodiments, an opsin of the invention is a chimeric opsin comprising an activating domain of an opsin that activates a Gαs subunit. In still different embodiments, an opsin of the invention is a chimeric opsin comprising an activating domain of an opsin that activates a Gαi/o subunits. In yet different embodiments, an opsin of the invention is a chimeric opsin comprising an activating domain of an opsin that activates a Gαq subunit. In alternative embodiments, an opsin of the invention is a chimeric opsin comprising an activating domain of an opsin that activates a Gα12/13 subunit. In other embodiments, an opsin of the invention is a chimeric opsin comprising an activating domain of an opsin that activates transducin. In further embodiments, an opsin of the invention is a chimeric opsin listed in Table A. In some preferred embodiments, an opsin of the invention is a chimeric opsin comprising a light sensing domain from a human color opsin selected from the group consisting of a blue opsin, a green opsin and a red opsin. In other preferred embodiments, an opsin of the invention is a chimeric opsin comprising a light sensing domain of a mammalian blue opsin and an intracellular activating domain of a jellyfish opsin that activates a mammalian Gαs subunit. In an exemplary embodiment, an opsin of the invention is a chimeric opsin comprising SEQ ID NO: 3. In yet another exemplary embodiment, an opsin of the invention is a chimeric opsin consisting of SEQ ID NO: 3.

TABLE A

| | Light Sensing Domain of an Opsin | | | | Intracellular Activating Domain of an Opsin | | |
|---|---|---|---|---|---|---|---|
| | Red | Blue | Green | | Jellyfish | | |
| | Opsin | Opsin | Opsin | Melanopsin | Opsin | Rhodopsin | Melanopsin |
| Chimeric 1 | X | | | | X | | |
| Chimeric 2 | X | | | | | X | |
| Chimeric 3 | X | | | | | | X |
| Chimeric 4 | | X | | | X | | |
| Chimeric 5 | | X | | | | X | |
| Chimeric 6 | | X | | | | | X |
| Chimeric 7 | | | X | | X | | |
| Chimeric 8 | | | X | | | X | |
| Chimeric 9 | | | X | | | | X |
| Chimeric 10 | | | | X | X | | |
| Chimeric 11 | | | | X | | X | |
| Chimeric 12 | | | | X | | | X |

It is generally known in the art which Gα subunit a known opsin activates. See for example Prog Retin Eye Res. 2001 January; 20(1):49-94 (PMID: 11070368); Philos Trans R Soc Lond B Biol Sci. 2009 Oct. 12; 364(1531):2881-95 (PMID: 19720651), incorporated herein by reference in its entirety. Methods for identifying melanopsins and metazoan color opsins are also known in the art. See for example J Anim Physiol Anim Nutr 2012 Nov. 22 (PMID 23173557), incorporated herein by reference in its entirety. Further, nucleotide and amino acid sequences of many melanopsins and metazoan color opsins are deposited in NCBI. The NCBI Accession numbers for blue (short-wave-sensitive) opsins from several species are as follows: AAL31362.1 and NP_{—}001699 (human), Gene ID: 101080701 (Felis catus), Gene ID: 12057 (Mus musculus), Gene ID: 482267 (Canis lupus familaris), Gene ID: 100071755 (Equus caballus), Gene ID: 81644 (Rattus norvegicus), Gene ID: 100353418 (Oryctolagus cuniculus). The NCBI Accession numbers for green (medium-wave-sensitive) opsins from several species are as follows: Gene ID: Gene ID: 2652, Gene ID: 728458, NP_{—}000504 (human), Gene ID: 14539 (Mus musculus), Gene ID: 89810 (Rattus norvegicus), Gene ID: 100008674 (Oryctolagus cuniculus). The NCBI Accession numbers for red (long-wave-sensitive) opsins from several species are as follows: Gene ID: 5956, NP_{—}064445.2 (human), Gene ID: 493959 (Felis catus), Gene ID: 20164 (Mus musculus), Gene ID: 403778 (Canis lupus familaris), Gene ID: 100033892 (Equus caballus). Amino acid sequences can be determined from nucleic acid sequences using methods known in the art. Homologs can be found in other species by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details. Generally a homolog will have a least 80, 81, 82, 83, 84, 85, 86, 87, 88, or 89% homology. In another embodiment, a homolog may have at least 90, 91, 92, 93, 94, 95, 96, 97, 98, 99, or 100% homology.

All the nucleic acid and amino acid sequences of the invention may be obtained using a variety of different techniques known in the art. Nucleotide and amino acid sequences may be isolated or produced using standard techniques, purchased or obtained from a depository. Once a nucleotide sequence is obtained, it may be amplified for use in a variety of applications, using methods known in the art. Methods of making recombinant proteins are also well known in the art. Additional information may be found in Sambrook et al., Molecular Cloning: A Laboratory Manual (New York: Cold Spring Harbor Laboratory Press, 1989), incorporated herein by reference.

(B) G proteins

According to the invention, optical activation of an opsin controls cell behavior by activating a G protein. As such, optical activation of an opsin may be measured by measuring G protein activation in a cell.

G proteins may be heterotrimeric G proteins, or monomeric. In preferred embodiments, G proteins of the invention are heterotrimeric G proteins. Heterotrimeric G proteins may comprise α, β, and γ subunits. When an inactive Gαβγ heterotrimeric G proteins is activated by a GPCR, such as an opsin, the G protein exchanges GDP for GTP, which allows it to dissociate into two molecules: a GTP-bound α subunit and a βγ complex. Separation of the subunits exposes the active site of Gα, allowing it to act on its effector enzyme. Active Gα subunits activate different signaling cascades (or second messenger pathways) and effector proteins, while the receptor is able to activate the next G protein. Gα has an intrinsic GTPase activity and the subunits remain active until Gα hydrolyses bound GTP to GDP. GDP-bound Gα binds to Gβγ once again and together they hide their active sites, effectively suppressing their activity. Gα subunits include Gαs (G stimulatory), Gαi (G inhibitory), Gαo (G other), Gαq, and Gα12/13 subunits.

Methods of detecting G protein activation in a cell are known in the art. For instance, activation of G protein by an opsin may be detected by measuring any downstream effect of G protein activation. Non-limiting examples of downstream effects of G protein activation may include Gβγ translocation, a change in cAMP levels, establishment of a PIP3 concentration gradient, cytoskeleton remodeling (including remodeling of actin), cell migration (including translocation of a macrophage cell and other immune cells), neurite outgrowth or extension in a differentiating neuron. Alternatively, activation of a G protein may be detected by measuring the interaction between a GPCR and one or more G protein subunits; or between one or more G protein subunits and effector proteins. Any method capable of detecting protein-protein interaction may be used to measure activation of a G protein. Measurements may be qualitative, semi-quantitative or quantitative. Non limiting examples of methods of measuring protein-protein interactions that may be used to detect and/or quantify activation of G proteins include fluorescent resonance energy transfer (FRET), lanthanide resonance energy transfer (LRET), fluorescence cross-correlation spectroscopy, fluorescence quenching, fluorescence polarization, flow cytometry, scintillation proximity, luminescence resonance energy transfer, direct quenching, ground-state complex formation, chemiluminescence energy transfer, bioluminescence resonance energy transfer, excimer formation, colorimetric substrates detection, phosphorescence, electrochemical changes, and redox potential changes.

In some embodiments, G protein activation may be detected and/or quantified by measuring Gβγ translocation. Both forward and reverse translocation rates may be measured, providing a measurement of G protein activation and deactivation, respectively. For instance, a Gβγ subunit may be tagged using a fluorescent reporter to monitor Gβγ translocation. In some embodiments, a Gγ subunit is tagged. Non-limiting examples of Gγ subunits include γ9, γ5, and γ3. In other embodiments, a Gβ subunit is tagged. In an exemplary embodiment, Gγ9 is tagged using a fluorescent protein to monitor Gγ translocation. Further details may be found in the Examples, or in PNAS 109(51):E3568-77 and Biochem Biophys Res Commun 2012 421(3): 605-11, each hereby incorporated by reference in its entirety.

G proteins relay environmental signals external to the cell received from GPCRs, such as opsins, to modulate a variety of cell behaviors and physiological responses. As such, methods of measuring G protein activation by an opsin in a cell may also include measuring any cell behaviors and physiological responses relayed by G protein activation. Downstream effects of G protein activation may include regulating metabolic enzymes, ion channels, transporters, and other parts of the cell machinery, controlling transcription, motility, contractility, and secretion, which in turn regulate systemic functions such as embryonic development, learning and memory, and homeostasis. Differential spatial activation of GPCRs across a cell mediates migration in numerous cell types, including, but not limited to, of immune cells, invasive cancer cells and cells undergoing morphogenesis. Differential spatial activation of GPCRs in a neuron induces neurite outgrowth. In some embodiments, G protein activation may be detected and/or quantified by measuring a change in cAMP levels in a cell. In other embodiments, G protein activation may be detected and/or quantified by measuring PIP3 levels in a cell. In yet other embodiments, G protein activation may be detected and/or quantified by measuring a PIP3 concentration gradient, including the establishment of a PIP3 gradient, the reversal of a PIP3 gradient, and/or the dissipation of a PIP gradient. In still other embodiments, G protein activation may be detected and/or quantified by measuring a model parameter in Table 4. In some embodiments, G protein activation may be detected and/or quantified by measuring remodeling of actin. In different embodiments, G protein activation may be detected and/or quantified by measuring cell migration. In still different embodiments, G protein activation may be detected and/or quantified by measuring neurite growth. In further embodiments, G protein activation may be detected and/or quantified by measuring lamellipodia formation. In other embodiments, G protein activation may be detected and/or quantified by measuring the interaction between GPCR and a G protein subunit. In yet other embodiments, G protein activation may be detected and/or quantified by measuring the interaction between one or more G protein subunits and one or more effector proteins. Such methods are well known in art and further detailed in the Examples.

Methods of measuring G protein activation in a cell may comprise using a fluorescent reporter. As used herein, the term “fluorescent reporter” may be used to describe any reporter that may typically result in fluorescence or luminescence of the cell. For instance, a fluorescent protein may be used, such as Y66H, Y66F, EBFP, EBFP2, Azurite, GFPuv, T-Sapphire, Cerulean, mCFP, ECFP, CyPet, Y66W, mKeima-Red, TagCFP, AmCyan1, mTFP1, S65A, Midoriishi Cyan, Wild Type GFP, S65C, TurboGFP, TagGFP, S65L, Emerald, S65T, EGFP, Azami Green, ZsGreen1, TagYFP, EYFP, Topaz, Venus, mCitrine, YPet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoerythrin (BPE), mCherry, HcRed1, Katusha, P3, Peridinin Chlorophyll (PerCP), mKate (TagFP635), TurboFP635, mPlum, mRaspberry or other suitable fluorescent protein. Additionally, a photoprotein capable of bioluminescence, such as a luciferase, or a fluorescent dye may be used. Non limiting examples of fluorescent dyes that may be used to detect G protein activation may include xanthene dye derivatives such as fluorescein, rhodamine, Oregon green, eosin, and Texas red, cyanine dye derivatives such as cyanine, indocarbocyanine, oxacarbocyanine, thiacarbocyanine, and merocyanine, naphthalene dye derivatives, coumarin dye derivatives, oxadiazole dye derivatives such as pyridyloxazole, nitrobenzoxadiazole and benzoxadiazole, pyrene dye derivatives such as cascade blue, oxazine dye derivatives such as Nile red, Nile blue, cresyl violet, and oxazine 170, acridine dye derivatives such as proflavin, acridine orange, and acridine yellow, arylmethine dye derivatives such as auramine, crystal violet, and malachite green, and tetrapyrrole dye derivatives such as porphin, phtalocyanine, and bilirubin. Fluorescence detection may occur by any method known in the art.

When a fluorescent reporter is used to measure G protein activation by an opsin, any combination of fluorescent reporter and opsin may be used, provided the wavelength used to excite the fluorescent reporter, or the emission wavelength of the fluorescent reporter and the wavelength capable of activating the opsin do not overlap. Overlap of the wavelength used to excite the fluorescent reporter, or the emission wavelength of the fluorescent reporter with the wavelength capable of activating the opsin may activate the opsin globally, therefore preventing spatially confined activation of the opsin. In an exemplary embodiment, a G protein may be activated by a blue opsin and G protein activation may be measured using a green fluorescent protein. In another exemplary embodiment, a G protein may be activated by a blue opsin and G protein activation may be measured using mCherry.

### (C) Cells

Applicants have discovered that an exogenous opsin introduced into a cell that typically does not comprise the exogenous opsin, will activate G proteins in the cell. According to the invention, an exogenous opsin may be introduced into any cell. In some embodiments, an exogenous opsin may be introduced in vitro into a cell from a cell line. In some alternatives of the embodiments, a cell line may be a primary cell line (i.e. derived from a primary culture of cells isolated from a subject). Methods of preparing a primary cell line utilize standard techniques known to individuals skilled in the art. In other alternatives, a cell line may be an established cell line. A cell line may be adherent or non-adherent, or a cell line may be grown under conditions that encourage adherent, non-adherent or organotypic growth using standard techniques known to individuals skilled in the art.

In some embodiments, a cell line may be an established human cell line derived from a tumor. Non-limiting examples of cell lines derived from a tumor may include the osteosarcoma cell lines 143B, CAL-72, G-292, HOS, KHOS, MG-63, Saos-2, and U-20S; the prostate cancer cell lines DU145, PC3 and Lncap; the breast cancer cell lines MCF-7, MDA-MB-438 and T47D; the myeloid leukemia cell line THP-1, the glioblastoma cell line U87; the neuroblastoma cell line SHSY5Y; the bone cancer cell line Saos-2; the colon cancer cell lines WiDr, COLO 320DM, HT29, DLD-1, COLO 205, COLO 201, HCT-15, SW620, LoVo, SW403, SW403, SW1116, SW1463, SW837, SW948, SW1417, GPC-16, HCT-8HCT 116, NCI-H716, NCI-H747, NCI-H508, NCI-H498, COLO 320HSR, SNU-C2A, LS 180, LS 174T, MOLT-4, LS513, LS1034, LS411N, Hs 675.T, CO 88BV59-1, Co88BV59H21-2, Co88BV59H21-2V67-66, 1116-NS-19-9, TA 99, AS 33, TS 106, Caco-2, HT-29, SK-CO-1, SNU-C2B and SW480; HeLa, CHO, NIT1, HL-1, HL-60, Raw 264.7, Jurkat, and the pancreatic carcinoma cell line Panc1.

In other embodiments, a cell line may be an established cell line routinely used in the lab, such as a HeLa cell line.

In other embodiments, a cell may be an immune cell. For example, a cell may be a macrophage cell, a T-cell, a B-cell, a monocyte, a neutrophil, an eosinophil, and a dendritic cell. In a preferred embodiment, an immune cell is a macrophage. In another preferred embodiment, an immune cell is a dendritic cell. In another preferred embodiment, an immune cell is a T-cell. In another preferred embodiment, an immune cell is a neutrophil. In another preferred embodiment, an immune cell is a monocyte. Non-limiting examples of immune cells that may be used in the invention may include a human macrophage cell, a human neutrophil cell, a human dendritic cell, a human T-cell, a mouse macrophage from a cell line such as RAW 264.7 cell line, primary cells obtained from peritoneal and lung lavage, primary neutrophils, Schwann cells, and astrocytes. In some preferred embodiments, a cell is a mouse macrophage from a RAW 264.7 cell line.

In other preferred embodiments, a cell may be a neuron cell. In a preferred embodiment, a cell may be a primary neuron cell. Non-limiting examples of neuron cells that may be used in the invention may include a human primary neuron cell, and a rat neuron cell such as a post natal 1-2 day old hippocampal neuron cell. In some preferred embodiments, a cell is a post natal 1-2 day old primary neuron cell.

### (D) Changing an Artificial Optical Input in a Localized Region on a Cell's Surface

Spatial confinement of an artificial optical input limits activation of the exogenous opsins on a cell's surface to only those opsins within a defined region. In some embodiments, optical input is defined as the area of illumination. An artificial optical input may be about 0.25, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 125, 130, 135, 140, 145, or about 150 μm² or more. In some embodiments, an optical input area is about 0.25, 0.3, 0.4, 0.5, 0.6, 0.7, 0.8, 0.9, or about 1 μm². In other embodiments, an optical input area is about 1, 2, 3, 4, 5, 6, 7, 8, 9, 10, 11, 12, 13, 14, or about 15 μm². In yet other embodiments, an optical input area is about 10, 11, 12, 13, 14, 15, 16, 17, 18, 19, 20, 21, 22, 23, 24, or about 25 μm². In other embodiments, an optical input area is about 20, 21, 22, 23, 24, 25, 26, 27, 28, 29, 30, 31, 32, 33, 34, or about 35 μm². In still other embodiments, an optical input area is about 30, 31, 32, 33, 34, 35, 36, 37, 38, 39, or about 40 μm². In other embodiments, an optical input area is about 40, 45, 50, 55, 60, 65, 70, or about 75 μm². In additional embodiments, an optical input area is about 70, 75, 80, 85, 90, 95, 100, or about 105 μm². In other embodiments, an optical input area is about 100, 105, 110, 115, 120, 125, 130, or about 135 μm². In yet other embodiments, an optical input area is about 130, 135, 140, 145, or about 150 μm² or more. In an exemplary embodiment, an optical input area is about 3, about 4, or about 5 μm². In another exemplary embodiment, an optical input area is about 1 μm². In yet another exemplary embodiment, the optical input area is about 0.5 μm².

An artificial optical input in a localized region on a cell's surface may be created by directing spatially controlled pulses of light (i.e. an optical signal) to or adjacent to a cell's surface. Methods of generating spatially controlled pulses of light are known in the art. For instance, pulses of light may be spatially controlled by the use of filters to control the area illuminated, or the use of a laser light source. In preferred embodiments, an optical input may be spatially controlled by using a laser light source. The wavelength of light used is dependent on the light sensing domain of the exogenous opsin. Each opsin reaches peak light absorption at a known wavelength. Other wavelengths of light around that wavelength will activate the signaling system with decreased efficiency. Peak absorption for melanopsin, blue opsin, green opsin and red opsin are about 488 nm, about 414 to about 420 nm, about 530 to about 540 nm, and about 560 nm, respectively. It is well known in the art that certain amino acid residues, termed spectral tuning sites, have a strong effect on λmax values. A skilled artisan will appreciate that it is possible to selectively mutate these residues using site-directed mutagenesis to change the light absorption properties of an opsin. The impact of spectral tuning sites on λmax differs between different opsin groups and between opsin groups of different species. For a comprehensive review of spectral tuning sites see Prog Retin Eye Res (2000) 19(4): 385-419 and Clin Genet (2005) 67(5): 369-77.

An optical signal, and therefore the optical input created, may be adjusted or changed to manipulate G protein signaling in a localized region on the surface of a cell expressing an exogenous opsin. In some embodiments, an artificial optical input may be changed to increase G protein signaling. In other embodiments, an artificial optical input may be changed to decrease G protein signaling. In still other embodiments, an artificial optical input may be changed to initiate G protein signaling. In yet other embodiments, an artificial optical input may be changed to stop (or terminate) G protein signaling. In different embodiments, an artificial optical input may be changed to relocate G protein signaling to a different region of a cell. In alternative embodiments, an artificial optical input may be changed to control the magnitude of signaling activities in a cell expressing an opsin. As used herein, the term “magnitude of signaling activities” may be used to describe the level of signaling generated by an optical input and therefore the level of modulation of cell behavior and physiological responses. For instance, if an artificial optical input is used to initiate and direct immune cell migration in an immune cell expressing an exogenous opsin, the magnitude of immune cell migration (e.g. distance, velocity or duration) may be controlled by adjusting the artificial optical input. Similarly, if an artificial optical input is used to initiate neurite outgrowth in a neuron expressing an exogenous opsin, the magnitude of neurite outgrowth (e.g. rate of neurite formation, etc.) may be controlled by adjusting the artificial optical input. Also, if an artificial optical input is used to control contractility in a cardiomyocyte expressing an exogenous opsin, the magnitude of contraction (e.g. rate of contraction/beating, duration, etc.) may be controlled by adjusting the artificial optical input. Other iterations are also contemplated as supported by the disclosures herein, and will be apparent to a skilled artisan. As used herein, the terms “adjusted” and “changed” are used interchangeably.

An artificial optical input may be adjusted by altering aspects of the optical signal creating the artificial optical input. Non-limiting examples of aspects of an optical signal that may be altered in order to change an artificial optical input include the number of light pulses, the frequency of light pulses, the intensity of light pulses, the duration (dwell time) of each light pulse, the distance of the light pulses from the cell periphery, the size of the artificial optical input, or combinations thereof. The method of adjusting an artificial optical input can and will vary depending on the cell type, the size of the cell, the opsin, the intensity of the light source, and the cell behavior being spatially controlled, and may be determined with routine experimentation.

In some embodiments, an artificial optical input may be adjusted by controlling the number of light pulses created. For instance, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or more light pulses may be created to adjust an artificial optical input. In some embodiments, about 1, 2, 3, 4, 5, 6, 7, 8, 9, 10, 11, 12, 13, 14, or about 15 light pulses are created to adjust an artificial optical input. In other embodiments, about 10, 11, 12, 13, 14, 15, 16, 17, 18, 19, 20, 21, 22, 23, 24, or about 25 light pulses are created to adjust an artificial optical input. In yet other embodiments, about 20, 21, 22, 23, 24, 25, 26, 27, 28, 29, 30, 31, 32, 33, 34, or about 35 light pulses are created to adjust an artificial optical input. In other embodiments, about 30, 31, 32, 33, 34, 35, 36, 37, 38, 39, or about 40 light pulses are created to adjust an artificial optical input. In still other embodiments, about 40, 45, 50, 55, 60, 65, 70, 75, 80, 85, 90, 95, or more light pulses are created to adjust an artificial optical input. In a preferred embodiment, about 4, 5, 6, 7, 8, 9, 10, or 11 light pulses are created to adjust an artificial optical input. In an exemplary embodiment, one light pulse is created to adjust an artificial optical input. In an exemplary embodiment, 5 light pulses are created to adjust an artificial optical input. In yet another exemplary embodiment, 10 light pulses are created to adjust an artificial optical input. In another exemplary embodiment, 25 light pulses are created to adjust an artificial optical input.

In other embodiments, an artificial optical input may be adjusted by controlling the frequency of light pulses created. The frequency of light pulses refers to the number of pulses per unit of time. In some embodiments, pulse frequency may increase. In other embodiments, pulse frequency may decrease. In alternative embodiments, a light pulse may be stopped. Pulse frequency may range from a few pulses per second to about 1 pulse per minute. For example, a suitable pulse frequency may be about 2 pulses/second, about 3 pulses/second, about 4 pulses/second, about 5 pulses/second, about 6 pulses/second, about 7 pulses/second, about 8 pulses/second, about 9 pulses/second, or about 10 pulses/second. Alternatively, a suitable pulse frequency may be about 1 pulse/second, about 1 pulse/2 seconds, about 1 pulse/3 seconds, about 1 pulse/4 seconds, about 1 pulse/5 seconds, about 1 pulse/6 seconds, about 1 pulse/7 seconds, about 1 pulse/8 seconds, about 1 pulse/9 seconds, about 1 pulse/10 seconds, about 1 pulse/11 seconds, about 1 pulse/12 seconds, about 1 pulse/13 seconds, about 1 pulse/14 seconds, about 1 pulse/15 seconds, about 1 pulse/16 seconds, about 1 pulse/17 seconds, about 1 pulse/18 seconds, about 1 pulse/19 seconds, about 1 pulse/20 seconds, about 1 pulse/21 seconds, about 1 pulse/22 seconds, about 1 pulse/23 seconds, about 1 pulse/24 seconds, about 1 pulse/25 seconds, about 1 pulse/26 seconds, about 1 pulse/27 seconds, about 1 pulse/28 seconds, about 1 pulse/29 seconds, about 1 pulse/30 seconds, about 1 pulse/31 seconds, about 1 pulse/32 seconds, about 1 pulse/33 seconds, about 1 pulse/34 seconds, about 1 pulse/35 seconds, about 1 pulse/36 seconds, about 1 pulse/37 seconds, about 1 pulse/38 seconds, about 1 pulse/39 seconds, about 1 pulse/40 seconds, about 1 pulse/41 seconds, about 1 pulse/42 seconds, about 1 pulse/43 seconds, about 1 pulse/44 seconds, about 1 pulse/45 seconds, about 1 pulse/46 seconds, about 1 pulse/47 seconds, about 1 pulse/48 seconds, about 1 pulse/49 seconds, about 1 pulse/50 seconds, about 1 pulse/51 seconds, about 1 pulse/52 seconds, about 1 pulse/53 seconds, about 1 pulse/54 seconds, about 1 pulse/55 seconds, about 1 pulse/56 seconds, about 1 pulse/57 seconds, about 1 pulse/58 seconds, about 1 pulse/59 seconds, or about 1 pulse/60 seconds. In some preferred embodiments, a suitable pulse frequency is about 5 pulses/second to about 1 pulse/30 seconds. In other preferred embodiments, a suitable pulse frequency is about 5 pulses/second to about 1 pulse/15 seconds. In still other preferred embodiments, a suitable pulse frequency is about 3 pulses/second to about 1 pulse/15 seconds.

In other embodiments, an artificial optical input may be adjusted by controlling the intensity of light pulses created. The intensity of the light pulses may be about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 or more μW. In some embodiments, the intensity of the light pulses is about 0.1, 0.15, 0.2, 0.25, 0.3, 0.35, 0.4, 0.45, or about 0.5 μW. In other embodiments, the intensity of the light pulses is about 0.5, 0.55, 0.6, 0.65, 0.7, 0.75, 0.8, 0.85, 0.9, 0.95, or about 1 μW. In yet other embodiments, the intensity of the light pulses is about 1, 1.5, 2, 2.5, 3, 3.5, 4, 4.5, 5, 5.5, 6, 6.5, 7, 7.5, 8, 8.5, 9, 9.5, or about 10 μW. In some embodiments, the intensity of the light pulses is about 10, 10.5, 11, 11.5, 12, 12.5, 13, 13.5, 14, 14.5, 15, 15.5, 16, 16.5, 17, 17.5, 18, 18.5, 19, 19.5, or about 20 μW. In other embodiments, the intensity of the light pulses is about 20, 21, 22, 23, 24, 25, 26, 27, 28, 29, 30, 31, 32, 33, 34, or about 35 μW. In additional embodiments, the intensity of the light pulses is about 30, 31, 32, 33, 34, 35, 36, 37, 38, 39, 40, 41, 42, 43, 44, or about 45 μW. In yet other embodiments, the intensity of the light pulses is about 40, 41, 42, 43, 44, 45, 46, 47, 48, 49, or about 50 or more μW. In a preferred embodiment, the intensity of the light pulses is about 0.7, 0.75, or about 0.8 μW. In another preferred embodiment, the intensity of the light pulses is about 2, 2.5, or about 3 μW. In yet another preferred embodiment, the intensity of the light pulses is about 2, 2.5, or about 3 μW. In another preferred embodiment, the intensity of the light pulses is about 4, 4.5, 5, 5.5, or about 6 μW. In still another preferred embodiment, the intensity of the light pulses is about 26, 27, or about 28 μW. In another preferred embodiment, the intensity of the light pulses is about 11.5, 12, 12.5, 13, or about 13.5 μW. In an exemplary embodiment, the intensity of the light pulses is about 5 μW. In another exemplary embodiment, the intensity of the light pulses is about 27 μW. In yet another exemplary embodiment, the intensity of the light pulses is about 12.5 μW. In another exemplary embodiment, the intensity of the light pulses is about 5 μW.

In other embodiments, an artificial optical input may be adjusted by controlling the artificial optical input dwell time. The artificial optical input dwell time may be about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, or about 150 μs or more. In some embodiments, an artificial optical input dwell time is about 1, 5, 10, 15, 20, 25, 30, or about 35 μs. In other embodiments, an artificial optical input dwell time is about 30, 35, 40, 45, 50, 55, 60, or about 65 μs. In yet other embodiments, an artificial optical input dwell time is about 60, 65, 70, 75, 80, 85, 90, or about 95 μs. In still other embodiments, an artificial optical input dwell time is about 90, 95, 100, 105, 110, 115, 120, 125, 130, or about 135 μs. In other embodiments, an artificial optical input dwell time is about 130, 135, 140, 145, or about 150 μs. In an exemplary embodiment, an artificial optical input dwell time is about 75, 80, or about 85 μs.

To create an artificial optical input, an optical signal may be directed to a cell expressing an exogenous opsin (e.g. directed to the cell periphery) or adjacent to a cell expressing an exogenous opsin. In some embodiments, an artificial optical signal is directed to a cell expressing an exogenous opsin. In other embodiments, an artificial optical signal is directed to an area adjacent to a cell expressing an exogenous opsin. In general, the distance of an artificial optical signal from the cell periphery is directly proportional to the area of the artificial optical input. For instance, when a cell is a macrophage and the cell behavior controlled by optical activation is cell migration, an artificial optical input may be about 1, about 5, about 10, about 15, about 20, about 25, about 30, about 35, about 40, about 45, about 50, about 55, about 60, about 65, about 70, about 75, about 80, about 85, about 90, about 95, or about 100% of the size of the macrophage, and an artificial optical signal is about 1, about 2, about 3, about 4, about 5, about 6, about 7, about 8, about 9, about 10, about 11, about 12, about 13, about 14, or about 15 μm away from the cell periphery. In some embodiments, when the cell is a macrophage and the cell behavior controlled by optical activation is cell migration, an artificial optical input may be about 5, about 6, about 7, about 8, about 9, about 10, about 11, about 12, about 13, about 14, about 15, about 16, about 17, about 18, about 19, about 20, about 21, about 22, about 23, about 24, or about 25% of the size of the macrophage, and the optical signal is about 1, about 2, about 3, about 4, about 5, about 6, about 7, or about 8 μm away from the cell periphery. In other embodiments, when the cell is a macrophage and the cell behavior controlled by optical activation is cell migration, an artificial optical input may be about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, or about 45% of the size of the macrophage, and an artificial optical signal is about 1, about 2, about 3, about 4, about 5, about 6, about 7, about 8, about 9, about 10, about 11, about 12, about 13, about 14 or about 15 μm away from the cell periphery. In an exemplary embodiment, when the cell is a macrophage having an area of 528 μm², the artificial optical input is 9.4 μm² and the optical signal is 4 μm away from the cell periphery. In an exemplary embodiment, when the cell is a macrophage having an area of 528 μm², the artificial optical input is 9.4 μm² and the optical signal is 5 μm away from the cell periphery.

When a cell is a neuron and the cell behavior controlled by optical activation is initiation of a neurite, neurite outgrowth or extension of a neurite, an artificial optical input may be smaller than the neurite tip, and an optical signal is about 1, about 2, about 3, about 4, about 5, about 6, about 7, about 8, about 9, about 10, about 11, about 12, about 13, about 14, or about 15 μm away from the neurite tip. In preferred embodiments, when a cell is a neuron and the cell behavior controlled by optical activation is initiation of neurite or extension of a neurite, the optical input may be smaller than the neurite tip, and the optical signal is about 1, about 2, about 3, about 4, about 5, about 6, about 7, or about 8 μm away from the neurite tip.

When the cell is a cardiomyocyte and the cell behavior controlled by changing an artificial optical input is cardiomyocyte contractility, an artificial optical input may be covering the entire cell or multiple cells, or the area may be as described above. The frequency of an artificial optical input for controlling cardiomyocyte behavior may be similar to that described above.

One or more optical signals may be used to create a plurality of artificial optical inputs. Methods of the invention may use 1, 2, 3, 4, 5, 6, 7, 8, 9 or 10 artificial optical inputs. In some embodiments, 1, 2, 3, or 4 artificial optical inputs are used. In other embodiments, 4, 5, 6, or 7 artificial optical inputs used. In yet other embodiments, 7, 8, 9 or 10 artificial optical inputs are used. In a preferred embodiment, one artificial optical input is used. In another preferred embodiment, more than one artificial optical input is used.

When more than one artificial optical input is used, the artificial optical inputs may be created simultaneously or sequentially. In some embodiments, the more than one artificial optical inputs are created simultaneously. In other embodiments, the more than one artificial optical inputs are created sequentially.

In some embodiments, when more than one artificial optical input is used each input is restricted to a distinct location on the cell's surface. In certain embodiments, a plurality of artificial optical inputs may activate one exogenous opsin expressed in a cell at distinct location on the cell's surface. In certain other embodiments, a cell comprising more than one exogenous opsin, each comprising a different light sensing domain, and a plurality of artificial optical inputs at different wavelengths may be used to independently activate each exogenous opsin expressed in a cell.

When more than one artificial optical input is created to activate more than one exogenous opsin, each comprising a different light sensing domain, the exogenous opsins may be capable of activating one or more than one G protein subtype to activate one or more than one downstream signaling cascade to steer cellular behavior. In one embodiment, the exogenous opsins are capable of activating one G protein subtype to activate one downstream signaling cascade to steer cellular behavior. Stated another way, though each exogenous opsin has a different light sensing domain, the GPCR activation domain of each opsin activates the same G protein subtype. In another embodiment, the exogenous opsins are capable of activating more than one G protein subtype to activate more than one downstream signaling cascade to steer cellular behavior. Stated another way, each exogenous opsin has a different light sensing domain and each GPCR activation domain of each opsin activates a different G protein subtype.

The spatially confined activation of an exogenous opsin leads to asymmetric signaling and spatially controlled cell behavior. In some preferred embodiments, when a cell is a macrophage cell expressing an exogenous blue opsin, initiating spatially controlled G protein signaling leads to migration of the macrophage cell towards the artificial optical input. In other preferred embodiments, when a cell is a neuron cell expressing an exogenous blue opsin, initiating spatially controlled G protein signaling induces development and growth of a neurite from the cell periphery in the direction of the artificial optical input.

The artificial optical input may be temporally controlled. For instance, the artificial optical input may be temporally controlled by illuminating the artificial optical input for a duration of time sufficient to activate the opsin in the cell adjacent to the artificial optical input and achieve the desired spatially controlled cell behavior. In some preferred embodiments, when the cell is a macrophage cell, spatiotemporal control leads to asymmetric signaling and controlled migration of the macrophage cell towards the artificial optical input. As described in the examples, as the cell moves towards the optical stimulation area, the artificial optical input may be moved away from the cell periphery to maintain the distance of the artificial optical input from the cell periphery described above. Using the method of the invention, a macrophage may be moved any distance in any desired direction by moving the artificial optical input a desired distance in the desired direction. As demonstrated in the examples, continual movement of the artificial optical input is needed to maintain cell migration.

In other preferred embodiments, when a cell is a neuron cell, initiating spatially controlled signaling initiates neurite growth and growth of a neurite from the cell periphery. In some embodiments, neurite growth is initiated. In other embodiments, neurite growth is initiated. As used herein, the terms “neurite growth”, “neurite outgrowth” and neurite extension” are used interchangebly. As described in the examples, when an artificial optical input is adjacent to a neuronal cell periphery, neurite growth may be initiated. When an artificial optical input is adjacent to an already existing neurite tip, neurite growth may be initiated. Using the method of the invention, a neurite growth may be initiated and extended in any desired direction by moving the artificial optical input a desired distance in the desired direction. As demonstrated in the examples, once neurite growth is initiated, the artificial optical input can be removed and neurite outgrowth will continue. In some embodiments, pulse frequency is as described above and pulse length will be (optical area in μm²)×0.87 msec/μm². In some embodiments, artificial optical input duration may be for about 1, about 2, about 3, about 4, about 5, about 6, about 7, about 8, about 9, about 10, about 11, about 12, about 13, about 14 or about 15 minutes. In other embodiments, artificial optical input duration may be greater than 15 minutes.

The extracellular domain of opsin binds retinal, a photoreactive chromophore. Under certain circumstances, for instance when an opsin is introduced into a cell that normally does not comprise an opsin, retinal may be provided to generate a functional opsin in the cell. In particular, addition of retinal may be needed when methods of the invention are practiced in cell culture (e.g. in vitro). Methods of providing retinal to a cell are known in the art, and may include addition of the retinal to the cell culture medium. When the cell is in a culture medium, retinal may be continuously present in the culture medium of the cell, or may be added prior to optical activation of the opsin. In some embodiments, when the cell is in a culture medium, retinal is continuously present in the culture medium. In preferred embodiments, when the cell is in a culture medium, retinal is added prior to optical activation of the opsin. Retinal may be added 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, about 60 minutes or about 1.5, 2, 2.5, or 3 hours prior to optical activation of the opsin. In some embodiments, retinal is added 1, 2, 3, 4, 5, 6, 7, 8, 9, 10, 11, 12, 13, 14, 15, 16, 17, 18, 19, or about 20 minutes prior to optical activation of the opsin. In other embodiments, retinal is added 15, 16, 17, 18, 19, 20, 21, 22, 23, 24, 25, 26, 27, 28, 29, 30, 31, 32, 33, 34, or about 35 minutes prior to optical activation of the opsin. In yet other embodiments, retinal is added 30, 31, 32, 33, 34, 35, 36, 37, 38, 39, 40, 41, 42, 43, 44, 45, 46, 47, 48, 49, or about 50 minutes prior to optical activation of the opsin. In additional embodiments, retinal is added 45, 46, 47, 48, 49, 50, 51, 52, 53, 54, 55, 56, 57, 58, 59, or about 60 minutes prior to optical activation of the opsin. In preferred embodiments, retinal is added 25, 26, 27, 28, 29, 30, 31, 32, 33, 34, or about 35 minutes prior to optical activation of the opsin.

As will be appreciated by a skilled artisan, the concentration of retinal that may be added to the cell culture can and will vary depending on the cell type, level of opsin expression in the cell, and culture conditions and may be experimentally determined. For instance, the concentration of retinal that may be added to the cell culture may be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, or about 6 ng/ml of culture medium. In some embodiments, the concentration of retinal that may be added to the cell culture may be about 0.1, 0.2, 0.3, 0.4, 0.5, 0.6, 0.7, 0.8, 0.9, 1, 1.1, 1.2, 1.3, 1.4, or about 1.5 ng/ml of culture medium. In other embodiments, the concentration of retinal that may be added to the cell culture may be about 1, 1.1, 1.2, 1.3, 1.4, 1.5, 1.6, 1.7, 1.8, 1.9, 2, 2.1, 2.2, 2.3, 2.4, or about 2.5 ng/ml of culture medium. In yet other embodiments, the concentration of retinal that may be added to the cell culture may be about 2, 2.1, 2.2, 2.3, 2.4, 2.5, 2.6, 2.7, 2.8, 2.9, 3, 3.1, 3.2, 3.3, 3.4, or about 3.5 ng/ml of culture medium. In additional embodiments, the concentration of retinal that may be added to the cell culture may be about 3, 3.1, 3.2, 3.3, 3.4, 3.5, 3.6, 3.7, 3.8, 3.9, 4, 4.1, 4.2, 4.3, 4.4, or about 4.5 ng/ml of culture medium. In other embodiments, the concentration of retinal that may be added to the cell culture may be about 4, 4.1, 4.2, 4.3, 4.4, 4.5, 4.6, 4.7, 4.8, 4.9, 5, 5.1, 5.2, 5.3, 5.4, or about 5.5 ng/ml of culture medium. In other embodiments, the concentration of retinal that may be added to the cell culture may be about 5, 5.1, 5.2, 5.3, 5.4, 5.5, 5.6, 5.7, 5.8, 5.9, or about 6 ng/ml of culture medium. In preferred embodiments, the concentration of retinal that may be added to the cell culture may be about 2.5, 2.6, 2.7, 2.8, 2.9, 3, 3.1, 3.2, 3.3, 3.4, or about 3.5 ng/ml of culture medium.

(E) Introduction into a Cell

According to the invention, an exogenous opsin may be introduced into a cell. As used herein, the term “introduced into a cell” may refer to any method that may lead to expression of an exogenous opsin in a cell. Non-limiting examples of methods of introducing an exogenous opsin into a cell may include introducing an amino acid sequence comprising an opsin (i.e. an opsin protein) into the cell, and introducing a nucleic acid sequence capable of expressing an opsin in the cell.

i. Protein Transfection.

In some embodiments, an amino acid sequence comprising an exogenous opsin may be introduced into a cell. Methods of introducing an amino acid sequence or a protein into a cell are known in the art. For instance, an exogenous opsin may be introduced into a cell by injection, using a cell, a vesicle or a cell targeting peptide, or by transfection into the cell via a protein transfection agent. In one embodiment, an opsin may be introduced into a cell by injection into the cell. In another embodiment, an opsin may be introduced into a cell using a vesicle. Vesicles may be as described further below. In yet another embodiment, an opsin may be introduced into a cell via a protein transfection agent. Non-limiting examples of protein transfection agents may include the Influx® pinocytic cell-loading agent, the BIOPORTER® transfection agent, the Pierce protein transfection reagent, the TransPass P Protein Transfection Reagent, the Chariot Protein Delivery Reagent, the ProteoJuice™ Protein Transfection Reagent, the Xfect Protein Transfection Reagent, the Lipodin-Pro™ Protein Transfection Reagent, BioPORTER® Protein Delivery Reagent, PULSIN™. In still another embodiment, an opsin may be introduced into a cell by creating a fusion protein comprising a cell targeting peptide and an opsin, and introducing the fusion protein into the cell.

An exogenous opsin may be purified before introduced into a cell. Methods of purifying proteins are generally known in the art of protein biochemistry. For example, polypeptides may be purified via standard methods including electrophoretic, molecular, immunological and chromatographic techniques, ion exchange, hydrophobic, affinity, and reverse-phase HPLC chromatography, and chromatofocusing. As another example, polypeptides may be purified from the flow through of reverse-phase beads. Ultrafiltration and diafiltration techniques, in conjunction with protein concentration may also be used. For general guidance in suitable purification techniques, see Scopes, R., Protein Purification, Springer-Vertag, NY (1982).

ii. Transfection of Nucleic Acid Expressing an Exogenous Opsin

In some embodiments, an exogenous opsin may be introduced into a cell by introducing into the cell a nucleic acid sequence capable of expressing the exogenous opsin. In short, an expression construct may be constructed that generates an opsin when expressed in the cell after being introduced into the cell. A promoter may regulate the expression of a nucleic acid sequence constitutively or differentially with respect to the cell, the tissue or organ in which expression occurs or, with respect to the developmental stage at which expression occurs, or in response to external stimuli such as physiological stresses, pathogens, metal ions, or inducing agents or activators (i.e. an inducible promoter).

Methods of making an expression construct are known in the art. In brief, a nucleic acid sequence encoding the opsin is operably linked to a promoter. The term promoter, as used herein, may mean a synthetic or naturally-derived molecule which is capable of conferring or activating expression of a nucleic acid sequence in a cell. The promoter may be the promoter normally associated with the nucleic acid sequence encoding an opsin, or may be a heterologous promoter. A heterologous promoter may be derived from such sources as viruses, bacteria, fungi, plants, insects, and animals.

The promoter may be constitutive or inducible. Non-limiting examples of inducible promoters may include promoters induced by the presence of a small molecule (e.g., IPTG, galactose, tetracycline, steroid hormone, abscisic acid), a metal (e.g., copper, zinc, cadmium), an environmental factor (e.g., heat, cold, stress), and the expression of an exogenous protein (e.g., T7 RNA polymerase, SP6 RNA polymerase). Non-limiting examples of a constitutive promoter may include beta-actin promoter, cytomegalovirus intermediate-early (CMV) promoter, Rous sarcoma virus (RSV) promoter, simian virus 40 early (SV40) promoter, ubiquitin C promoter, elongation factor 1 alpha (EF1α) promoter, a promoter comprising the tetracycline response element (TRE) nucleic acid sequence, and the CMV IE promoter, and combinations thereof.

In some embodiments, an expression system may further comprise a transcription termination sequence. A transcription termination sequence may be included to prevent inappropriate expression of nucleic acid sequences adjacent to the heterologous nucleic acid sequence.

All the nucleic acid sequences of the invention may be obtained using a variety of different techniques known in the art. The nucleotide sequences, as well as homologous sequences, may be isolated using standard techniques, purchased or obtained from a repository. Once the nucleotide sequence is obtained, it may be amplified for use in a variety of applications, using methods known in the art.

In some embodiments, an expression system may be incorporated into a vector. One of skill in the art would be able to construct a vector through standard recombinant techniques (see, for example, Sambrook et al., 2001 and Ausubel et al., 1996, both incorporated herein by reference). Vectors include but are not limited to, plasmids, cosmids, transposable elements, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

A nucleic acid encoding an opsin may also be operably linked to a nucleotide sequence encoding a selectable marker. A selectable marker may be used to efficiently select and identify cells that have integrated the exogenous nucleic acids. Selectable markers give the cell receiving the exogenous nucleic acid a selection advantage, such as resistance towards a certain toxin or antibiotic. Suitable examples of selectable markers that confer antibiotic resistance include, but are not limited to, puromycin resistance gene (pac), neomycin resistance gene, hygromycin resistance gene, phlebomycin resistance gene, and blasticidin resistance gene. These genes encode for proteins that impart resistance to antibiotics such as puromycin, geneticin (G418), hygromycin, zeocin, and blasticidin, respectively. In a preferred embodiment, the operably linked antibiotic resistance gene may be pac, which encodes resistance to puromycin.

An expression construct encoding an opsin may be delivered to the cell using a viral vector or via a non-viral method of transfer. Viral vectors suitable for introducing nucleic acids into cells include retroviruses, adenoviruses, adeno-associated viruses, rhabdoviruses, and herpes viruses. Non-viral methods of nucleic acid transfer include naked nucleic acid, liposomes, and protein/nucleic acid conjugates. An expression construct encoding opsin that is introduced to the cell may be linear or circular, may be single-stranded or double-stranded, and may be DNA, RNA, or any modification or combination thereof.

An expression construct encoding opsin may be introduced into the cell by transfection. Methods for transfecting nucleic acids are well known to persons skilled in the art. Transfection methods include, but are not limited to, viral transduction, cationic transfection, liposome transfection, dendrimer transfection, electroporation, heat shock, nucleofection transfection, magnetofection, nanoparticles, biolistic particle delivery (gene gun), and proprietary transfection reagents such as Lipofectamine, Dojindo Hilymax, Fugene, jetPEI, Effectene, or DreamFect. Nanoparticles may be as described further below.

Upon introduction into the cell, an expression construct encoding an opsin may be integrated into a chromosome. In some embodiments, integration of the expression construct encoding an opsin into a cellular chromosome may be achieved with a mobile element. The mobile element may be a transposon or a retroelement. A variety of transposons are suitable for use in the invention. Examples of DNA transposons that may be used include the Mu transposon, the P element transposons from Drosophila, and members of the Tcl/Mariner superfamily of transposons such as the sleeping beauty transposon from fish. A variety of retroelements are suitable for use in the invention and include LTR-containing retrotransposons and non-LTR retrotransposons. Non-limiting examples of retrotransposons include Copia and gypsy from Drosophila melanogaster, the Ty elements from Saccharomyces cerevisiae, the long interspersed elements (LINEs), and the short interspersed elements (SINEs) from eukaryotes. Suitable examples of LINEs include L1 from mammals and R2Bm from silkworm.

Integration of the exogenous nucleic acid into a cellular chromosome may also be mediated by a virus. Viruses that integrate nucleic acids into a chromosome include adeno-associated viruses and retroviruses. Adeno-associated virus (AAV) vectors may be from human or nonhuman primate AAV serotypes and variants thereof. Suitable adeno-associated viruses include AAV type 1, AAV type 2, AAV type 3, AAV type 4, AAV type 5, AAV type 6, AAV type 7, AAV type 8, AAV type 9, AAV type 10, and AAV type 11. A variety of retroviruses are suitable for use in the invention. Retroviral vectors may either be replication-competent or replication-defective. The retroviral vector may be an alpharetrovirus, a betaretrovirus, a gammaretrovirus, a deltaretrovirus, an epsilonretrovirus, a lentivirus, or a spumaretrovirus. In a preferred embodiment, the retroviral vector may be a lentiviral vector. The lentiviral vector may be derived from human, simian, feline, equine, bovine, or lentiviruses that infect other mammalian species. Non-limiting examples of suitable lentiviruses includes human immunodeficiency virus (HIV), simian immunodeficiency virus (SIV), feline immunodeficiency virus (FIV), bovine immunodeficiency virus (BIV), and equine infectious anemia virus (EIAV). In an exemplary embodiment, the lentiviral vector may be an HIV-derived vector.

Integration of an expression construct encoding an opsin into a chromosome of the cell may be random. Alternatively, integration of an expression construct encoding an opsin may be targeted to a particular sequence or location of a chromosome. In general, the general environment at the site of integration may affect whether the integrated expression construct encoding an opsin is expressed, as well as its level of expression.

Cells transfected with the expression construct encoding an opsin generally will be grown under selection to isolate and expand cells in which the nucleic acid has integrated into a chromosome. Cells in which the expression construct encoding an opsin has been chromosomally integrated may be maintained by continuous selection with the selectable marker as described above. The presence and maintenance of the integrated exogenous nucleic acid sequence may be verified using standard techniques known to persons skilled in the art such as Southern blots, amplification of specific nucleic acid sequences using the polymerase chain reaction (PCR), and/or nucleotide sequencing.

iii. Nanoparticles

Any of the methods of introducing an exogenous opsin into a cell described above may be introduced using a vehicle for cellular delivery. In these embodiments, typically a composition comprising an opsin is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the composition of the invention in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the composition of the invention may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the composition of the invention (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The composition of the invention may be encapsulated in a microemulsion by any method generally known in the art.

In yet another embodiment, a composition of the invention may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of an dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### II. Method of Modulating Localized G Protein Signaling in a Tissue

In another aspect, the present invention comprises a method of initiating localized G protein signaling in a tissue via an exogenous opsin. The method comprises introducing into a plurality of cells within a tissue an exogenous opsin which forms a complex with a G protein. The tissue expressing an opsin, or the plurality of cells expressing an opsin within the tissue may be exposed to a light source, such that the opsins are activated, thereby initiating signaling throughout the tissue via the opsin.

In another aspect, the present invention comprises a method of modulating localized G protein signaling in at least one cell in a tissue using an artificial optical input. The method comprises introducing into at least one cell within a tissue an exogenous opsin, wherein the exogenous opsin comprises a light sensing domain of a melanopsin or a metazoan color opsin and a G protein coupled receptor (GPCR) activation domain that affects G protein signaling; and (b) changing an artificial optical input in a localized region on the surface of at least one cell in the tissue comprising the exogenous opsin, wherein the activation state of the exogenous opsin within the localized region is affected when the extracellular light sensing domain detects a change in the artificial optical input, thereby resulting in the GPCR intracellular activation domain modulating G protein signaling in the cell in the tissue. Methods of modulating localized G protein signaling in a cell using an artificial optical input is described above in Section I.

In some embodiments, the cell is a neuron and the tissue is a nervous tissue. Modulating localized G protein signaling in at least one neuron expressing an exogenous opsin in nervous tissue using an artificial optical input may be used to overcome damage to one or more neurons in the tissue by optically stimulating neurite growth and forming new neuronal connections. In other embodiments, the cell is an immune cell and the tissue is a tumor. Modulating localized G protein signaling in at least one immune cell expressing an exogenous opsin in the bloodstream may be used to encourage (i.e. induce or promote) extravasation of the immune cells from the bloodstream to the tumor (i.e. initiate cell migration from the bloodstream to the tumor), where the immune cells may suppress tumor growth. Modulating localized G protein signaling in circulating immune cells in the bloodstream may be performed using an implanted miniaturized light source in the aggressive growth region of the tumor. In yet other embodiments, the cell is a pancreatic islet cell and the tissue is parenchymal tissue. Modulating localized G protein signaling in at least one pancreatic islet cell expressing an exogenous opsin using an artificial optical input may be used to modulate insulin secretion. In other embodiments, the cell is a cardiomyocyte and the tissue is cardiac tissue. Modulating localized G protein signaling in at least one cardiomyocyte expressing an exogenous opsin in cardiac tissue using an artificial optical input may be used to control contractility of cardiomyocytes and control the rate of beating cardiac tissue.

### EXAMPLES

The following examples illustrate various iterations of the invention.

### Introduction for Examples 1-9

GPCRs initiate most of the signaling in metazoans and regulate a wide variety of cellular responses that include differentiation, migration, secretion and contraction. Diffusible molecules with limited lifetimes activate most of these receptors. Thus single cells sense and respond to extracellular signals that vary in location, duration and intensity in varied processes such as cell polarization and neuron function. Furthermore, the complex single cell behavior that occurs in response to activation of GPCRs is a continuum of cellular events. To probe the control of these events by a signaling network, methods have to be developed to activate signaling restricted to a selected region of a single cell for defined durations of time. They should faithfully evoke the native molecular and cellular responses in their entirety. Additionally, they should facilitate quantitative monitoring of response dynamics continually in a single cell.

Microfluidic devices have been used to regulate cellular behavior that occurs in response to GPCR activation but it is difficult to provide continually varying spatially and temporally discrete inputs and monitor a series of distinct responses using this approach. Experimental requirements are also cumbersome. Optical methods can overcome these limitations because they can continually provide varying input, spatial confinement to create asymmetry and the ability to switch signal input in space and time almost instantaneously.

In the Examples detailed below, it is examined whether the evolutionary conservation in specific coupling of G protein types with diverse GPCRs will allow entire signaling networks in heterologous cell types to be activated by non-rhodopsin visual opsins and if activation can be confined to a restricted region of a single cell. It is further examined if asymmetric signaling activity introduced by optically localizing receptor activation within a cell can help control the behavior of the cell. The non-rhodopsin visual opsins are a large family of receptors that are spectrally tuned to wavelengths that span the entire visual spectrum and are selectively capable of activating all the major G protein types and second messengers. They are used as the basis for building a family of genetically encoded optical triggers of signaling.

In the Examples below, these optical triggers, which selectively activate all major second messenger pathways in restricted areas of a single cell are described. Optical methods that allow the behavior of opsin-expressing cells to be controlled continually while cellular, cytoskeletal and signaling responses are imaged are also described. Existing optical methods are not designed to accomplish these tasks. Rhodopsin and its chimeric forms have been previously shown to activate different G protein types in mammalian cells and modulate critical neuronal activity. However, rhodopsin based optical activation is not well suited for repeated and sustained activation of a spatially restricted part of a single cell. For instance, a Gs coupled rhodopsin form was not able to evoke reproducible continuous signaling activity due to bleaching. Also, rhodopsin based triggers do not provide spatially confined activation likely due to the high sensitivity and slow deactivation of rhodopsin compared to color opsins. Optically induced cell protrusion or migration has been shown using Rho proteins with a light sensitive domain insertion. While valuable for probing Rho specific activity, this method cannot be used to optically control and observe entire signaling networks including critical second messengers. It is also not applicable to diverse cellular events that are GPCR modulated but not Rho dependent. In contrast, the optical triggers described in the Examples below, are cell surface receptors that sense extracellular optical inputs and activate all native GPCR signaling networks in their entirety.

Since global activation of GPCRs in a neuron induces neurite outgrowth, the ability of the approach described below was validated to provide spatiotemporally discrete inputs to a single cell and evoke specific responses by examining early neuron differentiation events. Optical control over patterns of neurite growth from a hippocampal neuron was demonstrated. These methods were then used to identify the signaling network properties that govern an important GPCR mediated cell behavior, immune cell migration. Differential spatial activation of GPCRs across the cell mediates the migration of immune cells, invasive cancer cells and cells undergoing morphogenesis. Models of migration have been difficult to test experimentally and longstanding questions have remained about the internal guidance cue for migration, how the steep internal gradients required for migration are created and the network properties that govern dynamic migratory behavior. The Examples below demonstrate that systematic and selective optical GPCR activation of a cell, in time and space variant patterns, can be used to steer cell migration precisely in any specified direction. Since the opsin-based trigger stimulates an entire and intact G protein-signaling pathway, this method is not pathway disruptive. It is capable of providing a read-out of all cellular and molecular events downstream of the stimulus. It is shown that the ability to monitor single cell responses to varied input series allows the signaling network dynamics to be interrogated and quantified. This helped test mathematical models to identify systems level control at the basis of migration initiation.

The optical approach described in the Examples also allowed the examination of whether there are differences between single cells in their response to the same extracellular signal and if such differences are reflected in their signaling network properties. Cell populations have traditionally been studied to understand the basis of biological function. Single cells within a population are however, likely to differ significantly in their properties. Information about this heterogeneity can help achieve a better understanding of the mechanisms underlying cell function. One of the limitations in probing single cell signaling networks has been the dearth of methods to control single cell behavior and monitor signaling dynamics. The optical approach described in the Examples below overcomes this limitation and allows the identification of dynamically changing network properties that underlie migratory behavior of a single cell.

### Example 1

### Development of Methods for Measuring Localized G Protein Activity within a Cell and Identification of an Optical Trigger of Gi

To activate signaling at subcellular resolution, an activated receptor should diffuse slowly and deactivate rapidly in the absence of the signal thus curtailing the spread of signaling activity. It was first examined if the human color opsins can provide localized activation because as transmembrane receptors they were expected to diffuse relatively slowly along the plasma membrane, and they are known to deactivate rapidly in their native cone photoreceptor environment.

The three human color opsins, blue, green and red have been identified and characterized biochemically, but their ability to function heterologously in an intact cell has not been examined. Color opsins are coupled to the G protein subunit, Gαtc in the cone photoreceptor cells of the mammalian retina. Since Gαtc is homologous to and falls in the Gi subfamily, it was examined if the human color opsins, blue, green and red, activate endogenous Gi signaling activity in HeLa cells. These opsins absorb maximally at 414 nm (blue), ˜540 nm (green) and ˜560 nm (red) (FIG. 1A).

An assay based on G translocation to detect GPCR activity confined to a selected region of a single cell was developed. Gβγ subunits translocate away from the plasma membrane to internal membranes on receptor activation and reverse on deactivation making translocation a direct indicator of receptor state. Here, G protein translocation away from the plasma membrane is leveraged to quantify both the spatial and temporal precision of optically triggered GPCR activity. The assay possesses the following characteristics. (i) It is a direct measure of GPCR activation and deactivation. (ii) It is a read-out of spatially restricted GPCR activation with high time resolution. We used Gγ9 tagged with a fluorescent protein (FP), as it is a rapidly translocating γ subunit (t_{1/2}˜10 s).

Initial characterization of the color opsins showed that all three opsins were capable of activating βγ translocation in a cell (FIG. 1B, C, and FIGS. 2A and B). It was then examined if increasing the number of light pulses could control the magnitude of G protein activation. Activation was measured by observing the extent of Gγ translocation. A repeating-pulse optical input was chosen over a continuous one to extend the lifespan of an activated opsin. The intensity of 445-nm optical input was titrated on a single cell expressing bOpsin-mCh and YFP-γ9 to determine the optimum intensity for optical activation. Increasing the beam intensity of optical inputs in a single cell increased the magnitude of YFP-γ9 translocation that reaches saturation at ˜5 μW (FIG. 10). A similar single-cell experiment at 5 μW showed increasing response to different number of light pulses (FIG. 1E). The results demonstrate that increasing the number of pulses increases both magnitude and duration of translocation. Thus, both light intensity and number of pulses can be used to modulate GPCR activity in a single cell.

The optical input to optimally activate blue opsin was designed by varying the size of the optical input area, the intensity of the beam, pixel dwelling time, and pulse frequency (FIGS. 1F and G). To achieve better control over spatially restricted signaling activity, the extent of confinement of GPCR activity was experimentally determined using optical activation of an opsin in a single cell. To do so, we exposed a confined region of the plasma membrane of a bOpsin-expressing cell to an optical input of 3-μm width. We measured GFP-γ9 loss in the plasma membrane 5 s after activation and found that this follows a Gaussian distribution with a full width at half maximum (FWHM)=6.3 (FIGS. 1H and 2C). This indicates that an optical input can induce confined activation with a steep gradient of decreasing activity at the boundary. These results showed that GPCR activity is restricted to the optical input area and this area can be a relatively small fraction of the cell surface.

After localized activation of green or red opsin, the basal state of the cell or the dynamics of the response could not be captured continually because wavelengths used to excite fluorescent proteins also activated these opsins globally. An opsin suitable for achieving confined signaling activity should be spectrally selective and not be activated during global imaging of the cell's response dynamics (FIG. 3A). To identify such an opsin various opsin expressing cells were screened by imaging under different wavelengths of light (FIG. 3B, Table 1). The laser intensities were titrated for optical activation and imaging down to appropriately low levels (FIG. 4A-D), so that a spectrally selective opsin could be identified. Such spectral selectivity was achieved by using blue opsin (bOpsin) (λmax 414 nm) and it was possible to sustain localized activation while imaging fluorescent proteins (FPs) over the entire cell with excitation wavelengths 488 nm. Localized bOpsin activation by restricting the laser beam (445 nm, ≧5 μW) to a limited area of a single cell resulted in localized βγ translocation away from the plasma membrane that could be imaged (FIG. 4C). Imaging by excitation at specified intensities of GFP (488 nm, <3 μW) and mCherry (mCh) (595 nm, <40 μW), did not activate bOpsin over the entire cell (FIG. 4D). This provided a unique platform for imaging basal level cellular activities without activating opsin. Pertussis toxin treatment inhibited Gβγ translocation induced by bOpsin activation showing that bOpsin activates the endogenous G protein, Gi in HeLa cells as anticipated.

TABLE 1
(Related to FIG. 1, 2) Characterization of opsins' spectral
selectivity using Gβγ9 translocation

| | | | | | | Jelly- | |
|---|---|---|---|---|---|---|---|
| Wave- | Green | Red | Blue | Melanop- | Rhodopsin | fish | |
| length | opsin | opsin | Opsin | sin | chimera | opsin | CrBlue |
| 595 | ✓ | ✓ | x | x | ✓ | ✓ | x |
| 515 | ✓ | ✓ | x | ✓ | ✓ | ✓ | x |
| 488 | ✓ | ✓ | x | ✓ | ✓ | ✓ | x |
| 445 | ✓ | ✓ | ✓ | ✓ | ✓ | ✓ | ✓ |

Using the method explained in FIG. 3, cells expressing individual opsins were screened at different wavelengths for FP-Gβγ9 translocation. Sign ✓ shows translocation and x the lack of translocation. Wavelengths that showed no translocation response were used to image fluorescence while translocation evoking wavelengths were employed to induce localized opsin activity.

It was further examined whether bOpsin could be used to achieve tight temporal control of G protein activation. Since forward and reverse translocation of Gβγ is highly sensitive to the activation and deactivation states of a receptor, bOpsin was stimulated by a single OA pulse and βγ translocation observed. Maximal βγ translocation was reached rapidly before reversal occurred (t_{1/2}˜1.7 s) (FIG. 3E) suggesting that bOpsin deactivates rapidly. This facilitates confinement of G protein activation to the optically stimulated region of the cell. Furthermore, it was possible to activate bOpsin repeatedly without desensitization (FIG. 3F). This allows it to be used to control the behavior of the same cell over extended periods of time by sustaining signaling activity with repeated activation.

It was then examined if the optically evoked localized G protein activation is reflected in second messenger levels. PIP3 is known to be activated by Gi coupled GPCRs in immune cells. Spatially restricted OA of bOpsin in a single RAW 264.7 macrophage cell showed PIP3 increase precisely within the activated region (FIGS. 3G and H). Together, these results suggest that bOpsin functions as a highly controllable optical trigger of localized, reversible and repeatable Gi/o signaling pathway at a single cell level. Furthermore, the optical methods developed allow global imaging of the response dynamics at high time resolution.

In contrast to the color opsins, it was found that chimeric forms of rhodopsin that activate Gs or Gi, did not provide spatiotemporally confined G protein activity within a single cell. In cells expressing these constructs, FP-βγ9 had translocated at the very beginning of imaging and did not return to the plasma membrane even after 3-4 minutes in the dark.

### Example 2

### Optical Activation of Localized Gq Signaling within a Single Cell

To develop a comprehensive set of optical triggers to induce localized signaling of all the major G protein pathways in a single cell, Gq and Gs coupled opsins were identified. Melanopsin, a Gq coupled opsin expressed in a subset of mammalian retinal ganglion cells, (λ max ˜480 nm) (FIG. 1A) was identified. OA (488 nm, 27 μW) of melanopsin in a HeLa cell induced Gβγ translocation (t_{1/2}˜5 s) (FIGS. 5A and B). It was possible to activate melanopsin repeatedly, and the rapid reversal of Gγ9 translocation (15 s) showed that melanopsin deactivates rapidly (FIG. 5C). Melanopsin induced IP3 production only in optically activated cells (yellow box) with t_{1/2}˜3.5 s (FIGS. 5D and E). Optically confined G protein activation in a single cell is reflected in localized changes in the second messenger IP3 (FIGS. 3I and J). Localized OA, elicited a rapid increase in IP3 in the region proximal to OA (white box) compared to a distal region (Δt_{1/2}˜4 s). These results clearly show that melanopsin can be used to exercise spatial and temporal control over Gq signaling within a single cell.

### Example 3

### Reengineering Opsins to Obtain Specific Combinations of Spectral Tuning and G Protein Coupling

The box jellyfish, Carybdea rastonii, expresses an opsin that is Gs coupled (λ max ˜500 nm) (FIG. 1A). In HeLa cells, the jellyfish opsin activated mCh-γ9 translocation globally as soon as imaging of mCh in the cells was initiated (FIGS. 7A and B, Table 1).

It was examined whether jellyfish opsin could be redesigned to introduce spectral selectivity while retaining Gs coupling. The conservation in the structure of GPCRs has facilitated the design of chimeric receptors that alter specificity for the extracellular signal and G protein subtype. A chimeric opsin, CrBlue, containing the chromophore-binding region of bOpsin and the Gs coupling intracellular region of jellyfish opsin was synthesized (FIG. 7C). In contrast to jellyfish opsin, CrBlue was not activated by wavelengths used to excite mCh, YFP or GFP. It was activated by 445 nm light (FIGS. 7D and E). Localized OA of CrBlue resulted in spatially restricted translocation of mCh-γ9 (FIG. 3L). CrBlue induced cAMP increase consistent with Gs activation and was spectrally selective allowing the basal state of the cell to be imaged (FIG. 3N).

Apart from providing an optical trigger of localized Gs signaling, these results also showed that it is possible to reengineer opsins with specific combinations of spectral sensitivity and G protein specificity.

### Example 4

### Optical Control of Neurite Initiation

To examine if an optical trigger can be used to control complex single cell behavior, post natal 1-2 day old hippocampal neurons were used at stage I or later. It has previously been shown that exposure of neurons to neurotransmitters that stimulate Gi/o encourages neurite outgrowth but the effect of spatially selective Gi/o coupled receptor activity on a neuron has not been clear.

Gi coupled bOpsin-mCh was expressed in hippocampal neurons and selected regions at the periphery of stage I cells with no neurites was optically activated (445 nm, 5 μW). The results showed that continuous pulses of the optical input (yellow box) resulted in the neuron responding with a protrusion followed by the formation of extensive lamellipodia (FIG. 6A, FIGS. 7A and B). After optical activation was terminated, the lamellipodia consolidated into a neurite over a period of two hours.

Optically induced neurite initiation possessed all the important characteristics of native neurite growth. Neurons coexpressing bOpsin and a dominant negative RacT17N failed to respond to OA compared to neurons expressing the wild type Rac confirming that OA induced neurite initiation is mediated by a Rac mediated pathway. Optically induced neurite formation in GFP-β actin expressing neurons showed extensive remodeling of the actin cytoskeleton (FIG. 6B, FIG. 7C). Thus the initial lamellipodia formation, Rac dependence and actin remodeling, recapitulate native properties seen during spontaneous neurite growth. These results show that the optical approach developed here recruits the endogenous signaling network in the cell and executes behavioral changes that mimic native cell behavior.

### Example 5

### Optically Reprogramming Neurite Extension-Retraction Cycles to Refashion Differentiation of a Single Neuron

Next, the effect of an optical input series on later stage hippocampal neurons was examined. Gi/o coupled CXCR4 receptors are enriched at the leading edge of neurites and are known to promote neurite growth. Since bOpsin is coupled to Gi, it was anticipated that applying a series of optical inputs of individual neurite tips in a bOpsin expressing neuron might alter the pattern of neurite extension in a differentiating neuron. The neuronal response dynamics was quantitatively monitored.

It was found that OA of an existing neurite initiated lamellipodia growth in hippocampal neurons expressing bOpsin. 2-3 hrs after the termination of OA the lamellipodia consolidated into a newly extended neurite (FIG. 8C). The period of OA required to extend a neurite was short (≦12 mins). As in the case of neurite initiation above, in neurons coexpressing mGFP-actin with bOpsin, extension of the neurite was accompanied by actin remodeling mimicking spontaneous neurite growth (FIG. 8D, E). Demonstrating the spatial confinement of the optical input and subsequent GPCR activity, only the optically activated neurite responded.

We then examined if we could use the ability to spatially switch the optical input to different neurite tips to reconfigure the extension pattern of neurites in a single neuron. When neurite tips were sequentially activated in a single neuron, the results showed that extension of lamellipodia was accompanied by simultaneous retraction of a growth area in the same neuron (FIG. 6C, D). There is a strong negative correlation between proximal growth and distal retraction showing that the two events are tightly coupled (FIG. 6E, FIG. 8F).

The rate (˜0.05 μm/s) and direction of neurite extension closely corresponded with that of optical input movement (FIG. 6F). This showed that the appropriate signaling input functions could be created optically to guide directionally sensitive neurite growth. Overall these results established that continually variant optical inputs could be used to evoke sustained directionally sensitive responses from cells expressing an opsin (FIG. 6G). These experiments allowed the establishment of the pattern of optical input characteristics required for reprogramming the complex growth dynamics of neurites during early neuron differentiation. While the neuron executes a series of complex behavioral events in response to optical commands the dynamic changes in molecules can be monitored and quantitated (Table 2).

TABLE 2
Quantification of dynamic parameters of optically induced
Gi mediated single neurite extensions and corresponding
lamellipodia retractions in the same neuronal precursor

| | Neurite extension | | | Lamellipodia Retraction | | |
|---|---|---|---|---|---|---|
| | Neu- | Neu- | Neu- | Neu- | Neu- | Neu- |
| | rite 1 | rite 2 | rite 3 | rite 1 | rite 2 | rite 3 |
| Lᵢ | 22.4 | 80 | 18.2 | — | — | — |
| L_{f} | 31 | 99 | 37.1 | | | |
| T_{half} | 188 | 220 | 244 | 125 | 244 | 344 |
| Tₚₑₐₖ | 266 | 345 | 307 | 195 | 475 | 605 |
| K_{half, Lamellipodia} | 38 | 40 | 49 | 25 | 49 | 69 |
| T_{total-E/R} | 113 | 273 | 142 | 93 | 404 | 479 |
| V_{avg} | 0.007 | 0.003 | 0.006 | 0.01 | 0.002 | 0.002 |
| Vₘₐₓ | 0.014 | 0.016 | 0.017 | 0.038 | 0.037 | 0.023 |
| E | 38% | 24% | 103% | — | — | — |

Lᵢ (μm): Initial length of the neurite, L_{f}(μm): Final length of the neurite, T_{half} (sec): Time required to reach half maximal valueof lamellipodia growth/collapse respectively for neurite extensions and lamellipodia retractions, Tₚₑₐₖ (sec): Time required to reach maximum lamellipodia growth, K_{half, Lamellipodia} (maximal of lamellipodia growth, T_{total-E/R} (sec): Total time required to extend the neurite or retract the lamellipodia (measured as the time required to reach 99% of the maximum growth from 1% of the maximum growth), V_{avg}, (increase in normalized fluorescence/sec): Average growth rate of lameliipodia, Vₘₐₓ: Maximum growth rate of lamellipodia, E: Percentage extension of the neurite length ((L_{f} − Lᵢ) × 100/L_{i,}).

### Example 6

### Asymmetric Optical Activation of bOpsin Initiates and Directs Immune Cell Migration Continually

Optical methods to interrogate the network properties that govern cell migration were used. This system was chosen because the second messengers involved in migration have been identified, but little is known about systems level network control of dynamic migratory events in single cells.

Immune cells such as neutrophils and macrophages migrate towards the higher concentration of a chemoattractant in a gradient. This migration is known to be mediated by the asymmetric activation of Gi coupled GPCRs across the cell. Here it was examined whether an optical gradient across a bOpsin-expressing immune cell can mimic gradients of diffusible molecules. Gradients of diffusible molecules have been used to study cell migration and obtain valuable information. However, it is difficult to use these gradients to make a single cell execute all possible migratory events while continually obtaining quantitative information on the dynamic response in the same cell (FIG. 9A (a)). In contrast, as demonstrated with neurons above, localized optical signals have the potential to induce signaling asymmetry in an immune cell and allow a migratory response to be followed continually in the same cell (FIG. 9A (b)). Here the response of a mouse macrophage RAW 264.7 cell expressing bOpsin-mCh to confined optical inputs (5 μW pulses at 5 s intervals) was examined. Cells were imaged for mCh (595/630 nm) continuously. FIG. 9B show that cells responded with a protrusion followed by lamellipodia formation towards the optical stimulus (colored box). The optical signal was continually relocated to direct migration. Spatially discrete optical inputs allowed independent control of two different cells simultaneously, demonstrating that the cell response is signal specific.

It was then examined if a migrating cell is capable of reversing direction when the optical signal is relocated. These experiments were performed using DIC imaging to examine if migratory events can be imaged under white light. Switching the optical signal to the back of a migrating cell resulted in lamellipodia initiation at the back and retraction at the front followed by cell movement in the reverse direction (FIG. 9C). When the migration of a cell that reversed its direction with reference to optical input was tracked, it was clear that the cell migrated precisely along the optical input trajectory (FIG. 9D). The cell did not show random walk behavior in response to the optical input (FIG. 9E). There was no apparent difference between forward and reverse average migration velocities (˜5 μm/min) (FIG. 10A). Together, these results demonstrated that optical inputs can mimic chemoattractants but with much more precise spatial and temporal control.

### Example 7

### A Migrating Cell Adapts to a Stationary Optical Input

There is limited understanding of the basis of adaptation in cell migration. It was examined whether the ability to spatially and temporally control signal input would allow the imaging of cellular and internal responses as the cell adapts to the signal. A cell following a moving optical input gradually decreased its velocity when the input movement ceased and eventually stopped moving (FIGS. 9F and G). Immune cells are thus capable of adaptation similar to Dictyostelium cells. These results showed that optical control allows precise orchestration of migratory behavior and simultaneous imaging of individual responses so that they can be quantitated.

### Example 8

### Optical Control Identifies an Amplified Front to Back PIP3 Gradient Underlying Migration Initiation

PIP3 is known to accumulate at the leading edge of a migrating cell and is thought to be a mediator of cell migration (FIG. 11A). However, the PIP3 response dynamics across a single cell as it executes migration initiation, directional changes in migration and adaptation have been difficult to examine. The ability to rapidly induce directional changes in cell migration or adaptation using controlled optical functions described here, allowed the examination the PIP3 response dynamics continually in a cell.

A RAW cell coexpressing bOpsin-mCh and a PIP3 sensor Akt-PH-GFP was imaged after sequentially activating the front and the back optically (FIG. 11B, C). In the basal state, the cell was polarized randomly with PIP3 patches distributed along the cell periphery. On optical activation, PIP3 increased rapidly at the activated front. Importantly, PIP3 levels decreased almost simultaneously at the back (FIG. 11D). Further optical activation resulted in lamellipodia formation and migration. Switching of the optical input to the back of the migrating cell resulted in rapid PIP3 gradient reversal (FIG. 11C).

PIP3 loss at the back was initiated simultaneously with PIP3 increase at the front (FIG. 11D) suggesting rapid communication between the front and back modules of the cell. Activation induced PIP3 increase at the front of a cell has previously been observed. However, rapid concomitant decrease in PIP3 at the back has not been detected previously. Breakdown of PIP3 at the back of a cell provides a significantly amplified gradient (FIG. 11E). Reversing the optical gradient yields a five-fold steeper PIP3 decrease compared to that seen on termination of OA suggesting that there is direct communication between the front and back (FIG. 11F).

The ability to create a stationary optical input (FIG. 11G, H) helped us examine the effect of adaptation on the steep PIP3 gradient in a migrating cell. This shows that as a cell gradually stops migrating when the optical signal becomes stationary (100 s), the PIP3 gradient progressively collapses although the signal is still present. It clearly shows that the signaling asymmetry created by the PIP3 gradient is essential for the continued migration. The dissipation of the gradient when a significant part of the cell surface is exposed to light provides support for a local activation excitation model, which has not been directly tested because of limitations in methods to specifically localize signaling to parts of a single cell. Overall, these results show that the PIP3 dynamics demonstrate the properties of an internal guidance cue by responding specifically, differentially and rapidly to directional changes in stimulus or adaptation.

When migration was initiated with OA, localized actin remodeling was detected at the front of the cell (FIGS. 10B and C). This remodeling occurs on a similar time scale to that of PIP3 at the front of a cell, consistent with a PIP3-Rac-actin pathway mediating optically induced migration similar to that in response to chemoattractants. When a dominant negative Rac(T17N) or wild type Rac was introduced into bOpsin expressing RAW cells, OA induced migration was inhibited by Rac(T17N) but not Rac wt. This result showed that optically directed migration is mediated by a Rac mediated pathway similar to chemoattractant induced migration. Actin remodeling and Rac mediation further confirm that optically directed migration occurs through opsin recruitment of the endogenous signaling network and that it is identical to that induced by chemoattractants.

### Example 9

### PIP3 Formation is Ultrasensitive and Migration Initiation Occurs Midway Through the Switch Like PIP3 Response in Two Distinct Populations of Cells

Experiments were designed using the optical approach to test the predictions from the modeling. Identical continuous light pulses were directed at a single cell to initiate cell migration. The PIP3 response in 23 cells was individually and simultaneously monitored. The dynamic response of PIP3 as a function of number of light pulses during the migration initiation in each of the cells was examined and found that it demonstrated ultrasensitivity (Hill coefficients, n_{H}˜3-˜8) for individual cells (FIG. 12A) as predicted. The values for the Hill coefficients suggest that the PIP3 response is highly ultrasensitive. This switch like property of an ultrasensitive response is well suited to mediate decisive changes in cellular states.

To characterize the cell to cell variation in a population, the optical approach was used to examine the relationship between the number of light pulses required for the half maximal PIP3 response (K) and migration initiation (Nₛₜₐᵣₜ) in single cells. The sensitive nature of the experimental protocol yielded the unique K for different cells studied. These cells showed heterogeneity in both PIP3 response and migration initiation (FIG. 12B). But, strikingly, migration is initiated in almost all the cells close to the half maximal PIP3 response.

The Nₛₜₐᵣₜ and K for the 18 cells were fitted with a cumulative distribution which further yielded the normalized distribution (FIG. 12C). It is clear that the Nₛₜₐᵣₜ and K demonstrate a bi-modal distribution, with some cells having a lower threshold for activation and others having a higher value. Such a population level analysis could be carried out due to the precise evaluation of K in the different single cell experiments facilitated by the optical approach. The averaged PIP3 response of cells in these two populations was then obtained. The results provided two populations with distinct K values. (i) Early migrants (blue, K=37), required fewer stimuli for the initiation of migration and (ii) late migrants (red, K=67), which required more stimuli (FIG. 12D). In these populations, migration is initiated at the switch like region of the ultrasensitive PIP3 increase at the front.

Systems level analysis of a single migrating cell is difficult using present methods because it requires continuous control over the input function across a cell while simultaneously imaging cellular and molecular responses. The ability to control migration with confined optical input allowed quantitative determination of the PIP3 and migratory response as a function of varying stimulus (number of light pulses) in a single cell (FIG. 12E). This provided a capability to generate a mathematical model of the signaling network structure in a single migrating cell.

The ability to confine activation of receptor and coupled G proteins to one edge of a cell allowed us to develop a two-compartment ordinary differential equation model for PIP3 accumulation in immune cells that is mediated by asymmetric GPCR activation. The model assumed the basic framework of a local excitation, global inhibition mechanism. An inhibition-activation mechanism was introduced wherein there is antagonism between the inhibitor and the activator at the membrane as shown in FIG. 12F. The model thus comprises an incoherent feed-forward loop with faster activation kinetics of a membrane localized activator and slower recruitment kinetics of an inhibitor to the membrane from the cytosol. FIG. 12F shows the reaction schematic used for the formulation of the model, and Tables 3-5 describe other aspects of the model.

The activator enhances PIP3 synthesis and the inhibitor decreases PIP3 levels. The activator is confined to the plasma membrane whereas the diffusible inhibitor is present in both the membrane and the cytosol. Activation of the inhibitor leads to its membrane recruitment. The cytosolic inhibitor is capable of free exchange between the two compartments, whereasthe activator and the membrane-bound inhibitor remain localized.

The G protein activation rate is directly proportional to the stimulus (S). The free G protein concentration and deactivation of G protein follow first-order reaction kinetics. The total G protein concentration (GT) is constant in each compartment. The rate of accumulation of the activated G protein in the front compartment is

$$\frac{d {G}_{F}}{d t} = k 0 * {S}_{front} * ( Gt - {G}_{F} ) - k 0 r * {G}_{F} .$$

The rate of accumulation of activator is

$$\frac{d {A}_{mF}}{d t} = k 1 * \frac{{G}_{F}}{{G}_{F} + km} - k 1 r * {A}_{{m}_{F}} - k 2 r * {A}_{{m}_{F}} * {I}_{{m}_{F} .}$$

The rate of accumulation of the inhibitor in the cytosol in the front compartment is

$$\frac{d {I}_{cytF}}{d t} = k 2 * \frac{{G}_{F}}{{G}_{F} + km} + kt * ( {I}_{cytB} - {I}_{cytF} ) - krm * {I}_{vytF} .$$

The formation of PIP3 is first order with respect to activator, whereas the disappearance of PIP3 depends on both the inhibitor and its own concentration in the front compartment. The accumulation rate of PIP3 is

$$\frac{d PIP {3}_{F}}{d t} = k 30 + k 3 * {A}_{{m}_{F}} - ( k 3 r * {I}_{{m}_{F}} + k 30 r ) * PIP {3}_{F} .$$

Equations for the back compartment are

$$\frac{d {G}_{B}}{d t} = k 0 * {S}_{B} * ( Gt - {G}_{B} ) - k 0 r * {G}_{B} ;$$

$$\frac{d {A}_{mB}}{d t} = k 1 * \frac{{G}_{B}}{{G}_{B} + k m} - k 1 r * {A}_{{m}_{B}} - k 2 r * {A}_{{m}_{B}} * {I}_{{m}_{B}} ;$$

$$\frac{d {I}_{cytB}}{d t} = k 2 * \frac{{G}_{B}}{{G}_{B} + k m} - kt * ( {I}_{cytB} - {I}_{cytF} ) - krm * {I}_{cytB} ;$$

$$\frac{d {I}_{{m}_{B}}}{d t} = {V}_{c} * krm * {I}_{{cyt}_{B}} - k 1 r * {I}_{{m}_{B}} - k 2 r * {I}_{{m}_{B}} * {A}_{{m}_{B} ;}$$

$$\frac{d PIP {3}_{B}}{d t} = k 30 + k 3 * {A}_{{m}_{B}} - ( k 3 r * {I}_{{m}_{B}} + k 30 r ) * PIP {3}_{B} .$$

TABLE 3
Model variables

| Variable | Description |
|---|---|
| G_{F} | Activated G protein in front |
| A_{mF} | Activator at the membrane in front |
| I_{cytF} | Inhibitor in cytosol in front |
| I_{mF} | Inhibitor at the membrane in front |
| PIP3_{F} | PIP3 concentration in front |
| G_{B} | Activated G protein in back |
| A_{mB} | Activator at the membrane in back |
| I_{cytB} | Inhibitor in cytosol in back |
| I_{mB} | Inhibitor at the membrane in back |
| PIP3_{B} | PIP3 concentration in back |

TABLE 4
Model parameters used

| Parameter | Description | Value | Unit |
|---|---|---|---|
| S_{F} | Stimulus at front | 0.1 | Dimensionless |
| S_{B} | Stimulus at back | 0 | Dimensionless |
| k0 | G-protein activation | 0.04 | 1/s |
| k0r | G-protein deactivation | 0.02 | 1/s |
| k1 | Activator formation | 1.00 | μM/s |
| k2 | Inhibitor formation | 0.01 | μM/s |
| k1r | Activator and inhibitor deactivation | 0.2 | 1/s |
| krm | Inhibitor recruitment rate from | 0.25 | 1/s |
| | cytosol to membrane | | |
| k2r | Deactivation due to antagonism | 0.2 | 1/(μM * s) |
| k3 | PIP3 formation | 0.20 | 1/s |
| k3r | PIP3 disappearance | 0.20 | 1/(μM * s) |
| k30 | Basal PIP3 formation | 0.05 | μM/s |
| k30r | Basal PIP3 degradation | 0.05 | 1/s |
| kt | Inhibitor translocation | 10 | 1/s |
| km | Half-saturation constant for G | 0.50 | μM |
| | protein for activator and inhibitor | | |
| G_{T} | Total G protein | 0.5 | μM |
| V_{c} | Correction factor for effective | 100 | Dimensionless |
| | volumes corresponding to cytosol | | |
| | and membrane | | |

TABLE 5
Initial conditions of model components

| Initial conditions | Description | Value/μM |
|---|---|---|
| G_{F} | G protein at front | 0 |
| A_{mF} | Activator at membrane at front | 0 |
| I_{cytF} | Inhibitor in cytosol at front | 0 |
| I_{mF} | Inhibitor at membrane at front | 0 |
| PIP3_{F} | PI3 at front | 1 |
| G_{B} | Activated G protein in back | 0 |
| A_{mB} | Activator at the membrane in back | 0 |
| I_{cytB} | Inhibitor in cytosol in back | 0 |
| I_{mB} | Inhibitor at the membrane in back | 0 |
| PIP3_{B} | PIP3 concentration in back | 1 |

FIG. 12 G-I shows the simulated dynamic behavior of the activator and inhibitor. In the front compartment, both the activator and the cytosolic inhibitors increase. The rapid diffusion increases the concentration of the cytosolic inhibitor in both compartments. However, the lack of G-protein activation and consequent absence of activator at the back result in increasing inhibitor activity in the membrane at the back. The polarization of the activator and inhibitor between the two compartments results in a PIP3 gradient.

When signal input is switched to the back, G-protein activation induces the activator at the back, leading to deactivation of inhibitor at the back. Thus, the localized activity of activator and inhibitor gets reversed rapidly. Termination of input leads to the rapid decrease in the activator concentration at the “back”. However, in the absence of further switching of the optical signal to the opposite end of the cell, increased recruitment of the inhibitor to the back does not occur. Thus, the dissipation of the PIP3 gradient occurs at a rate that is slower than that in the presence of the asymmetric input as seen experimentally (FIGS. 11C and F). Overall, the simulation thus captures the kinetics of PIP3 changes across the cell observed when executing the experimental paradigm (FIG. 12I compared to 11C), thus validating the model.

Systems parameters for PIP3 response are presented below in Table 6.

TABLE 6

| | | |
|---|---|---|
| | | Pooled |
| Group 1 | Group 2 | population |

| Value | Mean | SEM | Mean | SEM | Mean | SEM |
|---|---|---|---|---|---|---|
| n_{H} | 3.9 | 0.4 | 4.4 | 0.6 | 4.2 | 0.4 |
| K | 38.7 | 4.2 | 77.2 | 9.2 | 64.4 | 7.6 |
| Nₚₑₐₖ | 63.2 | 5.0 | 86.6 | 5.4 | 79.2 | 4.7 |
| Nₛₜₐᵣₜ | 36.3 | 2.35 | 64.5 | 2.5 | 55.1 | 3.7 |

Pooled population: Entire population that shows migration. Grouped population: Grouping of cells on the basis of Nₛₜₐᵣₜ (the number of pulses required to initiate the cell migration). Values of mean and SEM for Hill coefficient (n_{H}), activation threshold (K), number of light pulses required to reach the peak PIP3 response (Nₚₑₐₖ), and Nₛₜₐᵣₜ are reported.

Overall these results show that individual cells in a population vary in their migratory responses based on critical differences in systems properties. In contrast to a population of cells studied using a diffusible gradient, the ability to provide similar optical input functions to single cells and monitor their response dynamics allows cell-to-cell variation in network properties to be identified (FIG. 12E).

### Discussion for Examples 1-9

Considerable information about GPCR activated signaling networks exists from extensive biochemical analysis. There is however limited information about how signaling networks govern complex single cell behaviors. It has been difficult to address this question because of a lack of effective methods. The approach developed here and described above fills this gap combining a set of unique properties that are not present in existing methods. It has the ability to exercise tight spatiotemporal control over receptor activity in a selected region that is ˜0.5% of a single cell surface area. It can be used to steer complex cellular behavior in precisely defined directions. It is capable of monitoring the dynamics of an entire signaling network in a live cell without disruptive interventions during the execution of various stages of a cellular response. It provides precise control over the signaling input. The opsin based signaling triggers allow a cell to respond directly to an extracellular signal, which can be varied continuously and almost instantaneously in space, intensity, duration and time intervals. The optical approach here can essentially provide control over all GPCR activated signaling networks in any cell type. Reversibility allows tight temporal control and helps measure response dynamics accurately. Reproducibility allows the same input to be provided to different parts of the same cell or various cells to examine heterogeneity in responses. The ability to apply repeated inputs helps sustain cell responses that often occur over minutes or hours. Since they stimulate the endogenous pathway in a cell, the native pathway integrity is wholly maintained and the evoked cell response accurately reflects normal cell behavior. The ability to image responses to localized optical activation without globally activating the opsin helps quantitatively monitor cellular and molecular response dynamics.

This approach was applied to create a series of inputs varying in time and space continually to single neurons. Input patterning elicited spatially selective neurite initiation and coordinated control over extension-retraction cycles were elicited. The primary aim of these experiments was to validate the optical approach. However, the ability to optically elicit complex behavioral responses from a single neuron suggests that specific configurations of GPCR signals in space and time can govern early neuron differentiation in vivo. They also suggest that patterns of neurite growth during differentiation are determined by rapidly acting feedback mechanisms, which curtail distal growth when a neurite encounters an extracellular signal. This finding is consistent with a previously untested prediction that the neurite extension-retraction cycles are mediated by negative feedback. Finally, optical control over neurite growth may be of value in regenerating neuronal connections and for generating neuronal networks.

The ability to control single cell behavior using discretely directed optical inputs overcomes limits of traditional approaches where population analysis of ensemble effects can mask network properties of single cells. This approach was therefore used to interrogate signaling network dynamics, quantify network parameters, and identify systems level control at the basis of single cell migration. PIP3 dynamics was followed in a single immune cell that was optically orchestrated continually through migration initiation, sustained directionally sensitive migration, and adaptation. This allowed the identification of network properties that govern these critical events that have not been detected before. When initiating and maintaining migration, an ultrasensitive PIP3 response was detected at the cell front. Simultaneously, rapid depletion of PIP3 below basal level was observed at the back.

Ultrasensitivity is characterized by a sigmoidal response which, after an initial lag allows a large output to a relatively small input. It has been found that the switch like output in an ultrasensitive response helps a cell make a dedicated all or none decision in oocyte maturation in response to changing environmental conditions. The ultrasensitive PIP3 response detected here is thus consistent with the decisive nature of migration initiation, which shifts the cell from one state to another. Cell migration is initiated at the switch-like region with low variability among individual cells. A typical Michaelis-Menten response (n_{H}=1) requires an 81-fold change in the input to bring about an output response from 10% to 90% activation. However, only a 2-4-fold change in input was required for PIP3 production at the front to reach the switch like state when migration is initiated. Additionally, the threshold for migration initiation in the ultrasensitive response ensures that cells filter out noise due to random fluctuations in naturally occurring stimuli. The PIP3 ultrasensitivity at the front and decay at the back, provide a parsimonious mechanism that while amplifying the gradient ensures that noise is filtered. The rapid reversal of PIP3 gradient on reversal of migration and the collapse of the PIP3 gradient when a cell adapts to a stationary signal further supports a role for this gradient in directionally sensitive migration.

Single cell analysis so far has concentrated on transcripts, genes, secreted molecules and metabolites. There is little information on the cell behavior dynamics and network parameters unique to a single cell. It is challenging to quantify the network properties in a single cell and classify the heterogeneous population on the basis of systems characteristics. By using the opsin-based triggers to examine single cells individually and continually, it was demonstrated in the Examples that there are distinct differences in the network properties and migratory behavior among individual cells. Heterogeneity in sensitivity across single cells may confer the advantage of responsiveness to a wide range in stimulus concentration. The optical approach here can be used more widely to examine if such heterogeneity is a widespread property of GPCR mediated signaling. GPCR networks are the most important targets for therapeutic drugs. Identifying molecular differences between cells in network properties may help evolve better strategies for personalized therapeutics.

Approaches to control the spatiotemporal dynamics of signaling activity in selected regions of a single cell have been limited. The methods described here that optically control signaling at subcellular resolution are a step towards overcoming this limitation. The ability of a color opsin from the retina to recruit an entire signaling network native to hippocampal neurons or immune cells and orchestrate intricate patterns of cell behavior suggests that GPCR activated networks mediating complex single cell events are essentially ‘hard-wired’. The optical methods described here can thus be used to probe the network level control of a variety of additional GPCR initiated cell behaviors such as cardiomyocyte contraction, hormone secretion and neuron function. The reengineered CrBlue construct shows that it is possible to expand the repertoire of optical triggers by creating novel combinations of spectral tuning and G protein specificity. The approach described here can also be developed to optically instruct cell behavior such as morphogenetic migration and neuron differentiation in a whole animal.

### Experimental Procedures for Examples 1-9

### Constructs

All constructs were made in pcDNA3.1 from Invitrogen. All DNA analysis was done using NCBI and alignment of opsins was done using ClustalW software. bOpsin mCherry was created by subcloning bOpsin into the EcoRI-NotI and mCherry into the NotI-XbaI sites of pcDNA3.1 (Invitrogen). A synthetic chimera (CrBlue) was created (Integrated DNA Technologies) and subcloned into the EcoRI-NotI sites of pcDNA3.1. The CrBlue construct is the bOpsin containing the intracellular loops and most of the C terminus of the jellyfish opsin (FIG. 6D). CrBlue also contained the last 8 amino acids of rhodopsin (ETSQVAPA). mCherry (from R.Tsien) was fused to the C terminus of CrBlue (NotI-XbaI) to make the CrBlue mCherry in pcDNA3.1. DenMark, the dendritic marker from B.A. Hassan was excised from pUAST using Pmel and XbaI and subcloned into the EcoRV-XbaI sites of pcDNA3.1. mCherry Gγ9 was made by subcloning mCherry into the HindIII-KpnI sites and γ9 into the KpnI-EcoRI sites of pcDNA3.1. Plasmids were transformed into Top10 cells (Invitrogen), using Ampicillin as a selection marker, selected by PCR screening and confirmed by sequencing. YFP-γ9 as previously described.

Blue, green and red opsins were provided by D.Oprian (University of Michigan), Melanopsin by I. Provencio (University of Virginia, VA), Jellyfish opsin by Terakita (Osaka City University, Osaka), Rhodopsin by S. Karnik (Cleveland Clinic, OH). PH domain was obtained from T. Balla (National Institutes of Health, Bethesda, Md.), mGFP-Actin from Ryohei Yasuda (Addgene No. 21948), Akt-PH-GFP from Craig Montell (Addgene No. 18836), EGFP-Rac1 from Gary Bokoch (Addgene No. 12980), EGFP-Rac1-T17N from Gary Bokoch (Addgene No. 12982), GFPΔ-epac-mCh cAMP sensor from K. Jalink (Netherlands Cancer Institute, Netherlands).

### Cell Culture and Transfections

Cells were cultured using standard protocols. 1-2 day post natal hippocampal neuronal precursors were prepared on 0.15% agarose coated 29 mm glass bottom dishes. Cell suspensions were prepared from postnatal day 1-2 rat hippocampus using papain and mechanical dispersion and cultured as previously described. Neurons were transfected next day and optical activation experiments were conducted 24 hours later. HeLa cells (ATCC) were cultured in MEM containing 10% dialyzed fetal bovine serum (Atlanta Biologicals) and antibiotics. 0.1×10⁶ cells were seeded in 29 mm glass bottom dishes (In Vitro scientific) the day before transfections. Early passage RAW 264.7 cells (Tissue Culture Support Center at Washington University) were grown in DMEM with 10% dialyzed fetal bovine serum, antibiotics and L-glutamine (2 mM). They were seeded at a density of 0.1×10⁶ cells in 29 mm glass bottom dishes and transfected the same day. All cell types used here were transfected using Lipofectamine 2000 as per manufacturer's protocol. The amount of lipofectamine used to transfect a 29 mm dish containing 1-2×10⁵ cells: HeLa cells: 2 μl, Raw cells: 4 μl, Primary hippocampal neurons: 4 μl. Cells were incubated 4.5-5 hours with transfection medium (Opti-MEM Reduced Serum Medium, lipofectamine and ˜1 μg of each cDNA) and then replaced with regular medium. Cells were imaged after 16 hrs for optical activation.

### Measuring Space and Time Variant GPCR Activity

Global or spatially confined GPCR activity was measured using an assay developed by the inventors to study GPCR mediated Gβγ translocation. The time course of FP-γ9 intensity was monitored to measure GPCR activity.

The Gβγ9 translocation directly reflects the real time active status of GPCRs in living cells. This property was used to quantify GPCR activity in real time in a selected region of a cell or in a whole cell. FP tagged Gβγ9 is predominantly present on the plasma membrane when GPCRs are inactive. On GPCR activation βγ9 translocates to internal membranes, drastically decreasing the fluorescence on the plasma membrane and increasing the fluorescence in the Golgi and endoplasmic reticulum. In contrast to cytosolic secondary messengers, GPCRs and heterotrimeric G proteins possess slow plasma membrane diffusion rates. Here, these properties were employed to develop Gβγ9 translocation as a fast transient assay to detect localized GPCR activity in living cells. Quantification of GPCR activity included the following steps. First, a confocal image of the Gβγ9 distribution was captured. Second, ROIs (regions of interest) for localized optical activation of GPCRs were drawn on the initial image using Andor IQ. Third, a time lapse imaging protocol was created which usually contained two segments. (i) Basal time lapse imaging: To capture a series of images of the basal state of the cell before the onset of OA. Excitation wavelengths that do not globally activate the opsin were assigned. (ii) OA+time lapse imaging: To activate opsins in a restricted area of the cell or the entire cell depending the on the ROIs drawn. An appropriate wavelength around the λₘₐₓ wavelength of the opsin was allocated. Multiple OA and imaging segments were created if necessary by varying the pulse frequency and intervals between OA cycles depending on the experiment. Finally, the protocol was executed. The resultant time-lapse image series was analyzed to calculate mean Gβγ9 fluorescence intensities in selected plasma membrane and internal membrane regions while subtracting background. Due to cell to cell variation, usually these intensities were normalized to their basal level.

### Imaging Setup

For imaging, cells were seeded in 29 mm glass bottom tissue culture dishes. HBSS (Hank's Buffered Salt Solution) supplemented with 1 g/L glucose was used as the imaging buffer in all experiments. All imaging was performed with a spinning disk confocal imaging system comprising a Leica DMI6000B inverted microscope, a Yokogawa CSU-X1 spinning disk unit, Andor FRAPPA (fluorescence recovery after photobleaching (FRAP) and photoactivation (PA) unit, laser combiner with 50 mW 445, 488, 515 and 594 nm solid state lasers and iXon+EMCCD camera. This system is capable of high speed 4D image acquisition, exposing a stationary or moving selected area to a light beam of desired intensity and wavelength for defined durations of time and live data acquisition. The environmental chamber on the microscope was at 37° C. and dishes were masked with a transparent CO₂ mask to maintain humidified 5% CO₂ over the cells. Adaptive corrective focus was used in order to prevent focus drift during time lapse imaging experiments. All imaging experiments were conducted using a 63×, 1.4HCX apochromat objective. In experiments involving opsin activation, dishes were kept completely in the dark from the time of addition of 11-cis retinal. Depending on the opsin, wavelengths other than its λ max were used to visualize cells.

### Time Lapse Imaging

Live imaging was performed using a Leica-Andor spinning disc confocal imaging system which comprises an Andor FRAPPA device, EM-CCD camera, IQ image analysis software and intensity optimized specific wavelength lasers. In order to avoid anomalies due to confocal plane changes, an adaptive focus control was employed. Prior to activation opsin expressing cells were maintained in the dark.

Cells were incubated with 11-cis retinal (3 ng/ml) in the regular medium for 30 min before OA. Global activation of opsins was achieved by exposing cells to the appropriate imaging beam coming through the spinning disk and FRAPPA unit. Settings used for imaging are present in Table 7 and FIG. 4D. Opsins showing global activation under these conditions were considered unsuitable for spatially restricted activation. The FRAPPA laser targeting controls 3 parameters: the laser intensity, dwell time (per pixel) and repetitions of how many times a region is activated before imaging. Optical input area (ROI-Region of Interest) for spatially restricted activation varied from 5-100 μm² (in a single cell) while restricted whole cell activation ROIs were similar to cell size. The time required for the optical input to complete scanning 1 μm² (22 pixels) area was ˜0.9 ms. Multi band dichroic filters and 10 ms switching was used for OA, simultaneous imaging and FRAPPA actions. Minimum intensity of an individual laser beam that induced first detectable Pγ9 translocation was used to spatially restrict opsin activation. It was also ensured that, at these intensities, there was no photobleaching of tagged fluorescent proteins. Optical activation specifics for restricted opsin activation are shown in Table 8 and FIG. 4A-C. Before and after neuronal imaging, Z stack images were obtained by capturing images at 0.2 μm intervals by using a Prior Piezo stage.

TABLE 7
Specifications for imaging of molecular responses
during spatially confined optical activation of opsins

| Fluorescent | Wavelength | Power | Exposure |
|---|---|---|---|
| protein | (nm) | Range (μW) | Time (ms) |
| mCh | 594 | 16.2-25.6 | 20-40 |
| YFP | 515 | 14.5-127.3 | 30-50 |
| GFP | 488 | 1.2-1.8 | 10-30 |

TABLE 8
Specifications for optical activation of opsins

| | | | Minimum | | |
|---|---|---|---|---|---|
| | | | number of | | Dwell |
| | Wavelength | Power | pulses | | time |
| Opsin | (nm) | (μW) | required | Repetitions | (μs) |
| bOpsin | 445 | 5 | Single pulse | 1 | 80 |
| Melanopsin | 448 | 27 | Single pulse | 1 | 80 |
| CrBlue | 445 | 12.5 | 5-10 pulses | 1 | 80 |

### Confined Optical Activation of Opsins in a Single Cell

A photoactivation (PA) unit containing a computer controlled dual galvanometer scan head was used to generate spatially restricted single or multi-region optical inputs for opsin activation. To simulate the lowest possible agonist concentration, the lowest intensity of the incident activation beam that initiates detectable Gy translocation was determined. Desired optical input functions were generated by programming laser pulses with appropriate dwell times per pixel, pixel areas exposed, time intervals and repetitions and used to activate opsins at selected regions of a cell.

FRET Imaging to Measure cAMP Production

Sequential time-lapse imaging of GFP and mCh using the imaging system described was used to determine the CrBlue induced cAMP generation in HeLa cells. Selected cells in the microscopic field were optically activated every 5 s (445 nm, 5 NW, ROI covering the entire cells) and all the cells were imaged for FRET changes (GR_{(488 excitation/565emission)}/GG_{(488excitation/515emission}, GFP-Δepac-mCh cAMP sensor).

HeLa cells on 23 mm glass bottom dishes were transfected with GFP-Δepac-mCh cAMP FRET sensor using the protocol described above. 24 hours after transfection, dishes were transferred to an incubator in a dark room and 11-cis retinal was added to the medium (3 ng/ml). After incubation with 11-cis retinal for 30 minutes, the medium was replaced with HBSS warmed to 37° C. cAMP binds to GFP-Δepac-mCh sensor resulting in FRET decrease. FRET was continually measured by exciting at 488 nm while measuring donor emission using 515 nm (GG) and acceptor emission using 595 nm (GR) filters. Out of several cells expressing the FRET sensor and bOpsin, cells were randomly chosen for OA. Separate ROIs were drawn around those cells for selective photoactivation. After imaging basal FRET every 1 s for 100 s, selected cells were optically activated using 445 nm (3%, 50 mW) beam using FRAP-PA device at 1 s intervals and FRET imaging was continued. FRET was calculated as GG/GR ratio. Cells expressing the constructs in the same field that are not optically activated were considered as the control cells. FRET sensor functionality was assessed by measuring FRET after acceptor photobleaching.

### Relationship Between Number of Light Pulses-Extent of Ry9 Translocation Response Curve in a Single Cell

The degree of GPCR activity was measured with systematic increase of optical input in a single cell by quantifying the extent of Gβγ9 translocation which is known to be the direct indicator of active receptor levels. We calculated the normalized increase in YFP-tagged γ9 fluorescence intensity at the internal membrane for different number of light pulses (n) used to activate the GPCRs by using the following equation:

$$Normalized γ9 fluorescence intensity = \frac{γ9 fluorescence level at the internal membrane at n = n}{Basal γ 9 fluorescence level at the internal membrane}$$

Due to the inherent heterogeneity in a cell population, different cells have different levels of γ9 expression. This resulted in heterogeneity in peak Gγ9 translocation upon exposing cells to 25 light pulses. γ9 fluorescence intensity values for all cells were again normalized with respect to maximum peak γ9 fluorescence intensity. Normalized increase in FP-tagged γ9 fluorescence intensity was plotted as a function of number of light pulses for 7 experiments and found the best fit with a hyperbolic function,

$$y = \frac{{V}_{m} x}{( x + {K}_{m} )}$$

using Origin 8.6 (Vₘ=0.93, Kₘ=10.3, R²=0.98).

### Quantification of Spatial Confinement of GPCR Activity in a Single Cell

GPCR activity across an optically activated area using a confined optical input was measured using the extent of Gγ9 translocation. The fast deactivation property of color opsins (<30 s) was employed.

The degree of spatial confinement in optically induced GPCR activity in a single cell was determined. An optical input of 2×2 μm² radius was applied, with a 445 nm pulse and measured the γ9 translocation extent at different points across the cell membrane (from x=−5 to 5 μm) from optical input (taken as x=0) applied onto a cell as shown in FIG. 2C. The normalized increase in FP-tagged γ9 fluorescence intensity was plotted as described above as a function of distances across the cell membrane and found the best fit with Gaussian distribution equation,

$$y = {y}_{0} + \frac{A {e}^{\frac{- 4 ln ( 2 ) {( x - {x}_{c} )}^{2}}{{w}^{2}}}}{w \sqrt{\frac{π}{4 ln ( 2 )}}}$$

using Origin Pro8.6 (γ0=0.19, xc=−0.079, A=1.56, w=FWHM=1.79, R²=0.80). This 2-D Gaussian distribution equation and spatial symmetry was used to simulate the variation of normalized GPCR activity in a single cell in 3-D space. Normalized GPCR activity falls below 5% of the maximum GPCR activity in an area having a diameter of d1=3.5 μm. Considering this as the lowest set point for receptor activity, the lower limit for spatial confinement in GPCR activity that can be achieved was further quantified using optical activation in a single cell. Fraction of cell surface in which GPCRs are activated,

$$\frac{surface area above 5 \% relative GPCR activity}{Total surface area of cell} = \frac{{π ( 0.5 d 1 )}^{2}}{2 π r h + π {r}^{2}} = \frac{1}{177} = 0.5 \%$$

Here it is assumed that the cell is dome shaped with a radius, r=15 μm and height, h=10 μm. Similarly, fraction of cell surface exposed the optical input above 5% of the maximum intensity (in an area of diameter, d2=4.78) was calculated

$$\frac{surface area above 5 \% relative ligth intensity}{Total surface area of cell} = \frac{{π ( 0.5 d 2 )}^{2}}{2 π r h + π {r}^{2}} = \frac{1}{98} = 1 \%$$

### Quantification of Neurite Extension in Response to Varying Optical Input

Dynamic curves were obtained for lamellipodia growth and actin formation by quantifying the corresponding FP fluorescence intensity at the growth regions at different time points.

The neurite tips of post natal 1-2 day old hippocampal neurons were optically activated to determine the optical patterning required for neurite outgrowth as well as the dynamics of actin formation and neurite growth (length). Dynamic curves for lamellipodia growth and actin formation were obtained by quantifying the corresponding FP fluorescence intensity at the growth regions at different time points. The trajectory of the optical input during neurite initiation and extension were quantified using the Tracker video analysis and modeling tool (Open Source Physics Project). To obtain the space and time varying optical input function, the distance traveled by the moving optical input along the neurite growth axis was plotted against time. Similarly, to obtain real-time dynamics of neurite outgrowth, the neurite length at each time point were similarly measured. Linear slopes of the optical and the neurite tip trajectories were calculated using Origin 8.6.

### Real Time Analysis of Single Cell Migratory Responses

Using the tracker analysis tool, dominant features on the cell were marked and tracked over the entire time-lapse image stack. Similarly, the boundaries of the optical input were tracked. These analyses resulted in XY movements as a function of time that allowed monitoring directional changes in detail (θ).

### Distribution Analysis

The cell population was grouped based on their K and Nstart and corresponding cumulative distribution was obtained. The distribution was fitted with a Hill function:

$${N}_{cum} = \frac{{x}_{0.5}^{n}}{( {x}_{0.5}^{n} + {B}^{n} )} \qquad ( n = standard deviation , B = mean ) .$$

The derivative of this function yielded the bimodal normalized distribution (FIG. 12H).

### Mathematical Modeling of PIP3 Response

Enzyme kinetics were used to model the PIP3 generation at the optically activated portion of the cell. The set of equations (SI) were solved numerically using ode23 program of MATLAB (The Mathworks Inc. USA). The dynamic PIP3 response (PIP3_{f}=output) was quantified as normalized fractional PIP3 accumulation

$$PIP {3}_{f} = \frac{PIP 3 accumulation at n = n}{PIP 3 accumulation at n = nmax}$$

(nmax=number of pulse at which the PIP3 reaches its peak response) as a function of number of light pulses (n=time varying input stimulus).

Described here is the method of performing the mathematical modeling of the reaction network module in a subcellular region (cell front, FIG. 12A) in a single cell during migration initiation. The network parameters for the internal molecules (PIP3) and physiological responses (migration initiation) for the front module of a polarization response were quantified and system properties were investigated. The optical approach allows confinement of the GPCR activity to a restricted region (FIG. 1F, G, H and FIGS. 3C and D) and thereby to decompose the network analysis at a spatial level. In this model, cell to cell heterogeneity was simulated by varying the kinetic and feedback parameters.

Model Description:
1) The model assumes that the receptor (GPCR) activation is proportional to the amount of stimulus.
2) Rate of PI3K recruitment to the membrane is assumed to follow the Michael is-Menten kinetics with the number of optically-activated receptors. The underlying mechanism is that light activated receptors activate the heterotrimeric G-protein and cause dissociation of the Gβγ heterodimer that recruits PI3K at the membrane.
3) Experimental evidence was previously found for PIP3 mediated positive feedback loop on PI3K activation in neutrophils. Since mechanistic details are lacking, the model assumes a function (1+nd*[PIP3]ⁿ) for the positive feedback where further active PI3K is available according to the extent of feedback (n).
4) The model specifically focuses on the interactions at spatially confined optically activated regions on the membrane (front module). The model assumes degradation of PIP3 at a constant rate as PTEN diffuses out rapidly. Additionally, there is experimental evidence that PTEN translocates to the back of the cell in the presence of a chemoattractant gradient.

### Quantification of the Network Properties for Cell Migration

For quantification of PIP3 and physiological responses during migration, three parameters were used, Hill coefficient (n_{H}), the half maximal PIP3 response (K) and amplification factor (A), number of light pulses required to initiate migration (Nₛₜₐᵣₜ) describing the system properties.

Hill Coefficient and Activation Threshold:

Since the PIP3 response to increasing stimuli generated from the model was sigmoidal, the experimental PIP3 data obtained from optical activation of immune cells was fitted to a three parameter Hill equation,

$$y = \frac{b {x}^{nH}}{{K}^{nH} + {x}^{nH}} .$$

where the y-axis is the fractional PIP3 response at the optically activated region of the cell and x-axis is the number of light pulses. n_{H}=Hill coefficient measuring the sensitivity of the system and K=half maximal PIP3 response measuring the activation threshold in a system. Since different cells had different values of basal and peak PIP3 sensor fluorescence (on the membrane) and their peak response was reached after varying numbers of light pulses, the PIP3 response was normalized as the fractional change in PIP3 fluorescence, and a similar quantity described in our mathematical model was obtained.

$$\frac{PIP 3 fluorescence level - basal PIP 3 fluorescence level}{Peak PIP 3 fluorescence level - basal PIP 3 fluorescence level}$$

Alternatively, when combining data from different experiments to plot a common curve for them (FIG. 6i), four parameter Hill equations were used

$$y = a + \frac{b {x}^{nH}}{{K}^{nH} + {x}^{nH}}$$

This eliminated the possibility of underestimating ultrasensitivity due to averaging curves. It is possible that there is some variability in terms of enzyme level or the PIP3 sensor level that leads to such variability in sensitivity.

Amplification (A):

A PIP3 amplification analysis was also performed, where amplification factor was calculated from experimental PIP3 response curves as:

$$A = \frac{Peak PIP 3 fluorescence level at the membrane}{Basal PIP 3 fluorescence level at the membrane}$$

In these plots, the PIP3 response is normalized to the maximum PIP3 response obtained in 23 cells (FIG. 12F, y-axis, left) and the input level is normalized to the total number of light pulses required for maximal PIP3 response and termed as fractional activation (FIG. 12F, x-axis).

Quantification of PIP3 Response in a Single Cell During Repeated Switching of Optical Input

The PIP3 response during the entire time of multiple switching cycles was segmented based on the switching location of the optical input. For each segment, different values were obtained for the ratio of peak to basal PIP3 sensor fluorescence. Therefore, the PIP3 response was normalized for each segment (from 0 to 1) as the fractional change in PIP3 fluorescence defined as,

$$\frac{\begin{matrix}PIP 3 fluorescence level at any time t - \\ basal PIP 3 fluorescence level\end{matrix}}{Peak PIP 3 fluorescence level - basal PIP 3 fluorescence level}$$

PIP3 Front/Back Gradient Analysis

A PIP3 gradient analysis was performed during migration and adaptation, where two amplification factors were calculated at each time point as follows,

$${A}_{front / back} = \frac{PIP 3 fluorescence level at the front membrane}{PIP 3 fluorescence level at the back membrane} and$$

$${A}_{front} = \frac{PIP 3 fluorescence level at the front membrane}{PIP 3 fluorescence level in cyotplasm} .$$

These amplification factors were plotted as a function of time to correlate the dynamics of PIP3 gradient with directional sensing and adaptation.

### Experimental Set Up Using Optical Activation of the Immune Cells.

In contrast to step-like signaling input used in traditional methods, a pulse-like input is more suitable to interrogate the signaling network in an unperturbed manner and understand the system characteristics. A series of light pulses (frequency, f=1 pulse/sec) were used to generate a controlled input that systematically increases the amount of stimulus and the signaling activity in a single cell (FIG. 1E). A very small portion of the cell front was optically activated (FIG. 1H, FWMH=1.79).

### Data Analysis Software and Statistics

All intensity recordings were background subtracted. Image analysis was performed using Andor IQ v2.4.1 and task specific Python scripts. Data analyses, curve fitting and statistical analysis associated with the corresponding functions were performed using Origin Pro 8.6 and Matlab (R2011 b). Cell and optical input co-ordinates were determined using Tracker video analysis and modeling tool. Detail statistical analyses are described in the methods section. Error bars represent Mean±standard error of the mean (SEM).

1. A method of modulating localized G protein signaling in a cell using an artificial optical input, the method comprising
(a) introducing at least one exogenous opsin into a cell, wherein (i) the exogenous opsin comprises a light sensing domain of a melanopsin or a metazoan color opsin and a G protein coupled receptor (GPCR) activation domain that effects G protein signaling (ii) and introducing exogenous opsin into a cell comprises introducing an amino acid sequence comprising an opsin into the cell, introducing a nucleic acid sequence capable of expressing an opsin into the cell, or a combination thereof; and
(b) changing an artificial optical input in a localized region on the cell's surface,
wherein the activation state of the exogenous opsin within the localized region is affected when the light sensing domain detects a change in the artificial optical input thereby resulting in the GPCR activation domain modulating G protein signaling.

2. The method of claim 1, wherein the exogenous opsin is selected from the group consisting of a melanopsin, a metazoan blue opsin, a metazoan green opsin, and a metazoan red opsin.

3. The method of claim 1, wherein the GPCR activation domain activates a G protein comprising a Gα subunit selected from the group consisting of a Gαs subunit, a Gαi/o subunit, a Gαq subunit, and Gα12/13.

4. The method of claim 1, wherein the light sensing domain is a metazoan color opsin and the metazoan color opsin is a mammalian blue opsin.

5. The method of claim 2, wherein the light sensing domain is a metazoan color opsin and the metazoan color opsin is a mammalian blue opsin.

6. The method of claim 1, wherein the localized region on the cell's surface is about 0.25% to about 50% of the cell surface area.

7. The method of claim 1, wherein the localized region on the cell's surface is input is no more than about 1% of the cell surface area.

8. The method of claim 1, wherein the cell is selected from the group consisting of an immune cell, a neuron, and a cardiac cell.

9. The method of claim 6, wherein the input is about 10-20% of the cell size and laser is about 5-10 μm away from the cell periphery.

10. The method of claim 6, wherein the input is about 20-40% of the cell size and the laser is about 10 μm away from the cell periphery.

11. A method of modulating cell behavior that is controlled by localized G protein signaling in the cell, the method comprising
(a) introducing at least one exogenous opsin into a cell, wherein (i) the exogenous opsin comprises an light sensing domain of a melanopsin or a metazoan color opsin and a G protein coupled receptor (GPCR) activation domain that effects G protein signaling, and (ii) introducing exogenous opsin into a cell comprises introducing an amino acid sequence comprising an opsin into the cell, introducing a nucleic acid sequence capable of expressing an opsin into the cell, or a combination thereof; and
(b) changing an artificial optical input in a localized region on the cell's surface,
wherein the activation state of the exogenous opsin within the localized region is affected when the light sensing domain detects a change in the artificial optical input thereby resulting in the GPCR activation domain modulating G protein signaling and cell behavior.

12. The method of claim 12, wherein the cell is an immune cell selected from the group consisting of a B cell, a T cell, a macrophage, a neutrophil, a dendritic cell and a monocyte, the cellular behavior is cell migration, and the GPCR activation domain activates a G protein comprising a Gαi/o subunit.

13. The method of claim 13, wherein the immune cell is a macrophage.

14. The method of claim 12, wherein the cell is a neuron, the cell behavior is neurite outgrowth, and the GPCR activation domain activates a G protein comprising a Gαi/o subunit.

15. The method of claim 12, the exogenous opsin is selected from the group consisting of a melanopsin, a metazoan blue opsin, a metazoan green opsin, and a metazoan red opsin.

16. The method of claim 15, wherein the extracellular light sensing domain is a metazoan color opsin and the metazoan color opsin is a mammalian blue opsin.

17. The method of claim 12, wherein the localized region on the cell's surface is about 0.25% to about 50% of the cell surface area.

18. The method of claim 12, wherein the localized region on the cell's surface is input is no more than about 1% of the cell surface area.

19. A method of modulating localized G protein signaling in at least one cell in a tissue using an artificial optical input, the method comprising
(a) introducing at least one exogenous opsin into a cell, wherein (i) the exogenous opsin comprises an light sensing domain of a melanopsin or a metazoan color opsin and a G protein coupled receptor (GPCR) activation domain that effects G protein signaling, and (ii) introducing exogenous opsin into a cell comprises introducing an amino acid sequence comprising an opsin into the cell, introducing a nucleic acid sequence capable of expressing an opsin into the cell, or a combination thereof; and
(b) changing an artificial optical input in a localized region on the cell's surface,
wherein the activation state of the exogenous opsin within the localized region is affected when the light sensing domain detects a change in the artificial optical input thereby resulting in the GPCR activation domain modulating G protein signaling in at least one cell in the tissue.

20. The method of claim 19, wherein the cell is a cardiomyocyte and the tissue is cardiac tissue.


SEQUENCE LISTING (6 sequences)

SEQ ID NO: 1 - protein, 355 aa, Homo sapiens
MRKMSEEEFYLFKNISSVGPWDGPQYHIAPVWAFYLQAAFMGTVFLIGFPLNAMVLVATLRYKKLRQPLNYILVNVSFGGFLLCIFSVFPVFVASCNGYFVFGRHVCALEGFLGTVAGLVTGWSLAFLAFERYIVICKPFGNFRFSSKHALTVVLATWTIGIGVSIPPFFGWSRFIPEGLQCSCGPDWYTVGTKYRSESYTWFLFIFCFIVPLSLICFSYTQLLRALKAVAAQQQESATTQKAEREVSMVVVMVGSFCVCYVPYAAFAMYMVNNRNHGLDLRLVTIPSFFSKSASIYNPIIYCFMNKQFQACIMKMVCGKAMTDESDTCSSQKTEVSTVSSTQVGPNETSQVAPA

SEQ ID NO: 2 - protein, 330 aa, Carybdea rastonii
MGANITELISGFLACVVFLSISLNMIVILTFYRLRHKLAFKDALMASMAFSDVVQAIVGYPLEVFTVVDGKWTFGMELCQVAGFFITALGQVSIAHLTALALDRYFTVCRPFVATAIHGSMRNAGMVIFVCWFYASFWAVLPLVGWSNYDVEGDGMRCSINWADDSPKSYSYRVCLFVFIYLIPVLLMVATYVLVQGEMKNMRGRAAQLFGSESEAALKNIKAEKRHTRLVFVMILSFIVAWTPYTFVAMWVSFFTKQLGPIPLYVDTLAAMLAKSSAMFNPIIYCFLHKQFRRAVLRGVCGRIVGGNAIAPSSTAVEPGQTLASGTAES

SEQ ID NO: 3 - protein, 356 aa, Artificial Sequence
  SYNTHESIZED
MRKMSEEEFYLFKNISSVGPWDGPQYHIAPVWAFYLQAAFMGTVFLIGFPLNAMVLVAFYRLRHKLAFLNYILVNVSFGGFLLCIFSVFPVFVASCNGYFVFGRHVCALEGFLGTVAGLVTGWSLAFLAFERYIVICRPFVATAIHGSMRNALTVVLATWTIGIGVSIPPFFGWSRFIPEGLQCSCGPDWYTVGTKYRSESYTWFLFIFCFIVPLSLICFSYTLVQGEMKNMRGRAAQLFGSESEAALKNIKAEKRHTRMVVVMVGSFCVCYVPYAAFAMYMVNNRNHGLDLRLVTIPSFFSKSASIYNPIIYCFMNKQFQRAVLRGVCGRIVGGNAIAPSSTAVEPNETSQVAPA

SEQ ID NO: 4 - DNA, 1119 bp, Homo sapiens
atggcacagcagtggtctctccagaggttagccggcaggcatcctcaagactcatatgaggactccacccagtcatcgatcttcacctacaccaactccaactccacgcgtggccctttcgaaggccctaactaccacatcgctccgcggtgggtgtaccacctgacgtccgtgtggatgatcttcgtggtgacagcttccgtgttcaccaacggcttagtgctggctgctaccatgaagtttaaaaagttacgccaccctctgaactggattctggtcaacctggctgtggcagatctggctgaaaccgtgatcgcttccacgatatccatcgtgaaccaggtgagcggttacttcgtcctaggccaccctatgtgcgtgctggagggttacaccgtaagcttatgcggtatcacaggcctgtggtctctggctatcatcagctgggagcgctggctggtggtgtgcaagcctttcggtaacgtgcgcttcgacgctaaactggcgatcgtgggtatcgctttctcctggatctggtctgcagtgtggacggcgcctccaatattcggttggtctagatactggcctcacggccttaagacctcctgcggaccggacgtgttctccggttcctcctatcccggggtgcagtcctacatgatcgtgctgatggtgacctgctgcatcatccctttagctatcatcatgctgtgctacctccaggtgtggctagctatccgcgctgtggccaagcagcagaaggagtccgagagtactcaaaaggcagagaaggaagtgaccaggatggtggtggtgatgatcttcgcttactgcgtgtgctggggcccttacaccttcttcgcatgcttcgccgctgctaaccctgggtacgcattccaccctctgatggcggccttaccggcctacttcgctaaatccgctaccatctacaaccctgtgatctacgtattcatgaaccgccagttccggaactgtatactccagctcttcggtaagaaggtggacgacggatccgaattgagctccgcttccaagaccgaggtgtcctccgtctcgagcgtgtctcctgccgagactagtcaggtggctcctgcttaa

SEQ ID NO: 5 - DNA, 1121 bp, Homo sapiens
atggcacagcagtggtctctccagaggttagccggcaggcatcctcaagactcatatgaggactccacccagtcatcgatcttcacctacaccaactccaactccacgcgtggccctttcgaaggccctaactaccacatcgctccgcggtgggtgtaccacctgacgtccgtgtggatgatcttcgtggtgatcgcttccgtgttcaccaacggcttagtgctggctgctaccatgaagtttaaaaagttacgccaccctctgaactggattctggtcaacctggctgtggcagatctggctgaaaccgtgatcgcttccacgatatcctgtgtgaaccaggtgtacggttacttcgtcctaggccaccctatgtgcgtgctggagggttacaccgtaagcttatgcggtatcacaggcctgtggtctctggctatcatcagctgggagcgctggatggtggtgtgcaagcctttcggtaacgtgcgcttcgacgctaaactggcgatcgtgggtatcgctttctcctggatctgggctgcagtgtggacggcgcctccaatattcggttggtctagatactggcctcacggccttaagacctcctgcggaccggacgtgttctccggttcctcctatcccgggtggcagtcctacatgatcgtgctgatggtgacctgctgcatcacccctttaagcatcatcgtgctgtgctacctccaggtgtggctagctatccgcgctgtggccaagcagcagaaggagtccgagagtactgaaaaggcagagaaggaagtgaccaggatggtggtggtgatggtgctggctttctgcttctgctggggcccttacgcattcttcgcatgcttcgccgctgctaaccctgggtaccctttccaccctctgatggcggccttaccggccttcttcgctaaatccgctaccatctacaaccctgtgatctacgtattcatgaaccgccagttccggaactgtatactccagctcttcggtaagaaggtggacgacggatccgaattgagctccgcttccaagaccgaggtgtcctccgtctcgagcgtgtctcctgccgagactagtcaggtggctcctgcttaagc

SEQ ID NO: 6 - DNA, 1566 bp, Mus musculus
atggactctccttcaggaccaagagtcttgtcaagcttaactcaggatcccagcttcacaaccagtcctgccctgcaaggcatttggaacggcactcagaacgtctccgtaagagcccagcttctctctgttagccccacgacatctgcacatcaggctgctgcctgggtccccttccccacagtcgatgtcccagaccatgctcactataccctaggcacggtgatcctgctggtgggactcacagggatgctgggcaatctgacggtcatctacaccttctgcaggaacagaggcctgcggacaccagcaaacatgttcatcatcaacctcgcagtcagcgacttcctcatgtcagtcactcaggccccggtcttctttgccagcagcctctacaagaagtggctctttggggagacaggttgcgagttctatgccttctgcggggctgtctttggcatcacttccatgatcaccctgacagccatagccatggaccgctatctggtgatcacacgtccactggccaccatcggcaggggatccaaaagacgaacggcactcgtcctgctaggcgtctggctttatgccctggcctggagtctgccacctttctttggttggagtgcctacgtgcccgaggggctgctgacatcctgctcctgggactacatgaccttcacaccccaggtgcgtgcctacaccatgctgctcttctgctttgtcttcttcctccccctgctcatcatcatcttctgctacatcttcatcttcagggccatccgagagacaggccgggcctgtgagggctgcggtgagtcccctctgcggcagaggcggcagtggcagcggctgcagagtgagtggaagatggccaaggtcgcactgattgtcattcttctcttcgtgctgtcctgggctccctactccactgtggctctggtggcctttgctggatactcgcacatcctgacgccctacatgagctcggtgccagccgtcatcgccaaggcttctgccatccacaatcccattatctacgccatcactcaccccaagtacagggtggccattgcccagcacctgccttgccttggggtgcttctcggtgtatcaggccagcgcagccacccctccctcagctaccgctctacccaccgctccacattgagcagccagtcctcagacctcagctggatctctggacggaagcgtcaagagtccctgggttctgagagtgaagtgggctggacagacacagaaacaaccgctgcatggggagctgcccagcaagcaagtggacagtccttctgcagtcagaacctagaagatggagaactcaaggcctcttccagcccccaggtacagagatctaagactcccaaggtgcctggacccagtacctgccgccctatgaaaggacagggagccaggccaagtagcctaaggggtgaccagaaaggcaggcttgctgtgtgcacaggcctctcagagtgtccccatccccatacatcccagtttccccttgctttcctagaggatgatgtgactctcagacatctgtag